(19) United States
(12) Patent Application Publication
(10) Pub. No.: US 2023/0181642 A1
(43) Pub. Date: Jun. 15, 2023

(54) IMMUNE CELLS WITH ENHANCED FUNCTION

(71) Applicant: The Council of the Queensland Institute of Medical Research, Herston, Queensland (AU)

(72) Inventors: Corey SMITH, Brisbane, Queensland (AU); Rajiv KHANNA, Brisbane, Queensland (AU); Katie LINEBURG, Brisbane, Queensland (AU); Archana PANIKKAR, Brisbane, Queensland (AU)

(21) Appl. No.: 17/926,106

(22) PCT Filed: Apr. 19, 2021
(86) PCT No.: PCT/AU2021/050347

§ 371 Date: Nov. 17, 2022

(30) Foreign Application Priority Data
Apr. 17, 2020 (AU) 2020901217

(51) Int. Cl. A61K 35/17; C12N 5/0783; C07K 14/725; A61P 37/00

(52) U.S. Cl. CPC A61K 35/17; C12N 5/0636; C07K 14/7051; A61P 37/00

(57) ABSTRACT

Disclosed are T-cells that are positive for CD49f and which have an enhanced function compared to CD49f− cells. Methods of isolation of CD49f+ T-cells, as well as compositions and kits thereof are also disclosed. Additionally, enriched CD49f+T-cell populations have an increased proliferative potential, long-term survival and significantly improved efficacy in an adoptive therapeutic setting. The CD49f+ T-cells and CD49f+ T-cell enriched T-cell populations are useful in a range of applications, including for use in treating or inhibiting the development of diseases with immune dysfunction and methods of assessing risk of disease and potential responsiveness in immunotherapy. CD 19 CAR-T cells derived from CD49f+ T-cells and their use in a method of treatment of cancer is also disclosed.

### RELATED APPLICATIONS

This application claims priority to Australian Provisional Application No. 2020901217 entitled “IMMUNE CELLS WITH ENHANCED FUNCTION” filed 17 Apr. 2020, the contents of which are incorporated herein by reference in their entirety.

### FIELD OF THE DISCLOSURE

Disclosed are immune cells with enhanced function. More particularly, the present disclosure relates to T-cells that are positive for CD49f and CD49f⁺ T-cell enriched T-cell populations with increased proliferative potential, long-term survival and significantly improved efficacy in an adoptive therapeutic setting. The CD49f⁺ T-cells and CD49f⁺ T-cell enriched T-cell populations are useful in a range of applications, including for use in treating or inhibiting the development of disease and in assessing risk of disease and potential responsiveness in immunotherapy

### BACKGROUND OF THE DISCLOSURE

Immunotherapy is a viable therapeutic methodology for infectious disease, chronic malignancies, and autoimmune disorders. Although immunotherapeutics come in many different forms, cellular immunotherapy is likely to be at the core of future disease treatment due in large part to its ability to direct antigen-specific immune effector cells to diseased cells, and to providing measurable clinical benefit in patients who are otherwise refractory to conventional therapy.

Cellular immunotherapies rely on the selection, expansion, and growth of effector leukocytes, the particulars of which, are highly variable. Numerous protocols and techniques are being evaluated for their ability to successfully manufacture large numbers of efficacious immune effector cells. However, there is currently no standard or validated method for evaluating a cell therapy product's potential in vivo efficacy.

Adoptive T-cell therapy (ACT) is a form of cellular immunotherapy, which involves administration of therapeutic T-cells to patients in order to treat disease, including cancer and viral infection (Rosenberg et al., Nat Rev Cancer, 2008. 8(4): 299-308; Gattinoni et al., Nat Rev Immunol, 2006. 6(5): 383-93; Fuji et al., Best Pract Res Clin Haematol. 2011. 24(3): 413-419; Khanna et al., Indian J Med Res. 2013. 138(5): 796-807).

Although both polyclonal and antigen-specific T-cells can be readily isolated from whole blood for ACT, their numbers are limited. Accordingly, protocols that activate and promote ex vivo expansion of T-cells are widely used for a wide variety of T-cell sources including genetically engineered, chimeric antigen receptor T-cells, autologous T-cells and allogeneic T-cells. To generate the large numbers of antigen-specific T-cells required for ACT, T-cells are conventionally stimulated with antigen over many weeks, often followed by T-cell selection and sub-cloning. Such ex vivo manipulations, however, are normally coupled with substantial T-cell differentiation and usually result in short-lived effects, including short-lived survival and a lack of persistence and lack of in vivo expansion of the transferred T-cells. Thus, existing T-cell manufacturing processes produce an inferior T-cell product that is prone to exhaustion and loss of effector immune cell function.

### SUMMARY OF THE DISCLOSURE

The present disclosure is predicated in part on the determination that expression in T-cells of the stem cell biomarker, integrin protein α6 (also known as CD49f), correlates with expression of key transcriptional regulators, T-cell factor 1 (TCF-1) and/or lymphoid enhancer binding factor 1 (LEF-1), which are associated with maintaining T-cell sternness and responsiveness in immunotherapy. Notably, CD49f⁺ T-cells are shown herein to have increased proliferative potential and retention of early memory and/or stem-like characteristics and long-term survival, with significantly improved efficacy in an adoptive therapeutic setting. These findings have been reduced to practice inter alia in isolated T-cell populations that are suitable for use in adoptive T-cell therapies in methods for assessing competence of a T-cell population for immunotherapy, in methods for enhancing immune effector function in a patient, and in pharmaceutical compositions, articles of manufacture and kits for use in those applications, as described hereafter.

Accordingly, disclosed herein in one aspect is an isolated T-cell population that comprises CD49f⁺ T-cells wherein the CD49f⁺ T-cells constitute at least 1% (including at least 2% to 99% and all integer percentages therebetween) of the T-cells in the population. In accordance with the present disclosure, the CD49f⁺ T-cells have enhanced immune properties, representative examples of which include one or more of an early memory phenotype, a stem-like phenotype, increased proliferative potential, increased survival and increased persistence in vivo, decreased differentiation, increased immune effector function, decreased immune effector dysfunction and increased responsiveness in immunotherapy. In some embodiments, the CD49f⁺ T-cells comprise CD49f^{hi} T-cells, CD49f^{int} T-cells, or both. In some of the same and other embodiments, the CD49f⁺ T-cells comprise memory T-cells (e.g., central memory T-cells) such as, but not limited to, CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T- cells, CD49f⁺ CD27⁺ CD28⁺ CD95⁺ memory T-cells, CD49f⁺ CD27⁺ CD28⁺ CD45RA⁺ CD95⁺ memory T- cells, CD49f⁺ CD27⁺ CD28⁺ CD95⁺ CCR7⁺ memory T-cells and CD49f⁺ CD27⁺ CD28⁺ CD45RA⁺ CD95⁺ CCR7⁺ memory T-cells. In illustrative examples of this type, the memory T-cells are positive for CD127. In some of the same and other embodiments, the CD49f⁺ T-cells are positive for one or both of CD4 and CD8. In some of the same and other embodiments, the CD49f⁺ T-cells have an early memory phenotype and/or a stem-like phenotype, which are also referred to herein as “young” or “potent” T-cells. In illustrative examples of this type, the CD49f⁺ T-cells are positive for TCF-1 (e.g., TCF-1^{hi}) and/or LEF-1 (e.g., LEF-1^{hi}) and optionally positive for one or both of Oct4 and Sox2. In some of the same and other embodiments, the CD49f⁺ T-cells in the isolated population constitute 1% or more of the T-cells in the population, including 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or up to and including 100% of the T-cells in the isolated population. In some of the same and other embodiments, the CD49f⁺ T-cells in the isolated population constitute 1% or more of the total number of cells in the population, including 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or up to and including 100% of the total number of cells in the isolated population. In specific embodiments, the isolated population is a substantially homogeneous population. In some embodiments, the CD49f⁺ T-cells express a recombinant T-cell receptor (rTCR). In some embodiments, the CD49f⁺ T-cells express a chimeric antigen receptor (CAR) and in non-limiting examples of this type, the CAR or CAR-expressing T-cell is suitably selected from a T-cell Redirected for Universal Cytokine Killing (“TRUCK”), Universal CAR, Self-driving CAR, Armored CAR, Self-destruct CAR, Conditional CAR, Marked CAR, TenCAR, Dual CAR, or safety CAR.

Disclosed herein in another aspect is a process of manufacturing a T-cell population comprising T-cells with enhanced immune properties (e.g., selected from one or more of an early memory phenotype, a stem-like phenotype, increased proliferative potential, increased survival and increased persistence in vivo, decreased differentiation, increased immune effector function, decreased immune effector dysfunction and increased responsiveness in immunotherapy), the process comprising or consisting essentially of: isolating or selecting from a sample containing T-cells a T-cell population comprising CD49f⁺ T-cells, wherein the CD49f⁺ T-cells constitute at least 1% (including at least 2% to 99% and all integer percentages therebetween) of the T-cells in the population, or enriching a sample containing T-cells for CD49f⁺ T-cells, thereby manufacturing a T-cell population comprising T-cells with enhanced immune properties. In some embodiments, the process further comprises harvesting the T-cell-containing sample from a suitable source. The source may be a peripheral blood mononuclear cell (PBMC) sample, cord blood cells, a purified population of T-cells, a T-cell line, or a sample obtained by leukapheresis. The T-cell-containing sample can be enriched for T-cells of interest, for example CD8⁺ T-cells, CD4⁺ T-cells, memory T-cells, previously activated T-cells and/or tumor infiltrating lymphocytes. Representative examples of CD49f⁺ T-cells include CD49f⁺ memory T-cells including CD49f⁺ central memory T-cells (e.g., CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T- cells, CD49f⁺ CD27⁺ CD28⁺ CD95⁺ memory T-cells, CD49f⁺ CD27⁺ CD28⁺ CD45RA⁺ CD95⁺ memory T- cells, CD49f⁺ CD27⁺ CD28⁺ CD95⁺ CCR7⁺ memory T-cells or CD49f⁺ CD27⁺ CD28⁺ CD45RA⁺ CD95⁺ CCR7⁺ memory T-cells, wherein any one of these memory T-cells is optionally CD8⁺, CD4⁺ or CD8⁺ CD4⁺). In illustrative examples of this type, the memory T-cells are positive for CD127. In some of the same and other embodiments, the CD49f⁺ T-cells have an early memory phenotype and/or a stem-like phenotype (e.g., CD49f⁺ T-cells are positive for TCF-1 (e.g., TCF-1^{hi}) and/or LEF-1 (e.g., LEF-1^{hi}) and optionally positive for one or both of Oct4 and Sox2). Suitably, the enhanced immune properties are relative to a control (e.g., a T-cell population that is not enriched for CD49f⁺ T-cells as defined above and elsewhere herein, or an isolated or CD49f⁺ T-cell enriched T-cell population as defined above and elsewhere herein). The isolated or CD49f⁺ T-cell enriched T-cell population has utility in immunotherapy, including in adoptive applications for treating or inhibiting the development of disease in a subject, and in representative embodiments of these applications, the isolated or CD49f⁺ T-cell enriched T-cell population may be autologous, allogeneic, or xenogeneic relative to the subject to whom the population is administered. In some embodiments, the isolation or enriching steps comprise contacting the sample T-cell population with an antigen-binding molecule that binds to CD49f and isolating cells that bind to the antigen-binding molecule. The anti-CD49f antigen-binding molecule may be directly or indirectly connected to a magnetic or paramagnetic particle and in non-limiting examples of this type, the enriching step comprises positive selection for CD49f⁺ cells using affinity based selection. In some of the same and other embodiments, the process further comprises isolating the T-cell-containing sample from a suitable source of T-cells, as for example described above and elsewhere herein. In some of the same and other embodiments, the process further comprises activating the T-cells of the isolated or CD49f⁺ T-cell enriched T-cell population. In some of the same and other embodiments, the process further comprises stimulating the T-cells of the isolated or CD49f⁺ T-cell enriched T-cell population to proliferate. In some non-limiting examples of this type, the activation and stimulation of the T-cells comprise contacting the T-cells with (1) an anti-CD3 antigen-binding molecule and (2) an anti-CD28 antigen-binding molecule, or B7-1 or B7-2. In some of the same and other non-limiting examples, the activation and stimulation of the T-cells comprise contacting the T-cells with an anti-CD49f antigen-binding molecule. In some of the same and other non-limiting examples, the process comprises contacting the T-cells with an antigen to produce antigen-specific T-cells. In some of the same and other embodiments, the process further comprises transducing the T-cells of the isolated or CD49f⁺ T-cell enriched T-cell population with a nucleic acid (e.g., a vector such as a viral vector including a retroviral vector such as a lentiviral vector) from which a rTCR or CAR is expressible, optionally in combination with a cytokine (e.g., an immune-stimulatory cytokine). Suitably, the T-cells are transduced with the nucleic acid after T-cell proliferation. In embodiments in which the nucleic acid expresses a CAR, the CAR suitably comprises a) an extracellular domain that binds to an antigen or portion thereof, wherein the antigen is selected from the group consisting of: a cancer or tumor-associated antigen, an infectious disease-associated antigen, an autoimmune disease-associated antigen, a transplantation antigen and an allergen; b) a transmembrane domain derived from a polypeptide selected from the group consisting of: CD8a, CD4, CD28, CD45, PD-1, and CD152; c) one or more intracellular costimulatory signaling domains selected from the group consisting of: CD28, CD54 (ICAM), CD134 (OX40), CD137 (413B), CD152 (CTLA4), CD273 (PD-L2), CD274 (PD-Li), and CD278 (ICOS); and d) a CD3-ζ signaling domain. Suitably, the extracellular domain comprises an antigen-binding molecule (e.g., scFv) that binds the antigen. The CAR may further comprise a hinge region polypeptide (e.g., a hinge region of IgG1 or CD8α). In some embodiments, the CAR further comprises a signal peptide (e.g., an IgG1 heavy chain signal polypeptide or a CD8a signal polypeptide). In some embodiments, the CD49f⁺ T-cells comprise a chimeric antigen receptor (CAR) and in non-limiting examples of this type, the process comprises transducing the T-cells of the isolated or CD49f⁺ T-cell enriched T-cell population with a nucleic acid (e.g., a vector such as a viral vector including a retroviral vector such as a lentiviral vector) from which a cytokine (e.g., an immune-stimulatory cytokine) is expressible. In some of the same and other embodiments, the process further comprises storing the isolated or CD49f⁺ T-cell enriched T-cell population. In representative examples of this type, the storing comprises cryopreservation of the isolated or CD49f⁺ T-cell enriched T-cell population.

Also disclosed herein is a kit for carrying out the manufacturing processes broadly described above and elsewhere herein, wherein the kit comprises antigen-binding molecules or other binding partners, generally coupled to solid supports, for the isolation or separation of, or enrichment for, a CD49f⁺ T-cell enriched T-cell population as broadly described above and elsewhere herein. Suitably, the kit includes an antigen-binding molecule for one or more or all T-cell biomarkers selected from CD95, CD45RA, CCR7, CD28, CD27, CD62L, CD127, and one or both of CD8 and CD4. In some embodiments, the kit contains instructional material for carrying out the isolation or separation of, or enrichment for, the CD49f⁺ T-cell enriched T-cell population. In some embodiments, the kit comprises antigen-binding molecules for positive and negative selection, bound to magnetic beads. In one embodiment, the kit comprises instructions to carry out selection starting with a sample, such as a PBMC sample, by selecting based on expression of a first surface marker, recognized by one or more of the antigen-binding molecules provided with the kit, retaining both positive and negative fractions. In some aspects, the instructions further include instructions to carry out one or more additional selection steps, starting with the positive and/or negative fractions derived therefrom, for example, while maintaining the compositions in a contained environment and/or in the same separation vessel.

Disclosed herein in yet another aspect is a method of determining a likelihood that a T-cell population is competent for immunotherapy (e.g., adoptive cell therapy), the method comprising or consisting essentially of: determining a level or concentration of CD49f⁺ T-cells in a sample of the T-cell population; and determining a likelihood that the T-cell population is competent for immunotherapy based on the level or concentration of CD49f⁺ T-cells in the sample. In some embodiments, the level or concentration of CD49f⁺ T-cells comprises a level or concentration of CD49f^{hi} T-cells only, a level or concentration of CD49f^{int} T-cells only, or a level or concentration of both CD49f^{hi} T-cells and CD49f^{int} T-cells. In some embodiments, the CD49f⁺ T-cells comprise memory T-cells (e.g., central memory T-cells), such as, but not limited to, CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T- cells, CD49f⁺ CD27⁺ CD28⁺ CD95⁺ memory T-cells, CD49f⁺ CD27⁺ CD28⁺ CD45RA⁺ CD95⁺ memory T- cells, CD49f⁺ CD27⁺ CD28⁺ CD95⁺ CCR7⁺ memory T-cells and CD49f⁺ CD27⁺ CD28⁺ CD45RA⁺ CD95⁺ CCR7⁺ memory T-cells. In illustrative examples of this type, the memory T-cells are positive for CD127.

In some of the same and other embodiments, the CD49f⁺ T-cells are positive for one or both of CD4 and CD8. In some of the same and other embodiments, the CD49f⁺ T-cells have an early memory phenotype and/or a stem-like phenotype. In illustrative examples of this type, the CD49f⁺ T-cells are positive for TCF-1 (e.g., TCF-1^{hi}) and/or LEF-1 (e.g., LEF-1^{hi}) and optionally positive for one or both of Oct4 and Sox2. In some of the same and other embodiments, the T-cell population is determined to be competent for immunotherapy when the level or concentration of CD49f⁺ T-cells meets or exceeds a threshold level or concentration that correlates with competence for immunotherapy. In illustrative examples of this type, the T-cell population is determined to be competent for immunotherapy when the level or concentration of CD49f⁺ T-cells is at least 1% of the T-cells in the population (including at least 2% and up to and including 100%, and all integer percentages between 2% and 100%) of the T-cells in the population. In other illustrative examples, the T-cell population is determined to be competent for immunotherapy when the level or concentration of CD49f⁺ T-cells is 1% or more of the total number of cells in the population, including 2% or more and up to and including 100% (and all integer percentages between 2% and 100%), of the total number of cells in the T-cell population. In other embodiments, the T-cell population is determined to be incompetent for immunotherapy when the level or concentration of CD49f⁺ T-cells is below a threshold level or concentration that correlates with competence for immunotherapy. In non-limiting examples of this type, the T-cell population is determined to be incompetent for immunotherapy when the level or concentration of CD49f⁺ T-cells is less than 1% of the T-cells in the population, including less than 0.9%, less than 0.8%, less than 0.7%, less than 0.6%, less than 0.5%, less than 0.4%, less than 0.3%, less than 0.2% or less than 0.1% of the T-cells in the population. In other non-limiting examples, the T-cell population is determined to be incompetent for immunotherapy when the level or concentration of CD49f⁺ T-cells is less than 1% of the total number of cells in the population, including less than 0.9%, less than 0.8%, less than 0.7%, less than 0.6%, less than 0.5%, less than 0.4%, less than 0.3%, less than 0.2% or less than 0.1% of the total number of cells in the population. Suitably, the T-cell population is an unexpanded population of T-cells. Alternatively, the T-cell population is an expanded population of T-cells. In some of the same and other embodiments, the T-cell population results from a process that includes antigen-specific stimulation of T-cells to produce antigen-specific T-cells.

Disclosed herein in a related aspect is a kit for determining a likelihood that a T-cell population is competent for immunotherapy (e.g., adoptive cell therapy), the kit comprising an antigen-binding molecule for detecting CD49f⁺ T-cells in the T-cell population. Suitably, the kit further includes an antigen-binding molecule for one or more (e.g., 1, 2, 3, 4, 5, 6, 7, 8, or 9) T-cell biomarkers selected from CD95, CD45RA, CCR7, CD28, CD27, CCR7, CD45RA, CD62L, CD127 and one or both of CD8 and CD4. In some embodiments, the kit contains instructional material for detecting and/or quantifying the CD49f⁺ T-cells in the T-cell population. The T-cell population may be a T-cell-containing sample or an isolated or CD49f⁺ T-cell enriched T-cell population as broadly described above and elsewhere herein.

Disclosed herein in still another aspect is a pharmaceutical composition comprising an isolated or CD49f⁺ T-cell enriched T-cell population as broadly described above and elsewhere herein, and optionally a pharmaceutically carrier.

Also disclosed herein in another aspect is an article of manufacture, comprising: one or more sealable containers individually comprising: at least one unit dose of an isolated or CD49f⁺ T-cell enriched T-cell population as broadly described above and elsewhere herein for administration to a subject; packaging material; and a label or package insert comprising instructions for administering the at least one unit dose to a subject by carrying out at least one administration. Suitably, the unit dose comprises about 1×10⁶ to about 5×10⁸ cells. In some embodiments, the article of manufacture comprises a plurality of unit doses and the label or package insert comprises instructions for administering the plurality of unit doses to the subject by carrying out a first administration and at least one subsequent administration, wherein the first administration comprises delivering one of the unit doses to the subject and the at least one subsequent administration individually comprises administering one or a plurality of said the doses to the subject. The isolated or CD49f⁺ T-cell enriched T-cell population may be autologous, allogeneic or xenogeneic relative to the subject to whom the population is administered.

Further disclosed herein in another aspect is a method for enhancing immune effector function in a patient having or at risk of developing an immune dysfunction, or requiring augmented immune effector function, the method comprising or consisting essentially of: administering to the patient an effective amount of an isolated or CD49f⁺ T-cell enriched T-cell population as broadly described above and elsewhere herein.

In a related aspect, a method is disclosed herein for treating or inhibiting the development of a condition in a patient, wherein the patient has or is at risk of developing an immune dysfunction and/or is in need or desirous of augmented immune effector function, the method comprising or consisting essentially of: administering to the patient an effective amount of an isolated or CD49f⁺ T-cell enriched T-cell population as broadly described above and elsewhere herein.

In some embodiments of these therapeutic aspects, the patient is in need of adoptive transfer of T-cells, suitably antigen-specific T-cells. In some of the same and other embodiments, the isolated or CD49f⁺ T-cell enriched T-cell population is autologous to the patient. In other embodiments, the isolated or CD49f⁺ T-cell enriched T-cell population is from a suitable donor who is suitably HLA-matched to the patient. In still other embodiments, the isolated or CD49f⁺ T-cell enriched T-cell population is from a xenogeneic source. In specific embodiments, the patient has or is at risk of developing a T-cell dysfunctional disorder. Suitably, the patient is a cancer patient, a patient having an infectious disease, a patient having autoimmune disease, or a patient in need of transplantation.

In another aspect, a method is disclosed herein for enhancing immune effector function in a patient having or at risk of developing an immune dysfunction, or requiring augmented immune effector function, the method comprising or consisting essentially of: contacting T-cells in the patient with an anti-CD49f affinity agent (e.g., an anti-CD49f antigen-binding molecule) to selectively stimulate activation of CD49f⁺ immune cells in the patient and enhance immune effector function in the patient.

In a related aspect, a method is disclosed herein for treating or inhibiting the development of a condition in a patient, wherein the patient has or is at risk of developing an immune dysfunction and/or is in need or desirous of an augmented immune effector function, the method comprising or consisting essentially of: contacting T-cells in the patient with an anti-CD49f affinity agent (e.g., an anti-CD49f antigen-binding molecule) to selectively stimulate activation of CD49f⁺ immune cells in the patient and treat or inhibit the development of the condition. Suitably, the condition is selected from cancer, infectious disease, autoimmune disease, inflammatory disease, and immunodeficiency.

In some embodiments of these therapeutic aspects, the anti-CD49f affinity agent (e.g., an anti-CD49f antigen-binding molecule) stimulates activation of CD49f⁺ T-cells, non-limiting examples of which include CD49f⁺ memory T-cells (e.g., CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T-cells, CD49f⁺ CD27⁺ CD28⁺ CD95⁺ memory T- cells, CD49f⁺ CD27⁺ CD28⁺ CD45RA⁺ CD95⁺ memory T-cells, CD49f⁺ CD27⁺ CD28⁺ CD95⁺ CCR7⁺ memory T-cells and CD49f⁺ CD27⁺ CD28⁺ CD45RA⁺ CD95⁺ CCR7⁺ memory T-cells). In illustrative examples of this type, the memory T-cells are positive for CD127. In specific embodiments, the patient has or is at risk of developing a T-cell dysfunctional disorder. Suitably, the patient is a cancer patient, a patient having an infectious disease, a patient having autoimmune disease, or a patient in need of transplantation. Suitably, the method comprises administering an effective amount of the anti-CD49f affinity agent (e.g., an anti-CD49f antigen-binding molecule) to the subject. In some of the same and other embodiments, the method further comprises concurrently administering with the anti-CD49f affinity agent (e.g., an anti-CD49f antigen-binding molecule) an ancillary agent that stimulates immune effector function or that treats or inhibits the development of the condition in the patient. In illustrative examples of this type, the ancillary agent comprises an immunotherapy such as an immune-checkpoint inhibitor.

### BRIEF DESCRIPTION OF THE DRAWINGS

FIG. 1 is a graphical representation showing that expression of CD49f defines distinct CMV-specific T-cell subsets. CMV-specific MHC-multimer binding CD8⁺ T-cells were sorted from CMV-seropositive donors (n=27) and assessed for gene expression using a customized gene expression array. (A) Representative MHC-multimer staining from two donors is shown. (B) Cluster analysis was performed using hierarchical clustering in R (C) Differential gene expression that define cluster 1 and 3. (D) PBMC from eight CMV-seropositive donors were assessed for the expression of CD49f in MHC-multimer⁺ populations by flow cytometry.

FIG. 2 is a graphical representation showing the association of CD49f expression with memory T-cell populations. PBMC were assessed for the expression of CD49f in distinct memory CD8⁺ T-cell populations defined by their expression of CD45RA, CCR7, CD27, CD8 and CD57. (A) Co-expression of CD49f with each phenotypic marker in naïve and central memory cells (B) Representative data of the gating strategy used to define memory populations and mean MFI of CD49f in these populations. (C) The proportion of CD49f^{hi}, CD49f^{int} and CD49f^{lo} T-cells in memory populations from two volunteers and two CMV-specific MHC-multimer⁺ CD8⁺ T-cells.

FIG. 3 is a graphical representation showing the association of CD49f expression and transcriptional regulation in CD8⁺ T-cells. PBMC were assessed for co-expression of CD49f in memory CD8⁺ T-cells with key transcriptional regulators, effector molecules and other T-cell related integrins. (A) Representative analysis of the expression of the transcriptional regulators T-bet, Hobit and Eomes, granzyme B and the integrin molecules CD29, CD11a and CD18 in CD49f^{hi}, CD49f^{int} and CD49f^{lo} memory CD8⁺ T-cells from a single donor. (B) Co-expression of self-renewal associated transcription factors, TCF-1 and LEF1 in CD49f^{hi}, CD49f^{int} and CD49f^{lo} memory CD8⁺ T-cells. Data represents the mean proportion of each cell population from three donors and representative data of the gating strategy used to define memory populations. (C) PBMC were labeled with cell trace violet, then sorted for CD49f^{hi}, CD49f^{int} and CD49f^{lo} memory CD8⁺. Sorted T-cells were stimulated with anti-CD3/anti-CD28 beads, then assessed for cell division after 4 days of culture.

FIG. 4 is a graphical representation depicting CMV-specific immune reconstitution following HSCT PBMC from a panel of R⁺ D− HSCT recipients at 1 month and 3 months post-transplant were assessed for expression of CD49f in CD8⁺ and CMV-specific MHC-multimer⁺ T-cells. (A) Representative flow cytometry analysis of CD49f expression in CD8⁺ T-cells at 1 month and 3 months post-transplant from two patients is showed. (B) Paired analysis of the proportion of CD49f^{lo} and CD49f^{hi} T-cells at 1 month and 3 months from 10 HSCT recipients. (C) Representative flow cytometry analysis of CD49f expression in CMV-specific MHC-Multimer⁺ T-cells at 1 month and 3 months post-transplant from two patients is showed. (D) Paired analysis of the proportion of CD49f^{lo} and CD49f^{hi} MHC-Multimer⁺ T-cells at 1 month and 3 months from 10 HSCT recipients. (E) Comparative analysis of the proportion of CD49f^{hi} and CD49f^{lo} CD8⁺ T-cells at 1 month and 3 months from HSCT recipients with either stable of unstable immunity. (F) Peak viral load in the first three months following HSCT in the peripheral blood of patients with stable or unstable immunity. (G) Longitudinal viral load (black line) overlaid with the proportion of CD49f^{lo} CD8⁺ T-cells (red bars) in two R⁺ D⁺ patient who developed CMV-associated diseases (dashed line).

FIG. 5 is a graphical representation showing impact of CD49f-expression on immune reconstitution post-ACT. (A) CMV-viral load in SOT patients treated with CMV-specific cells (B) Frequency of CMV-specific IFN-γ producing T-cells before and after ACT. (C) CMV-specific IFN-γ producing CD8⁺ T-cells in the cellular product. (D) CD49f expression in CD8⁺ T-cells prior to cell manufacture for ACT. (E) Correlation between the proportion of CD49flo CD8⁺ T-cells in starting PBMC and the expression of terminal differentiation (CD57) and memory markers (CD27. CD28) in T-cells generated for cell therapy.

FIG. 6 is a graphical representation depicting CD49f expressing T-cells retain increased proliferative potential after in vitro expansion. PBMC from a CMV-seropositive healthy volunteer were magnetically sorted into CD49f-positive and CD49f-lo populations, then stimulated with CMV-specific peptide pool designed for the generation of CMV-specific cellular therapy. Cells were cultured for 14 days in the presence of interleukin-2. (A) CD8⁺ T-cells from CD49f⁺ and CD49f^{lo} cultures were assessed for the co-expression of CD27 and CD28. (B) Cultured T-cells were labelled with cell trace violet then recalled with the CMV-specific peptide pool. Cells were assessed for proliferation by dilution of cell trace after 4 days.

FIG. 7 is a graphical representation showing that T-cells generated from the CD49f⁺ compartment show improved efficacy in a humanized model of Epstein Barr Virus associated lymphoma. PBMC were magnetically sorted into CD49f⁺ and CD49f− populations, then stimulated with EBV-encoded peptide epitopes pulsed onto autologous PBMC. T-cells were cultured in the presence of IL-2 for 17 days, assessed for EBV-reactivity then cryopreserved. Immunodeficient mice were injected subcutaneously with EBV-transformed B cells HLA matched to the CD49f⁺ and CD49f− T-cells. Mice were assessed for tumor formation, then after 16 days six mice per group were injected intravenously with 5 million T-cells generated from either the CD49f⁺ or CD49f− compartment. One day later mice were injected with anti-PD1 antibody. On day 20 and 21, mice were treated with a second dose or T-cells and anti-PD1 respectively. Mock mice received a mock injection of PBS and control IgG4. Mice were monitored for tumor growth until day 31.

FIG. 8 is a graphical representation showing association of LEF1, TCF1 and CD49f (ITGA6). Volcano plot of gene expression profiling from GSE140430. Genes in left cluster, including LEF1, TCF7 and ITGA6, are more highly expressed in stem-like tumour infiltrating T-cells.

FIG. 9 is a graphical representation showing differential gene expression in CD8+ T cells defined by CD49f expression levels. NanoString gene expression analysis of sort-purified CD8⁺ PBMC based on CD49f expression levels, into CD49f^{hi}, CD49f^{int} and CD49^{lo} populations. (A) Representative gating strategy used for isolation of T-cells based upon CD49f expression (B) A volcano plot displaying differential expression in 162 genes for samples sorted from healthy (n=7) individual donors. (C) Graphs represent the comparative gene expression identified between CD49f^{hi} (solid squares), CD49f^{int} (solid circles) and CD49^{lo} (open squares) populations. Significance was calculated using a Mann Whitney Test (* P<0.05), (** P<0.005) and (***P=0.0006).

FIG. 10 is a schematic representation showing efficacy of CAR19-T cells generated from the CD49f^{hi} compartment. (A) CD49f^{hi} and CD49f^{lo} memory T-cells were sorted using flow cytometry (FACSAriaIII) the stimulated with a-CD3 and a-CD28. After 48 hours, cells were transduced with a CAR-CD19 RFP lenitviral construct, then cultured for two weeks in the presence of IL-2. (B) Immunocompromised NOD-Rag1^{null} IL2rg^{null} mice were inject subcutaneously with 5×10⁵ BJAB cells (EBV⁻ Burkitt Lymphoma). Once tumours reached 25 mm² experimental groups were injected intravenously with two doses (96 hours apart) containing 2×10⁵ CAR19⁺ T-cells generated from the CD49f^{hi} or CD49f^{lo} compartment. Mice were monitored for tumour growth and sacrificed when tumour area reached a maximum of 150 mm². (C) At day 14, 21, 28 and 35 after T-cell infusion, mice were bled and assessed for the presence of human (CD45⁺) CAR19⁺ cells in the blood by RFP expression (n=5 mice/group).

### DETAILED DESCRIPTION OF THE DISCLOSURE

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “an element” means one element or more than one element.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “activation” refers to the state of a T-cell that has been sufficiently stimulated to induce detectable cellular proliferation. In particular embodiments, activation can also be associated with induced cytokine production, and detectable immune effector functions. The term “activated T-cells” refers to, among other things, T-cells that are proliferating. Signals generated through the TCR alone are insufficient for full activation of the T-cell and one or more secondary or co-stimulatory signals are also required. Thus, T-cell activation comprises a primary stimulation signal through the TCR/CD3 complex and one or more secondary costimulatory signals. Co-stimulation can be evidenced by proliferation and/or cytokine production by T-cells that have received a primary activation signal, such as stimulation through the CD3/TCR complex or through CD2.

The “amount” or “level” of a biomarker is a detectable level in a sample. These can be measured by methods known to one skilled in the art and also disclosed herein. The expression level or amount of biomarker assessed can be used to determine the response to treatment.

As used herein, “and/or” refers to and encompasses any and all possible combinations of one or more of the associated listed items, as well as the lack of combinations when interpreted in the alternative (or).

The term “anergy” refers to the state of unresponsiveness to antigen stimulation resulting from incomplete or insufficient signals delivered through the T-cell receptor (e.g. increase in intracellular Ca²⁺ in the absence of ras-activation). T-cell anergy can also result upon stimulation with antigen in the absence of co-stimulation, resulting in the cell becoming refractory to subsequent activation by the antigen even in the context of co-stimulation. The unresponsive state can often be overridden by the presence of IL-2. Anergic T-cells do not undergo clonal expansion and/or acquire effector functions.

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include polyclonal and monoclonal antibodies as well as their fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known. Antigen-binding molecules also encompass dimeric antibodies, as well as multivalent forms of antibodies. In some embodiments, the antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., 1984, Proc. Natl. Acad. Sci. USA 81:6851-6855). Also contemplated, are humanized antibodies, which are generally produced by transferring complementarity determining regions (CDRs) from heavy and light variable chains of a non-human (e.g., rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions. General techniques for cloning non-human, particularly murine, immunoglobulin variable domains are described, for example, by Orlandi et al. (1989, Proc. Natl. Acad. Sci. USA 86: 3833). Techniques for producing humanized monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies,” in Protein Engineering: Principles and Practice Cleland et al. (eds.), pages 399-434 (John Wiley & Sons, Inc. 1996), and by Queen et al., U.S. Pat. No. 5,693,762 (1997). Humanized antibodies include “primatized” antibodies in which the antigen-binding region of the antibody is derived from an antibody produced by immunizing macaque monkeys with the antigen of interest. Also contemplated as antigen-binding molecules are humanized antibodies.

The term “antigen-presenting cell” or “APC” refers to an immune system cell capable of displaying, acquiring, and/or presenting at least one antigen or antigenic fragment on (or at) its cell surface. In specific embodiments, an APC displays an endogenous or foreign antigen complexed with MHC on its surface. T-cells may recognize these complexes using their TCRs. APCs process antigens and present them to T-cells. APCs may be “loaded” with an antigen that is pulsed, or loaded, with antigenic peptide or recombinant peptide derived from one or more antigens. Specific non limiting examples of APCs include dendritic cells (DCs), dendritic cell-lines, B-cells, or B-cell-lines. The DCs or B-cells can be isolated or generated from the blood of a patient or suitable donor.

As used herein, the term “antigen-specific” refers to a property of a cell population such that supply of a particular antigen, or a fragment of the antigen, results in specific cell proliferation, suitably T-cell proliferation characterized for example by activation of the T-cells (e.g., CTLs and/or helper T-cells) that are suitably directed against a damaged cell, malignancy or infection.

As used herein, the term “antigen-specific T-cells” refers to T-cells that proliferate upon exposure to APCs or artificial antigen-presenting complexes (aAPCs), which present a cognate antigen in the context of MHC and suitably at least one T-cell co-stimulatory molecule (e.g., CD28, CD80 (B7-1), CD86 (B7-2), B7-H3, 4-1BBL, CD27, CD30, CD134 (OX-40L), B7h (B7RP-1), CD40, tumor necrosis factor superfamily member 14 (TNFSF14; also known as LIGHT), antibodies that specifically bind to herpesvirus entry mediator (HVEM), antibodies that specifically bind to CD40L, antibodies that specifically bind to OX40, and antibodies that specifically bind to 4-1BB). The term “antigen-specific T-cells” also refers to T-cells that are able to attack cells having the specific antigen on their surfaces. Such T-cells, e.g., CTLs, lyse target cells by a number of methods, e.g., releasing toxic enzymes such as granzymes and perforin onto the surface of the target cells or by effecting the entrance of these lytic enzymes into the target cell interior. Generally, CTLs express CD8 on their cell surface. T-cells that express the CD4 antigen, commonly known as “helper” T-cells, can also help promote specific cytotoxic activity and may also be activated by APCs or aAPCs. In certain embodiments, APCs and T-cells are derived from the same donor, which can be a patient or a suitable HLA-matched donor. Alternatively, the APCs and/or the T-cells can be allogeneic.

The term “autologous” refers to any material derived from the same individual to whom it is later to be re-introduced into the individual. The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically. The term “xenogeneic” refers to any material derived from an animal of a different species.

The terms “binds to”, “specifically binds to,” “specific for,” and related grammatical variants refer to that binding which occurs between such paired species as enzyme/substrate, receptor/agonist, antibody/antigen, nucleic acid/complement and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

As used herein, the term “biomarker” refers to a molecule that is associated either quantitatively or qualitatively with a biological activity or function (e.g., impaired or unimpaired or operable T-cell immune effector function). Examples of biomarkers include polynucleotides, such as a gene product, RNA or RNA fragment, polynucleotide copy number alterations (e.g., DNA copy numbers); proteins, polypeptides, and fragments of a polypeptide or protein; carbohydrates, and/or glycolipid-based molecular markers; polynucleotide or polypeptide modifications (e.g., posttranslational modifications, phosphorylation, DNA methylation, acetylation, and other chromatin modifications, glycosylation, etc.). In certain embodiments, a “biomarker” means a molecule/compound that is differentially present (i.e., increased or decreased) in a sample as measured/compared against the same marker in another sample or suitable control/reference. In other embodiments, a biomarker can be differentially present in a sample as measured/compared against the other markers in same or another sample or suitable control/reference. In further embodiments, one or more biomarkers can be differentially present in a sample as measured/compared against other markers in the same or another sample or suitable control/reference and against the same markers in another sample or suitable control/reference. In yet another embodiment, a biomarker can be differentially present in a sample from a subject or a group of subjects having a first phenotype (e.g., having a disease or condition) as compared to a sample from a subject or group of subjects having a second phenotype (e.g., not having the disease or condition or having a less severe version of the disease or condition).

The term “bispecific antigen-binding molecule” refers to an antigen-binding molecule having the capacity to bind to two distinct epitopes on the same antigen or on two different antigens. A bispecific antigen-binding molecule may be bivalent, trivalent, or tetravalent. As used herein, “valent”, “valence”, “valencies”, or other grammatical variations thereof, mean the number of antigen-binding sites in an antigen-binding molecule. These antigen recognition sites may recognize the same epitope or different epitopes. Bivalent and bispecific molecules are described in, e.g., Kostelny et al. J Immunol 148 (1992):1547, Pack and Pluckthun Biochemistry 31 (1992) 1579, Gruber et al. J Immunol (1994) 5368, Zhu et al. Protein Sci 6 (1997):781, Hu et al. Cancer Res. 56 (1996):3055, Adams et al. Cancer Res. 53 (1993):4026, and McCartney, et al. Protein Eng. 8 (1995):301. Trivalent bispecific antigen-binding molecules and tetravalent bispecific antigen-binding molecules are also known in the art. See, e.g., Kontermann R E (ed.), Springer Heidelberg Dordrecht London New York, pp. 199-216 (2011). A bispecific antigen-binding molecule may also have valencies higher than 4 and are also within the scope of the present invention. Such antigen-binding molecules may be generated by, for example, dock and lock conjugation method. (Chang, C.-H. et al. In: Bispecific Antibodies. Kontermann R E (2011), supra).

The term “cell population” refers generally to a grouping of cells. A cell population may consist of cells having a common phenotype (e.g., T-cells) or may comprise at least a fraction of cells having a common phenotype. Cells are said to have a common phenotype when they are substantially similar or identical in one or more demonstrable characteristics, including but not limited to morphological appearance, the presence, absence or level of expression of particular cellular components or products, e.g., RNA, proteins or other substances, activity of certain biochemical pathways, proliferation capacity and/or kinetics, differentiation potential and/or response to differentiation signals or behavior during in vitro cultivation (e.g., adherence, non-adherence, monolayer growth, proliferation kinetics, or the like). Such demonstrable characteristics may therefore define a cell population or a fraction thereof. Cell populations may be heterogeneous or homogeneous. When a cell population is said to be “heterogeneous”, this generally denotes a cell population comprising two or more cells or fractions of cells not having a common phenotype, e.g., a cell population comprising cells of two or more different cell types. By means of example and not limitation, a heterogeneous cell population can be isolated from blood, and may comprise peripheral blood mononuclear cells (PBMCs) which include lymphocytes (e.g., T-cells, B-cells, NK cells, etc.) and monocytes. When a cell population is said to be “homogeneous”, it consists of cells having a common phenotype. A cell population said herein to be “substantially homogeneous” comprises a substantial majority of cells having a common phenotype or biomarker signature. A “substantially homogeneous” cell population may comprise at least 70%, e.g., at least 80%, preferably at least 90%, e.g., at least 95%, or even at least 99% of cells having a common phenotype, such as the phenotype specifically referred to (e.g., a T-cell population having one or more of an early memory phenotype, a stem-like phenotype, increased proliferative potential, increased survival and increased persistence in vivo, decreased differentiation, increased immune effector function, decreased immune effector dysfunction and increased responsiveness in immunotherapy), or common biomarker panel (e.g., CD49f⁺ CD27⁺ CD28⁺, CD49f⁺ CD27⁺ CD28⁺ CD45RA⁺, CD49f⁺ CD27⁺ CD28⁺ CCR7⁺ and CD49f⁺ CD27⁺ CD28⁺ CD45RA⁺ CCR7⁺ and CD49f⁺ CD45RA⁺ CCR7⁺ CD28⁺ CD27⁺ TCF-1⁺LEF-1⁺). The term “T-cell population” refers to a cell population as defined herein comprising at least one T-cell and typically a fraction, more suitably a substantial fraction, of the population being T-cells. Usually, the T-cells of the fraction may have a common phenotype (e.g., CD8⁺, antigen-specificity, etc.). Examples of cell populations containing T-cells include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T-cells and derived from hematopoietic cells can be used in the present disclosure. These cells may have been activated by cytokine such as IL-2 in vivo or ex vivo. The term “T-cell population” is used interchangeably herein with “T-cell sample”.

As used herein, the term “cell surface marker” refers to proteins, carbohydrates, lipids, or combinations thereof, on the surface of the cells that can be used to discriminate a cell population.

The term “cognate antigen” refers to an antigen that is presented by a major histocompatibility complex (MHC) on an APC and to which a T-cell receptor (TCR), which has specificity for the antigen in the context of the MHC, binds thereby providing one of the signals for T-cell activation.

The term “competence for immunotherapy” as used in the present specification means the degree of competence of an immune cell-containing population for augmenting immune effector function. Immune cell-containing population samples may be classified in any way according to their competence for immunotherapy. For example, they may be divided into two classes, one which meets the standards for being competent and the other which falls short of those standards and being designated as incompetent. Alternatively, they may be divided into several classes which are ranked according to their competence for immunotherapy. In specific embodiments, immune cell-containing population samples are classified as being competent for immunotherapy when they have any one or more of the following immune effector characteristics: early memory phenotype, stem-like phenotype, increased proliferative potential, increased survival, increased immune effector function, decreased immune effector dysfunction and increased responsiveness in immunotherapy.

As used herein, a “composition” refers to any mixture of two or more products, substances, or compounds, including cells. It may be a solution, a suspension, liquid, powder, a paste, aqueous, non-aqueous or any combination thereof.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, a “co-stimulatory signal” refers to a signal, which in combination with a primary signal, such as TCR/CD3 ligation, leads to T-cell proliferation and immune effector functions such as, for example, cytokine production, cytolytic activity, and/or upregulation or downregulation of particular molecules (e.g., CD28). Thus, the term “co-stimulating”, “co-stimulation” and the like includes the ability of a co-stimulatory molecule to provide a second, non-activating receptor mediated signal (i.e., a “co-stimulatory signal”) that induces proliferation and immune effector function. As used herein the term “co-stimulatory molecule” includes molecules, which are present on (i) antigen presenting cells (e.g., B7-1, B7-2, B7RP-1, ICOSL, OX40L, 4-1BBL and/or related molecules that bind to co-stimulatory receptors (e.g., CD28, CTLA4, ICOS, OX40, 4-1BB and/or related molecules) on T-cells, and (ii) T-cells (e.g., CD40L, ICOS and/or related molecules that bind to co-stimulatory receptors (e.g., CD40, ICOSL and/or related molecules) on antigen presenting cells and B cells.

As used herein, “depleting” when referring to one or more particular cell type or cell population, refers to decreasing the number or percentage of the cell type or population, e.g., compared to the total number of cells in or volume of the composition, or relative to other cell types, such as by negative selection based on markers expressed by the population or cell, or by positive selection based on a marker not present on the cell population or cell to be depleted. The term does not require complete removal of the cell, cell type, or population from the composition.

As used herein, the term “differentiation” refers to a process of decreasing the potency or proliferation of a cell or moving the cell to a more developmentally restricted state. In particular embodiments, differentiated T-cells acquire immune effector functions.

The term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth.

The term “dysfunctional”, as used herein, also includes refractory or unresponsive to antigen recognition, specifically, impaired capacity to translate antigen recognition into down-stream T-cell effector functions, such as proliferation, cytokine production (e.g., IL-2, IFN-γ, TNF-α, etc.) and/or target cell killing.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the cancer or tumor. In the case of an infection, an effective amount of the drug may have the effect in reducing pathogen (bacterium, virus, etc.) titers in the circulation or tissue; reducing the number of pathogen infected cells; inhibiting (i.e., slow to some extent or desirably stop) pathogen infection of organs; inhibit (i.e., slow to some extent and desirably stop) pathogen growth; and/or relieving to some extent one or more of the symptoms associated with the infection. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer. A patient who “does not have an effective response” to treatment refers to a patient who does not have any one of extending survival (including overall survival and progression free survival); resulting in an objective response (including a complete response or a partial response); or improving signs or symptoms of cancer.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition. Representative enriching processes may result in a final cell population in which the percentage of one type of cell or subtype (e.g., CD49f⁺ T-cell or CD49f⁺ antigen-specific T-cell) is increased by about 0.01%, 0.05%, 0.1%, 0.2%, 0.3%, 0.4%, 0.5%, 1%, 2%, 5%, or 10%, by about 20%, by about 30%, by about 40%, by about 50% or by greater than 50% as compared to the percentage of the one type of cell in a starting or initial population of cells.

By the term “expanded population” is meant a population of cells, e.g., CD49f⁺ T-cells isolated from a T-cell source, e.g., peripheral blood, wherein at least 50% of the cells have divided at least once. Typically, the expanded population is enriched CD49f⁺ immune cells, suitably CD49f⁺ T-cells, relative to the population before expansion, by antigen stimulation.

As used herein, the term “expanding” when referring to cells, refers to increasing in cell number. In specific embodiments, the term “expanding” refers to promoting the growth or growing, particularly promoting the growth of a particular cell type (e.g., a CD49f⁺ immune cell such as a CD49f⁺ T-cell) within a mixed cell population. Expansion of T-cells is suitably performed by culturing a cell population comprising T-cells in the presence of T-cell- and/or antigen-specific T-cell-stimulating agent such as antigens, cells, including antigen-presenting cells, antibodies, lectins, etc. Expansion may also require culturing of T-cells in the presence of a cytokine.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The term “expression product” or “gene expression product” are used herein to refer to the RNA transcription products (transcripts) of a gene, including mRNA, and the polypeptide translation products of such RNA transcripts. An expression product can be, for example, an unspliced RNA, an mRNA, a splice variant mRNA, a microRNA, a fragmented RNA, a polypeptide, a post-translationally modified polypeptide, a splice variant polypeptide, etc.

The term “gene” as used herein refers to a DNA sequence which is expressed in a sample as an RNA transcript; a gene can be a full-length gene (protein encoding or non-encoding) or an expressed portion thereof, such as expressed sequence tag or “EST”. Thus, the genes described herein from which biomarkers of the disclosure are expressed (also referred to herein as “biomarker genes”) are each independently a full-length gene sequence, whose expression product is present in samples, or is a portion of an expressed sequence, e.g., EST sequence, that is detectable in samples. The biomarker genes and the sequences of those genes and biomarkers from which they are expressed, which are incorporated by reference herein, are found in the publicly available GenBank database by virtue of their gene identification numbers or Entrez Gene ID designations. Accordingly, all GenBank gene identification numbers and sequences related thereto are incorporated by reference in their entirety herein.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “HLA” means human leukocyte antigen and is equivalent to the term “major histocompatibility complex” (MHC) molecule. In general, class 1 molecules are MHC-encoded peptides that are associated with P2-microglobulin, while class 2 molecules have two non-covalently associated MHC encoded peptides. Class 1 (HLA-A, B, C) and 2 (HLA-D or DR, DQ, DP) molecules, when on the cell surface, are capable of presenting “antigens” that elicit an immune response. The term “HLA-matched donor” refers to an individual who expresses some or all of the seven different major histocompatibility complex (MHC) proteins on the cell surface in common with the intended recipient. In contrast, the term “allogeneic donor” indicates that the donor expresses none or few MHC proteins in common with the intended recipient. Whether or not two individuals are HLA-matched can be determined by standard tissue typing techniques using antibodies or by mixed lymphocyte reactions (MLR).

As used herein, the term “immune cell” refers to cells of the innate and acquired immune system including neutrophils, eosinophils, basophils, monocytes, macrophages, dendritic cells, lymphocytes including B cells, T-cells, and natural killer cells.

The term “immune effector cell” as used herein refers to any cell of the immune system that has one or more immune effector functions (e.g., cytotoxic cell killing activity, secretion of cytokines, induction of antibody-dependent cellular cytotoxity (ADCC) and/or cell-mediated cytotoxity (CDC)). Illustrative immune effector cells contemplated herein are T lymphocytes, in particular cytotoxic T-cells (CTLs; CD8⁺ T-cells), TILs, and helper T-cells (HTLs; CD4⁺ T-cells), as well as NK cells and NK T-cells.

The term “immune effector function” in the context of the present disclosure includes any function mediated by components of the immune system, particularly T-cells, which result, for example, in the killing of virally infected cells or tumor cells, or in the inhibition of tumor growth and/or inhibition of tumor development, including inhibition of tumor dissemination and metastasis. Preferably, the immune effector functions in the context of the present disclosure are T-cell mediated effector functions. Such functions comprise in the case of a helper T-cell (CD4⁺ T-cell) the recognition of an antigen or an antigen peptide derived from an antigen in the context of MHC class II molecules by T-cell receptors, the release of cytokines and/or the activation of CD8⁺ lymphocytes (CTLs) and/or B-cells, and in the case of CTL the recognition of an antigen or an antigen peptide derived from an antigen in the context of MHC class I molecules by T-cell receptors, the elimination of cells presented in the context of MHC class I molecules, i.e., cells characterized by presentation of an antigen with class I MHC, for example, via apoptosis or perforin-mediated cell lysis, production of cytokines such as IFN-γ and TNF-α, and specific cytolytic killing of antigen expressing target cells.

As used herein, the term “immune checkpoint inhibitor” or “checkpoint inhibitor” refers to molecules that totally or partially reduce, inhibit, interfere with, or modulate the expression and/or activity of one or more checkpoint proteins. In some embodiments, the immune checkpoint inhibitor is a CTLA-4 inhibitor (e.g., an anti-CTLA-4 antigen-binding molecule), a PD-1 inhibitor (e.g., an anti-PD-1 monoclonal antigen-binding molecule) or a PD-L1 inhibitor (e.g., an anti-PD-L1 monoclonal antigen-binding molecule). In some embodiments, the CTLA-4 inhibitor is ipilimumab (YERVOY) or tremelimumab (CP-675,206). In some embodiments, the PD-1 inhibitor is pembrolizumab (KEYTRUDA), nivolumab (OPDIVO), or pidilizumab. In some embodiments, the anti-PD-1 monoclonal antibody is nivolumab or pembrolizumab. In some embodiments, the anti-PD1 antibody is pembrolizumab. In some embodiments, the PD-L1 inhibitor is atezolizumab (TECENTRIQ), avelumab (BAVENCIO), durvalumab (IMFINZI), MEDI4736, or MPDL3280A. In some embodiments, the PD-1 or PD-L1 inhibitor is a small molecule (e.g., those disclosed in US 2018/305313 and WO 2018/195321). In some embodiments, a checkpoint inhibitor can target 4-1BB (e.g., urelumab (BMS-663513) and PF-05082566 (PF-2566)), CD27 (e.g., varlilumab (CDX-1127), CD40 (e.g., CP-870,893), OX40, TIM-3, ICOS, BTLA, A2AR, B7-H3, B7-H4, BTLA, IDO, KIR, LAG3, TIM-3, and VISTA. Additional non-limiting examples of immune checkpoint inhibitors include ulocuplumab, urelumab, PF 05082566, TRX518, varlilumab, CP 870893, PDR001MEDI4736, avelumab, BMS 986016, MGA271, IPH2201, emactuzumab, INCB024360, MEDI6469, galunisertib, BKT140, bavituximab, lirilumab, bevacizumab, MNRP1685A, lambroizumab, CC 90002, BMS-936559, and MGA271.

The term “immune response” refers to any detectable response to a particular substance (such as an antigen) by the immune system of a host mammal, such as innate immune responses (e.g., activation of Toll receptor signaling cascade), cell-mediated immune responses (e.g., responses mediated by lymphocytes T-cells, such as antigen-specific T-cells, and non-specific cells of the immune system), and humoral immune responses (e.g., responses mediated by B cells, such as generation and secretion of antibodies into the plasma, lymph, and/or tissue fluids).

The term “infection” refers to invasion of body tissues by disease-causing microorganisms, their multiplication and the reaction of body tissues to these microorganisms and the toxins they produce. “Infection” includes but are not limited to infections by viruses, prions, bacteria, viroids, parasites, protozoans and fungi. Non-limiting examples of viruses include Retroviridae human immunodeficiency viruses, such as HIV-1 (also referred to as HTLV-III, LAV or HTLV-III/LAV, or HIV-III); and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis, including Norwalk and related viruses); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus, Metapneumovirus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bimaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, VACV, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); and astroviruses. Representative bacteria that are known to be pathogenic include pathogenic Pasteurella species (e.g., Pasteurella multocida), Staphylococcus species (e.g., Staphylococcus aureus), Streptococcus species (e.g., Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae), Neisseria species (e.g., Neisseria gonorrhoeae, Neisseria meningitidis), Escherichia species (e.g., enterotoxigenic E. coli (ETEC), enteropathogenic E. coli (EPEC), enterohemorrhagic E. coli (EHEC), and enteroinvasive E. coli (EIEC)), Bordetella species, Campylobacter species, Legionella species (e.g., Legionella pneumophila), Pseudomonas species, Shigella species, Vibrio species, Yersinia species, Salmonella species, Haemophilus species (e.g., Haemophilus influenzae), Brucella species, Francisella species, Bacteroides species, Clostridium species (e.g., Clostridium difficile, Clostridium perfringens, Clostridium tetani), Mycobacteria species (e.g., M. tuberculosis, M. avium, M. intracellulare, M. kansaii, M. gordonae), Helicobacter pyloris, Borelia burgdorferi, Listeria monocytogenes, Chlamydia trachomatis, Enterococcus species, Bacillus anthracis, Corynebacterium diphtheriae, Erysipelothrix rhusiopathiae, Enterobacter aerogenes, Klebsiella pneumoniae, Fusobacterium nucleatum, Streptobacillus moniliformis, Treponema pallidium, Treponema pertenue, Leptospira, Rickettsia, and Actinomyces israeli. Non-limiting pathogenic fungi include Cryptococcus neoformans, Histoplasma capsulatum, Coccidioides immitis, Blastomyces dermatitidis, Candida albicans, Candida glabrata, Aspergillus fumigata, Aspergillus flavus, and Sporothrix schenckii. Illustrative pathogenic protozoa, helminths, Plasmodium, such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

As used herein, “instructional material” includes a publication, a recording, a diagram, or any other medium of expression which can be used to communicate the usefulness of the compositions and methods of the disclosure. The instructional material of the kit of the disclosure may, for example, be affixed to a container which contains the nucleic acid, peptide, and/or composition of the disclosure or be shipped together with a container which contains the nucleic acid, peptide, and/or composition. Alternatively, the instructional material may be shipped separately from the container with the intention that the instructional material and the compound be used cooperatively by the recipient.

As used herein, “isolated” refers to a cell or a cell population that is removed from its natural environment (such as the peripheral blood) and that is isolated, purified or separated, and is at least about 10%, 205, 30% 40%, 50%, 60%, 70%, 75% free, 80% free, 85% free and preferably at least about 90%, 95%, 96%, 97%, 98%, 99% free, from other cells with which it is naturally present, but which lack the cell surface markers based on which the cells were isolated.

The term “label” when used herein refers to a detectable compound or composition. The label is typically conjugated or fused directly or indirectly to a reagent, such as an antigen-binding molecule, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product. Representative labels include ones that are detectable by for example mass spectrometric, spectroscopic, optical, colourimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; and fluorescent dyes (e.g., fluorophores such as fluorescein, carboxyfluorescein (FAM), tetrachloro-fluorescein, TAMRA, ROX, Cy3, Cy3.5, Cy5, Cy5.5, Texas Red, etc.), bioluminescent moieties, chemiluminescent moieties, alone or in combination with moieties that may suppress or shift emission spectra by fluorescence resonance energy transfer (FRET).

The term “low” or “lo”, as used for example in relation to CD49f⁻, is well known in the art and refers to the expression level of the cell marker of interest (e.g., a cell surface marker such as CD49f), in that the expression level of the cell marker is low by comparison with the expression level of that cell marker in the population of cells being analyzed as a whole. More particularly, the term “lo” refers to a distinct population of cells that expresses the cell marker at a lower level than one or more other distinct population of cells. The term “high” or “hi” or “bright” is well known in the art and refers to the expression level of the cell marker of interest (e.g., a cell surface marker such as CD49f), in that the expression level of the cell marker is high by comparison with the expression level of that cell marker in the population of cells being analyzed as a whole. Generally, cells in the top 2, 3, 4, 5, 6, 7, 8, 9, 10% of the level of expression of a cell marker of interest (e.g., a cell surface marker such as CD49f), as compared to the population of cells as a whole, are designated “hi”, with those falling in the top half of the population categorized as being “+”. Typically, those cells falling below 50% of the level of expression of a cell marker of interest (e.g., a cell surface marker such as CD49f), as compared to the population of cells as a whole, are designated as “lo” cells. Generally, the term “intermediate” or “int” refers to a distinct population of cells that express a cell marker of interest (e.g., a cell surface marker such as CD49f) at a level that is between that expressed by two or more other distinct populations within a sample, for example between a population designated “hi” and a population of cells designated as “lo”. In particular embodiments, “hi” when referring to a positive marker (e.g., a cell surface marker such as CD49f), refers to a level of expression of the cell surface marker on a T-cell (e.g., a memory T-cell such as a CD27⁺ CD28⁺ memory T-cell) that is at least 10%, at least 20%, at least 30%, at least 40%, at least 50%, at least 60%, at least 70%, at least 75%, at least 80%, at least 85%, at least 90%, at least 100% (i.e., 1-fold), at least 2-fold, at least 5-fold, at least 10-fold, at least 100-fold, at least 1000-fold higher than the level of expression of the same marker on a control cell. Essentially any cell that is not a CD49f⁺ T-cell, as that term is used herein, can be used as a control cell. In one embodiment, the control cell is a CD49f⁻ cell, suitably a CD49f⁻ T-cell. In another embodiment, the control cell is a reference value or number related to the level of expression of the marker and obtained from a population of cells that are not CD49f⁺ T-cells (e.g., CD49f⁻ cells, suitably a CD49f⁻ T-cells). In one embodiment, the term “CD49f^{hi}” refers to a level of expression of CD49f on the surface of a T-cell (e.g., a memory T-cell such as a CD27⁺ CD28⁺ memory T-cell) that is at least 1 standard deviation, at least 2 standard deviations, at least 5 standard deviations, at least 10 standard deviations or more above the level of expression of CD49f on the surface of a control cell.

The terms “level of expression” or “expression level” are used interchangeably herein and generally refer to the amount of a biomarker in a sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (e.g., transfer and ribosomal RNAs). The means for determining the level of biomarkers include methods well known to the person skilled in the art, including techniques based on hybridization, amplification, enzymatic elongation or ligation, sequencing, mass spectroscopy, immune assays, flow cytometer or any combination thereof. Not limiting examples include microarray (Agilent, LC Sciences, Affymetrix, febit), next generation sequencing (ABI Solid, Illumina, Oxford Nanopores, Pacific Biosystems, Roche 454, Ion Torrent), qRT-PCR (ABI TaqMan, Qiagen miScript), PCR, color-coded bead assays (Luminex), ligation-based assays (Nanostring, Firefly Bioworks), elongation-based assays (febit MPEA). “Elevated expression”, “elevated expression levels”, or “elevated levels” refers to an increased expression or increased levels of a biomarker in a sample relative to a suitable control, such as a CD49f⁻ and/or CD49f^{lo} immune cell including a CD49f⁻ or CD49f^{lo} T-cell, or an internal control (e.g., housekeeping biomarker). “Reduced expression”, “reduced expression levels”, or “reduced levels” refers to a decreased expression or decreased levels of a biomarker in a sample relative to a suitable control, such as a CD49f⁻ and/or CD49f^{lo} immune cell including a CD49f⁻ or CD49f^{lo} T-cell, or an internal control (e.g., housekeeping biomarker). In some embodiments, reduced expression is little or no expression.

By “likelihood” is meant a measure of whether a T-cell population is (or is not) competent for immunotherapy based on a given mathematical model. An increased likelihood for example may be relative or absolute and may be expressed qualitatively or quantitatively. For instance, an increased likelihood that a T-cell population is competent for immunotherapy may be determined simply by determining the level or concentration of CD49f⁺ T-cells, or subtypes thereof as disclosed for example herein, in the T-cell population, and placing the T-cell population in an “increased likelihood” category in respect of being competent for immunotherapy, based upon previous population studies. The term “likelihood” is also used interchangeably herein with the term “probability”. In some embodiments, “likelihood” is assessed by comparing the level or concentration of CD49f⁺ T-cells, or subtypes thereof as disclosed for example herein, in the T-cell population to one or more preselected or threshold levels. Thresholds may be selected that provide an acceptable ability to predict competence for immunotherapy. In illustrative examples, receiver operating characteristic (ROC) curves are calculated by plotting the value of a variable versus its relative frequency in two populations in which a first T-cell population has a first competence and a second T-cell population has a second competence (called arbitrarily, for example, “incompetent for immunotherapy”, “competent for immunotherapy”, “low competence for immunotherapy”, “high competence for immunotherapy”).

The term “lymphocytes” as used herein refers to cells of the immune system which are a type of white blood cell. Lymphocytes include, but are not limited to, T-cells (cytotoxic and helper T-cells), B-cells and natural killer cells (NK cells). The term “tumor infiltrating lymphocyte” as used herein refers to lymphocytes that are present in a solid tumor. The term “circulating lymphocyte” as used herein refers to lymphocytes that are present in the circulation (e.g., present in blood).

The term “memory T-cell” refers to a T-cell that has previously encountered and responded to a cognate antigen (e.g., a cancer-associated antigen or infectious disease-associated antigen). At a second or later encounter with the cognate antigen the memory T-cell can expand into large numbers of effector T-cells to produce a rapid immune response to the antigen. As used herein, the term “central memory T-cells”, refers to a subgroup or subpopulation of T-cells that have higher expression of genes associated with trafficking to secondary lymphoid organs, which genes include CD62L, CXCR3, CCR7, in comparison to memory effector T-cells. As used herein, the term “stem memory T-cells”, or “stem cell memory T-cells”, refers to a subgroup or subpopulation of T-cells that are capable of self-renewing and generating memory T-cells (e.g., central memory T-cells) and effector T-cells, and express CD27 and lymphoid homing molecules such as CCR7 and CD62L, which are properties important for mediating long-term immunity. By “memory T effector cells” is meant a subset of T-cells including CTL and helper T-cells that have previously encountered and responded to their cognate antigen; thus, the term antigen-experienced T-cell is often applied. Such T-cells can recognize foreign microbes, such as bacteria or viruses, as well as cancer cells. Memory T effector cells have become “experienced” by having encountered antigen during a prior infection, encounter with cancer, or previous vaccination. At a second encounter with the cognate antigen, memory T effector cells can reproduce to mount a faster and stronger immune response than the first time the immune system responded to the microbe. This behavior is utilized in T lymphocyte proliferation assays, which can reveal exposure to specific antigens. In general, after antigen experience, central and effector memory T cells gain expression of CD45RO and lose expression of CD45RA. Thus either CD45RA or CD45RO is used to generally differentiate the naïve from memory populations. CCR7 and CD62L are two other markers that can be used to distinguish central and effector memory T cells. Naïve and central memory cells express CCR7 and CD62L in order to migrate to secondary lymphoid organs. Thus, naïve T cells are generally CD45RA⁺ CD45RO⁻ CCR7⁺ CD62L⁺, central memory T cells are CD45RA⁻ CD45RO⁺ CCR7⁺ CD62L⁺, and effector memory T cells are CD45RA⁻ CD45RO⁺ CCR7⁻ CD62L⁻.

The term “early memory”, as used herein, refers to a CD49f⁺ immune cell, typically a CD49f⁺ T-cell (e.g., a CD49f^{hi} or CD49f^{int} T-cell), that is characterized by expression of any one or more of TCF-1, LEF-1, CD27 and CD28.

As used herein, the term “modified T-cells” refers to T-cells that have been modified by the introduction of a polynucleotide encoding a recombinant or engineered TCR or CAR. Modified T-cells include both genetic and non-genetic modifications (e.g., episomal or extrachromosomal). As used herein, the term “genetically engineered” or “genetically modified” refers to the addition of extra genetic material in the form of DNA or RNA into the total genetic material in a cell. The terms “genetically modified cells” and “modified cells” are used interchangeably.

As used herein, the term “negative for” or “−” when referring to a cell negative for a marker (or the term “does not express”) means that a cell surface marker cannot be detected above background levels on the cell using immunofluorescence microscopy or flow cytometry methods, such as fluorescence activated cell sorting (FACS). Alternatively, the terms “negative” or “does not express” means that expression of the mRNA for an intracellular marker or cell surface marker (e.g., protein, glycoprotein, or polypeptide, among others) cannot be detected above background levels using RT-PCR. The expression level of a cell surface marker or intracellular marker can be compared to the expression level obtained from a negative control (i.e., cells known to lack the marker) or by isotype controls (i.e., a control antibody that has no relevant specificity and only binds non-specifically to cell proteins, lipids or carbohydrates). Thus, a cell that “does not express” a marker appears similar to the negative control for that marker.

The term “package insert” is used herein to refer to instructions customarily included in commercial packages of therapeutic products, that contain information about the indications, usage, dosage, administration, combination therapy, contraindications and/or warnings concerning the use of such therapeutic products.

The terms “patient”, “subject” “recipient” or “treated individual” are used interchangeably herein, to refer broadly to any vertebrate animal that is in need of treatment either to alleviate a disease state or to prevent the occurrence or reoccurrence of a disease state. Suitable vertebrate animals that fall within the scope of the disclosure include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of eliciting an immune response, including an immune response that is predicated at least in part by T-cells having high immune effector function. However, it will be understood that the aforementioned terms do not imply that symptoms are present.

The term “pharmaceutical composition” refers to a preparation which is in such form as to permit the biological activity of the active ingredient(s) to be effective, and which contains no additional components which are unacceptably toxic to a subject to which the composition or formulation would be administered. Such formulations are sterile. “Pharmaceutically acceptable” excipients (vehicles, additives) are those which can reasonably be administered to a subject mammal to provide an effective dose of the active ingredient employed.

The term “phenotype” refers to a trait, or to a class or set of traits displayed by a cell or organism, including for example, morphology, development, biochemical or physiological properties, phenology, behavior, and products of behavior. In some embodiments, a particular phenotype may correlate with a particular developmental stage. In some embodiments, a particular phenotype may correlate with a particular allele or genome. In some embodiments, a particular phenotype may correlate with a particular transcriptome. In some embodiments, a particular phenotype may correlate with a particular epigenome. In some embodiments, a phenotype may be discrete; in some embodiments, a phenotype may be continuous.

The term “positive selection” as used herein refers to selection of a desired cell type by retaining the cells of interest. In some embodiments, positive selection involves the use of an agent to assist in retaining the cells of interest, e.g., use of a positive selection agent such as an antigen-binding molecule that has specific binding affinity for a surface antigen on the desired or target cell. In some embodiments, positive selection can occur in the absence of a positive selection agent, e.g., in a “touch-free” or closed system, for example, where positive selection of a target cell type is based on any of cell size, density and/or morphology of the target cell type. The term “negative selection” as used herein refers to selection of undesired or non-target cells for depletion or discarding, thereby retaining (and thus enriching) the desired target cell type. In some embodiments, negative selection involves the use of an agent to assist in selecting undesirable cells for discarding, e.g., use of a negative selection agent such as an antigen-binding molecule that has specific binding affinity for a surface antigen on unwanted or non-target cells. In some embodiments, negative selection does not involve a negative selection agent. In some embodiments, negative selection can occur in the absence of a negative selection agent, e.g., in a “touch-free” or closed system, for example, where negative selection of an undesired (non-target) cell type to be discarded is based on any of cell size, density and/or morphology of the undesired (non-target) cell type.

As used herein, the term “positive for” or “+” when referring to a cell positive for a marker (e.g., CD49f positive or CD49f⁺) means that a cell surface marker is detectable above background levels on the cell using immunofluorescence microscopy or flow cytometry methods, such as fluorescence activated cell sorting (FACS). Alternatively, the terms “positive for” or “expresses a marker” means that expression of mRNA encoding a cell surface or intracellular marker is detectable above background levels using RT-PCR. The expression level of a cell surface marker or intracellular marker can be compared to the expression level obtained from a negative control (i.e., cells known to lack the marker) or by isotype controls (i.e., a control antibody that has no relevant specificity and only binds non-specifically to cell proteins, lipids or carbohydrates). Thus, a cell that “expresses” a marker (or is “positive for a marker”) has an expression level detectable above the expression level determined for the negative control for that marker.

“Potent T-cells” and “young T-cells” are used interchangeably herein in some embodiments to refer to T-cell phenotypes wherein the T-cell is capable of proliferation and suitably with reduced or little differentiation. In particular embodiments, the potent T-cell has a early memory phenotype. In various embodiments, the manufacturing processes disclosed herein produce young T-cells; in some embodiments cells wherein T-cell proliferation has been uncoupled from T-cell differentiation during T-cell stimulation, activation, and expansion. Without wishing to be bound by any particular theory, the potent T-cells produced by the processes of the present disclosure possess greater efficacy for immunotherapy, in particular adoptive cell therapy. In certain embodiments, young T-cells are positive or express intermediate and/or high levels of CD49f, and one or more of, or all of the following biological markers: CD95, CD45RA, CCR7, CD28, CD27, TCF-1, LEF-1 and one or both of CD8 and CD4. In some embodiments, the young T-cells are negative or lack expression of: a terminal differentiation biomarker such as CD57; an NK biomarker such as CD244 and CD160; an immune checkpoint molecule such as PD-1, CTLA4, TIM3, and LAG3.

The terms “proliferation” and “proliferate” are used interchangeably herein to refer to the expansion of cells by division, either symmetric or asymmetric division of cells, including repeated division, of cells into two daughter cells. “Increased proliferation” occurs when there is an increase in the number of cells in a treated sample compared to cells in a non-treated sample. The term “proliferative potential” refers to the ability of a cell to proliferate. an increase in cell division. In particular embodiments, “proliferation” refers to the symmetric or asymmetric division of T-cells.

As used herein, the term “responsiveness” or “responsive” when used in connection with a treatment such as an immunotherapy (e.g., adoptive cell therapy) refers to the effectiveness of the treatment in lessening or decreasing the symptoms of the disease being treated. For example, a cancer patient is responsive to treatment with an immune cell-containing population of the present disclosure if the treatment effectively inhibits the cancer growth, or arrests development of the cancer, causes regression of the cancer, or delays or minimizes one or more symptoms associated with the presence of the cancer in the patient. Alternatively, a patient having an infectious disease is responsive to treatment with an immune cell-containing population of the present disclosure if the treatment effectively inhibits the infection, or arrests development of the infection, causes regression of the infection, or delays or minimizes one or more symptoms associated with the presence of the infection in the patient.

The term “resting” is well known in the art and refers to an immune cell or a population of cells that does not proliferate, does not produce cytokines and that does not express conventional immune cell activation molecules at the surface such as CD25.

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., feces), tears, sweat, sebum, nipple aspirate, ductal lavage, tumor exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. Suitably, the sample is readily obtainable by minimally invasive methods, allowing the removal or isolation of the sample from the subject. In certain embodiments, the sample contains blood, especially peripheral blood, or a fraction or extract thereof. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises leukocytes including peripheral blood mononuclear cells (PBMC). In some embodiments, the sample comprises stored cells or cultured cells.

As used herein, the term “stem-like” refers to a state in which cells acquire characteristics of stem cells or progenitor cells, share important elements of the gene expression profile of stem cells progenitor cells. Stem-like cells may be somatic cells undergoing induction to a less mature state, such as increasing expression of pluripotency genes such as, but not limited to, Sox2 and Oct4. Stem-like cells also refers to cells that have undergone some de-differentiation or are in a meta-stable state from which they can alter their terminal differentiation.

By “stimulation”, is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

Various methodologies of the instant disclosure include a step that involves comparing a value, level, feature, characteristic, property, etc. to a “suitable control,” referred to interchangeably herein as an “appropriate control,” a “control sample” or a “reference.” A “suitable control”, “appropriate control”, “control sample” or a “reference” is any control or standard familiar to one of ordinary skill in the art useful for comparison purposes. In some embodiments, a “suitable control” or “appropriate control” is a value, level, feature, characteristic, property, etc., determined in a cell, organ, or patient, e.g., a control cell, cell population, organ, or patient, exhibiting, for example, a particular profile of immune properties (e.g., a profile comprising one or more of an early memory phenotype, a stem-like phenotype, increased proliferative potential, increased survival, increased immune effector function, decreased immune effector dysfunction and increased responsiveness in immunotherapy; or a profile lacking one or more of an early memory phenotype, a stem-like phenotype, increased proliferative potential, increased survival, increased immune effector function, decreased immune effector dysfunction and increased responsiveness in immunotherapy). In other embodiments, a “suitable control” or “appropriate control” is a value, level, feature, characteristic, property, ratio, etc. (e.g., biomarker levels that correlate to a particular immune effector property profile) determined prior to CD49f enrichment. In some embodiments, a transcription rate, mRNA level, translation rate, protein level/ratio, biological activity, cellular characteristic or property, genotype, phenotype, etc., can be determined prior to, during, or after CD49f enrichment. In a further embodiment, a “suitable control,” “appropriate control” or a “reference” is a predefined value, level, feature, characteristic, property, ratio, etc. A “suitable control” can be a pattern of levels/ratios of one or more biomarkers of the present disclosure that correlates to a particular profile of immune properties (e.g., a profile comprising one or more of an early memory phenotype, a stem-like phenotype, increased proliferative potential, increased survival, increased immune effector function, decreased immune effector dysfunction and increased responsiveness in immunotherapy; or a profile lacking one or more of an early memory phenotype, a stem-like phenotype, increased proliferative potential, increased survival, increased immune effector function, decreased immune effector dysfunction and increased responsiveness in immunotherapy), to which a T-cell population sample can be compared. The immune cell population sample (e.g., a T-cell population sample) can also be compared to a negative control. Such reference levels may also be tailored to specific techniques that are used to measure levels of biomarkers in biological samples (e.g., LC-MS, GC-MS, ELISA, PCR, etc.), where the levels of biomarkers may differ based on the specific technique that is used.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

As used herein, the terms “T-cell” or “T lymphocyte” are art-recognized and are intended to include thymocytes, naïve T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4⁺ T-cell), a cytotoxic T-cell (CTL; CD8⁺ T-cell), a tumor infiltrating cytotoxic T-cell (TIL; CD8⁺ T-cell), CD4⁺ CD8⁺ T-cell, CD4-CD8⁻ T-cell, an αβ T-cell expressing T-cell receptor (TCR) α and β chains, and a γδ T-cell expressing TCR γ and δ chains, or any other subset of T-cells. Other illustrative populations of T-cells suitable for use in particular embodiments include memory T-cells, suitably early memory T-cells. The term “T-cell” includes a precursor cell of a T-cell in which differentiation into a T-cell is directed. The term “T-cell” includes within its scope natural T-cells (e.g., isolated from an organism, e.g., a mammal, e.g., a human, e.g., a subject), T-cells grown ex vivo, and genetically engineered T-cells. The term T-cell also encompasses T-cells comprising a T-cell receptor (e.g., a natural TCR, or a recombinant TCR) and to T-cells comprising an artificial T-cell receptor (e.g., CAR-T cells).

A “T-cell dysfunctional disorder” is a disorder or condition of T-cells characterized by decreased responsiveness to antigenic stimulation. In a particular embodiment, a T-cell dysfunctional disorder is a disorder that is specifically associated with inappropriate increased signaling through an immune checkpoint protein (e.g., PD-1, CTLA-4, etc.). In another embodiment, a T-cell dysfunctional disorder is one in which T-cells are anergic or have decreased ability to secrete cytokines, proliferate, or execute cytolytic activity. In a specific aspect, the decreased responsiveness results in ineffective control of a pathogen or tumor expressing an immunogen. Examples of T-cell dysfunctional disorders characterized by T-cell dysfunction include unresolved acute infection, chronic infection and tumor immunity.

The term “T-cell exhaustion” refers to a state of T-cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T-cells. Exhaustion prevents optimal control of infection and tumors. Exhaustion can result from both extrinsic negative regulatory pathways (e.g., immunoregulatory cytokines) as well as cell intrinsic negative regulatory (co-stimulatory) pathways (PD-1, B7-H3, B7-H4, etc.). In specific embodiments, T-cell exhaustion is characterized by an elevated expression level of Eomesodermin (EOMES) and a decreased expression level of TBET, relative to an activated T-cell.

As used herein, the terms “T-cell manufacturing” or “process of manufacturing T-cells” and the like refer to the process of producing a therapeutic population of T-cells, which manufacturing process may comprise one or more of, or all of the following steps: CD49f enrichment, harvesting, stimulation, activation, and expansion.

The term “transduce” or “transduction” as it is applied to the production of recombinant antigen receptor cells or chimeric antigen receptor cells refers to the process whereby a foreign nucleotide sequence is introduced into a cell. In some embodiments, this transduction is done via a vector.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with a T-cell dysfunctional disorder are mitigated or eliminated, including, but are not limited to, reducing the proliferation of (or destroying) cancerous cells, reducing pathogen infection, decreasing symptoms resulting from the disease, increasing the quality of life of those suffering from the disease, decreasing the dose of other medications required to treat the disease, and/or prolonging survival of individuals.

As used herein the term “vector” refers to an agent that can transduce, transfect, transform or infect a cell, thereby causing the cell to express nucleic acids and/or proteins other than those native to the cell, or in a manner not native to the cell. A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

Each embodiment described herein is to be applied mutatis mutandis to each and every embodiment unless specifically stated otherwise.

### 2. Abbreviations

The following abbreviations are used throughout the application:
EBV=Epstein-Barr Virus
CMV=Cytomegalovirus
FACS=fluorescence activated cell sorting
HLA=human leukocyte antigen
i.v.=Intravenous
ICOS=inducible T-cell costimulatory
IFNγ=interferon gamma
IL-2=interleukin-2
LAG-3=lymphocyte-activation gene 3
LCL=lymphoblastic cell line
PD-1=programmed cell death protein 1
qPCR=quantitative polymerase chain reaction
TCR=T-cell receptor
TCRβ=TCR beta chain
TIM-3=T-cell immunoglobulin and mucin-domain containing-3
TNF=tumor necrosis factor

### 3. Processes for Manufacturing T-Cell Populations with Enhanced Properties for Immunotherapy

The present disclosure generally relates to processes for manufacturing T-cell populations with enhanced or superior immune properties, e.g., one or more of an early memory phenotype, a stem-like phenotype, increased proliferative potential, increased survival and increased persistence in vivo, decreased differentiation, increased immune effector function, decreased immune effector dysfunction and increased responsiveness in immunotherapy, compared to existing T-cell populations in the art. Notably, the T-cell populations disclosed herein comprise T-cells that comprise characteristics of young or early memory T-cell populations, including being capable of multiple rounds of proliferation, suitably with little or reduced T-cell differentiation, as compared with T-cell populations in the art.

The present inventors have surprisingly and unexpectedly discovered that enriching T-cell populations for CD49f⁺ cells produces T-cell populations with enhanced or superior immune properties as broadly described above. In particular embodiments, an engineered T-cell population is produced by the processes disclosed herein, which may further increase the efficacy of an adoptive cell therapy. The CD49f⁺ T-cell enriched T-cell populations disclosed herein are useful in treating or inhibiting the development of numerous conditions including, but not limited to cancer, infectious disease, autoimmune disease, inflammatory disease, and immunodeficiency.

Thus, disclosed herein is a process of manufacturing a T-cell population with enhanced or superior properties as broadly described above and elsewhere herein, which process comprises or consists essentially of: isolating or selecting from a sample containing T-cells a T-cell population comprising CD49f⁺ T-cells, wherein the CD49f⁺ T-cells constitute at least 1% (including at least 2% to 99% and all integer percentages therebetween) of the T-cells in the population, or enriching a sample containing T-cells for CD49f⁺ T-cells, to thereby manufacture a T-cell population comprising T-cells with enhanced immune properties. The manufactured T-cell populations disclosed herein are suitably enriched in developmentally potent T-cells that express CD49f, and one or more, or all, of the following biomarkers: CD95, CD45RA, CCR7, CD28, CD27, TCF-1, LEF-1 and one or both of CD8 and CD4

3.1 Populations of Cells

The T-cell-containing sample can be obtained from any suitable source. For example, the T-cell-containing sample can be an isolated cell sample, including a primary cell sample such as a primary human cell sample. The isolated cell sample typically includes a population of blood or blood-derived cells, such as hematopoietic cells, leukocytes (white blood cells), peripheral blood mononuclear cells (PBMCs), and/or cells of the immune system, e.g., cells of the innate or adaptive immunity, such as myeloid or lymphoid cells, e.g., lymphocytes, typically T-cells and/or NK cells. In some embodiments, the sample is an apheresis or leukapheresis sample. In some embodiments, the enrichment can include a negative selection (i.e., depletion) of cells from the sample, for example, cells expressing non-T-cell markers, such as myeloid or B cell markers, for example, negative selection for cells expressing CD14, CD19, CD56, CD20, CD11b, and/or CD16. T-cell-containing samples that can be enriched for CD49f⁺ T-cells include populations of unfractionated T-cells, unfractionated CD4⁺ T-cells, unfractionated CD8⁺ T-cells, and sub-populations of CD4⁺ and/or CD8⁺ T-cells, including subpopulations of T-cells generated by enrichment for or depletion of cells of a particular sub-type or based on a particular surface marker expression profile.

Among the sub-types and subpopulations of T-cells that can be contained in a T-cell-containing sample are naïve T (TN) cells, effector T-cells (TEFF), memory T-cells and sub-types thereof, such as stem cell memory T (TSCM), central memory T (TCM), effector memory T (TEM), or terminally differentiated effector memory T-cells, tumor-infiltrating lymphocytes (TIL), immature T-cells, mature T-cells, helper T-cells, cytotoxic T-cells, mucosa-associated invariant T (MAIT) cells, naturally occurring and adaptive regulatory T (Treg) cells, helper T-cells, such as TH₁ cells, TH₂ cells, TH₃ cells, TH₁₇ cells, TH₉ cells, TH₂₂ cells, follicular helper T-cells, αβ T-cells, and γδ T-cells. In some of the same and other embodiments, the T-cell-containing sample contains any one or more of NK cells, monocytes, granulocytes, e.g., myeloid cells, macrophages, neutrophils, dendritic cells, mast cells, eosinophils, and/or basophils. In specific embodiments, the T-cell-containing sample contains central memory (TCM) cells, which suitably have an early memory phenotype.

3.2 Samples

The T-cell-containing sample is typically a biological sample, e.g., one obtained from or derived from a subject, such as one having a particular disease or condition or in need of a cell therapy or to which cell therapy will be administered. In some embodiments, the subject is a human, such as a subject who is a patient in need of a particular therapeutic intervention, such as the adoptive cell therapy for which cells are being isolated, enriched, selected, processed, and/or engineered. Accordingly, the cells in some embodiments are primary cells, e.g., primary human cells. The samples may include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain embodiments, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, cultured cells, including T-cell lines, are used as the T-cell-containing sample. The T-cell-containing sample in some embodiments is obtained from a xenogeneic source, for example, from mouse, rat, non-human primate, and pig.

3.3 Cell Processing, Preparation and Non-Affinity-Based Separation

In some embodiments, isolation of the T-cell-containing sample includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T-cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In illustrative examples, a washing step is accomplished using a semi-automated “flow-through” centrifuge (e.g., the Cobe 2991 cell processor, Baxter) according to the manufacturer's instructions. In other illustrative examples, a washing step is accomplished by tangential flow filtration (TFF) according to the manufacturer's instructions. In some embodiments, the cells are resuspended in a variety of biocompatible buffers after washing, such as, for example, Ca²⁺Mg²⁺ free PBS. In certain embodiments, components of a blood cell sample are removed and the cells directly resuspended in culture media.

In some embodiments, the manufacturing processes include density-based cell separation methods, such as the preparation of white blood cells from peripheral blood by lysing the red blood cells and centrifugation through a Percoll or Ficoll gradient.

3.4 Separation Based on Affinity and/or Marker Profile

The manufacturing processes disclosed herein include positive selection for cells that are CD49f⁺, and optionally positive or negative selection of other cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some embodiments includes separation of cells and cell populations based on the cells' expression or expression level of one or more markers, typically cell surface markers, for example, by incubation with an antigen-binding molecule or binding partner that specifically binds to such markers, followed generally by washing steps and separation of cells having bound the antigen-binding molecule or binding partner, from those cells having not bound to the antigen-binding molecule or binding partner.

Such separation steps can be based on positive selection, in which the cells having bound the reagents are retained for further use, and/or negative selection, in which the cells having not bound to the antigen-binding molecule or binding partner are retained. In some examples, both fractions are retained for further use. In some embodiments, negative selection can be particularly useful where no antigen-binding molecule or binding partner is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those that are positive for a marker (e.g., CD49f), refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells. For example, a selection of CD49f⁺ cells enriches for those cells in a population, but also can contain some residual or small percentage of other non-selected cells still being present in the enriched population.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antigen-binding molecules or binding partners, each specific for a marker targeted for negative selection. Likewise, multiple cell types can simultaneously be positively selected by incubating cells with a plurality of antibodies or binding partners expressed on the various cell types.

For example, in some embodiments, specific subpopulations of T-cells, such as cells positive or expressing high and/or intermediate levels of one or more surface markers, e.g., CD49f, and optionally one or more of CD45RA, CCR7, CD28, CD27 and one or both of CD8 and CD4, are isolated by positive or negative selection techniques. For example, CD49f⁺ T-cells can be positively selected using an anti-CD49f antigen-binding molecule optionally in combination with one or more of an anti-CD45RA antigen-binding molecule, an anti-CCR7 antigen-binding molecule, an anti-CD28 antigen-binding molecule, an anti-CD27 antigen-binding molecule, an anti-CD95 antigen-binding molecule, an anti-CD8 antigen-binding molecule, and an anti-CD4 antigen-binding molecule. In specific embodiments, respective antigen-binding molecules are conjugated to a magnetic bead (e.g., MILTENYL MACS MICROBEAD or DYNABEAD).

In some embodiments, isolation is carried out by enrichment for a particular cell population by positive selection, or depletion of a particular cell population, by negative selection. In some embodiments, positive or negative selection is accomplished by incubating cells with one or more antigen-binding molecules or binding partners that specifically bind to one or more surface markers expressed or expressed at a relatively higher level (marker^{hi}) on the positively or negatively selected cells, respectively.

In illustrative examples of this type, T-cells are separated from non-T-cells by negative selection of markers expressed on non-T cells, such as B cells, monocytes, or other white blood cells, such as CD14. In some embodiments, the productions processes include isolation, selection and/or enrichment of CD49f⁺ cells before or after the negative selection of markers expressed on non-T cells.

In some embodiments, a subpopulation of T-cells is subjected to positive selection for CD49f⁺ cells (e.g., CD49f^{hi} and/or CD49f^{int} cells) and to selection for CD4⁺ cells and/or CD8⁺ cells. In one example, to enrich for CD4⁺ cells by negative selection, an antigen-binding molecule cocktail typically includes antigen-binding molecules to CD14, CD20, CD11b, CD16 and HLA-DR. In one example, enriching for CD8⁺ cells by negative selection is carried out by depletion of cells expressing CD14 and/or CD45RA. In some embodiments, a CD4⁺ or CD8⁺ selection step, such as positive selection for CD4 and positive selection for CD8, is used to separate CD4⁺ helper and CD8⁺ cytotoxic T-cells. Such selections may be carried out simultaneously, or sequentially in either order. The positive selection for CD49f⁺ cells can occur before, after or simultaneously with the selection for CD4⁺ cells and/or CD8⁺ cells.

In some embodiments, the manufacturing processes, before or after positive selection for CD49f⁺ cells (e.g., CD49f^{hi} and/or CD49f^{int} cells), include a first positive selection for CD4⁺ cells in which the non-selected cells (CD4⁻ cells) from the first selection are used as the source of cells for a second positive selection to enrich for CD8⁺ cells. In some aspects, the processes include a first positive selection for CD8⁺ cells in which the non-selected cells (CD8⁻ cells) from the first selection are used as the source of cells for a second position selection to enrich for CD4⁺ cells. Such CD4⁺ and CD8⁺ populations can be further sorted into sub-populations by positive or negative selection for markers expressed or expressed to a relatively higher degree on one or more naïve, memory, and/or effector T-cell subpopulations. In non-limiting examples of this type, CD4⁺ cells are further enriched for or depleted of naïve, central memory, effector memory and/or central memory stem cells, such as by positive or negative selection based on surface antigens associated with the respective population. CD4⁺ T helper cells are sorted into naïve, central memory, and effector cells by identifying cell populations that have cell surface antigens. CD4⁺ lymphocytes can be obtained by standard methods. In some embodiments, naïve CD4⁺ T lymphocytes are CD45RO−, CD45RA⁺, CD62L⁺, CD4⁺ T-cells. In some embodiments, central memory CD4⁺ cells are CD62L⁺ and CD45RO⁺. In some embodiments, effector CD4⁺ cells are CD62L- and CD45RO⁺. In non-limiting examples, CD8⁺ cells are further enriched for or depleted of naïve, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T-cells and CD4⁺ T-cells further enhances efficacy.

In some embodiments, memory T-cells are present in both CD62L⁺ and CD62L⁻ subsets of CD8⁺ peripheral blood lymphocytes. PBMC can be enriched for or depleted of CD62L⁻ CD8⁺ and/or CD62L⁺ CD8⁺ fractions, such as using anti-CD8 and anti-CD62L antigen-binding molecules.

In some embodiments, the enrichment for central memory T (T_{CM}) cells is based on positive or high surface expression of CD45RO, CD62L, CCR7, CD28, CD95, CD3, CD27 and/or CD127; in some aspects, it is based on negative selection for cells expressing or highly expressing CD45RA and/or CD57.

In some embodiments, the disclosed herein manufacturing processes include isolation, selection and/or enrichment of CD49f⁺ cells (e.g., CD49f^{hi} and/or CD49f^{int} cells) CD8⁺ cells from a sample, such as by positive selection based on surface expression of CD49f and CD8. In some embodiments, the manufacturing processes can further include enriching for central memory T (T_{CM}) cells. For example, the enriched CD49f⁺ CD8⁺ cells can be further enriched for central memory T (T_{CM}) cells by selecting for one or more markers expressed on central memory T (T_{CM}) cells, such as one or more of CD95, CD45RO, CD62L, CCR7, CD28, CD3, CD27 and/or CD 127. The selection can be performed prior to or subsequent to isolation, selection and/or enrichment of CD49f⁺ CD4⁺ cells. Such selections in some embodiments can be carried out simultaneously, or sequentially in either order.

In some embodiments, the manufacturing processes, before or after positive selection for CD49f⁺ cells (e.g., CD49f^{hi} and/or CD49f^{int} cells), include a first positive selection for CD4⁺ cells in which the non-selected cells (CD4⁻ cells) from the first selection are used as the source of cells for a second selection to enrich for CD8⁺ cells, and the enriched or selected CD8⁺ cells are used in a third selection to further enrich for cells expressing one or more markers expressed on central memory T (T_{CM}) cells, such as by one or more additional selections to enrich for any one or more of CD95⁺, CD45RO⁺, CD62L⁺, CCR7⁺, CD28⁺, CD3⁺, CD27⁺ and CD127⁺ cells. In some embodiments, the manufacturing processes include a first positive selection for CD8⁺ cells in which the non-selected cells (CD8⁻ cells) from the first selection are used as the source of cells for the second selection to enrich for CD4⁺ cells, and the enriched or selected CD8⁺ cells from the first selection also are used in a third selection to further enrich for cells expressing one or more markers expressed on central memory T (T_{CM}) cells, such as by a third selection to enrich for CD95⁺ CD45RO⁺, CD62L⁺, CCR7⁺, CD28⁺, CD3⁺, CD27⁺ and/or CD127⁺ cells.

In some embodiments, before or after positive selection for CD49f⁺ cells (e.g., CD49f^{hi} and/or CD49f^{int} cells), isolation of a CD8⁺ population enriched for T_{CM} cells is carried out by depletion of cells expressing CD4, CD14, CD45RA, and positive selection or enrichment for cells expressing CD62L. In non-limiting examples, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4⁻ based separation are retained and used in subsequent steps of the manufacturing processes, optionally following one or more further positive or negative selection steps.

In a particular example, a sample of PBMCs or other white blood cell sample, before or after positive selection for CD49f⁺ cells (e.g., CD49f^{hi} and/or CD49f^{int} cells), is subjected to selection of CD4⁺ cells, where both the negative and positive fractions are retained. The negative fraction then is subjected to negative selection based on expression of CD14 and CD45RA or CD19, and positive selection based on a marker characteristic of central memory T-cells, such as CD62L or CCR7, where the positive and negative selections are carried out in either order.

In some embodiments, the product ion processes of isolating, selecting and/or enriching for cells, such as by positive or negative selection based on the expression of a cell surface marker or markers, for example by any of the processes described above, can include immunoaffinity-based selections. In some embodiments, the immunoaffinity-based selections include contacting a sample containing cells, such as primary human T-cells containing CD49f⁺ cells (e.g., CD49f^{hi} and/or CD49f^{int} cells), which suitable express one or both of CD4 and CD8, with an antigen-binding molecule or binding partner that specifically binds to the cell surface marker or markers. In some embodiments, the antigen-binding molecule or binding partner is bound to a solid support or matrix, such as a sphere or bead, for example microbeads, nanobeads, including agarose, magnetic bead or paramagnetic beads, to allow for separation of cells for positive and/or negative selection. In some embodiments, the spheres or beads can be packed into a column to effect immunoaffinity chromatography, in which a sample containing cells, such as primary T-cells, including primary human T-cells, containing CD49f⁺ cells (e.g., CD49f^{hi} and/or CD49f^{int} cells), which suitable express one or both of CD4 and CD8, is contacted with the matrix of the column and subsequently eluted or released therefrom.

3.4.1 Immunoaffinity Beads

For example, in some embodiments, the cells and cell populations are separated or isolated using immunomagnetic (or affinity-magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher Humana Press Inc., Totowa, N.J.).

In representative examples, the sample or composition of cells to be separated is incubated with small, magnetizable or magnetically responsive material, such as magnetically responsive particles or microparticles, such as paramagnetic beads. The magnetically responsive material, e.g., particle, generally is directly or indirectly attached to an antigen-binding molecule or binding partner that specifically binds to a marker, e.g., surface marker, present on the cell, cells, or population of cells that it is desired to separate, e.g., that it is desired to negatively or positively select. Such beads are known and are commercially available from a variety of sources including, in some aspects, DYNABEADS (Life Technologies, Carlsbad, Calif.), MACS beads (Miltenyi Biotec, San Diego, Calif.) or STREPTAMER bead reagents (IBA, Germany).

In some embodiments, the magnetic particle or bead comprises a magnetically responsive material bound to a specific binding member, such as an antigen-binding molecule or other binding partner. There are many well-known magnetically responsive materials used in magnetic separation methods. Suitable magnetic particles include those described in Molday, U.S. Pat. No. 4,452,773, and in European Patent Specification EP 452342 B. Colloidal sized particles, such as those described in Owen U.S. Pat. No. 4,795,698, and Liberti et al., U.S. Pat. No. 5,200,084 are other examples.

The incubation generally is carried out under conditions whereby the antigen-binding molecules or binding partners, or molecules, such as secondary antigen-binding molecules or other reagents, which specifically bind to such antigen-binding molecules or binding partners, which are attached to the magnetic particle or bead, specifically bind to cell surface molecules if present on cells within the sample.

In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some embodiments, a combination of positive and negative selection is performed during the same selection step, where the positive and negative fractions are retained and further processed or subject to further separation steps.

In certain embodiments, the magnetically responsive particles are coated in primary antigen-binding molecules or other binding partners, secondary antigen-binding molecules, lectins, enzymes, or streptavidin. In certain embodiments, the magnetic particles are attached to cells via a coating of primary antigen-binding molecules specific for one or more markers. In certain embodiments, the cells, rather than the beads, are labeled with a primary antigen-binding molecule or binding partner, and then cell-type specific secondary antigen-binding molecule- or other binding partner (e.g., streptavidin)-coated magnetic particles, are added. In certain embodiments, streptavidin-coated magnetic particles are used in conjunction with biotinylated primary or secondary antigen-binding molecules.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antigen-binding molecules, magnetizable particles or antigen-binding molecules conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labeled and depleted from the heterogeneous population of cells.

In some embodiments, the affinity-based selection employs STREPTAMERS, which are magnetic beads, such as nanobeads or microbeads, for example 1-2 μM that, in some aspects, are conjugated to a binding partner immunoaffinity reagent, such as an antigen-binding molecule via a streptavidin mutant (also commonly referred to as a mutein), e.g. STREP-TACTIN or STREP-TACTIN XT (see e.g. U.S. Pat. No. 6,103,493, International Published PCT Appl. Nos. WO/2013011011, WO 2014/076277). In some embodiments, the streptavidin mutant is functionalized, coated and/or immobilized on the bead. The term “streptavidin mutein”, “streptavidin mutant” or variations thereof, refers to a streptavidin protein that contains one or more amino acid differences compared to an unmodified or wild type streptavidin,

In some embodiments, the streptavidin mutein is a multimer. Multimers can be generated using any methods known in the art, such as any described in published U.S. Patent Application No. US2004/0082012. In some embodiments, oligomers or polymers of muteins can be prepared by the introduction of carboxyl residues into a polysaccharide, e.g. dextran. In some aspects, streptavidin muteins then are coupled via primary amino groups of internal lysine residues and/or the free N-terminus to the carboxyl groups in the dextran backbone using conventional carbodiimide chemistry in a second step. In some embodiments, the coupling reaction is performed at a molar ratio of about 60 moles streptavidin mutant per mole of dextran. In some embodiments, oligomers or polymers of can also be obtained by crosslinking via bifunctional linkers, such as glutardialdehyde or by other methods known in the art.

In some aspects an immunoaffinity bead, such as a STREPTAMER or other immunoaffinity bead, can contain an antigen-binding molecule (e.g., a monoclonal antibody) produced by or derived from a hybridoma as follows: MAB13501 (αCD49f), OKT3 (αCD3), 13B8.2 (αCD4), OKT8 (αCD8), FRT5 (αCD25), DREG56 (αCD62L), MEM56 (αCD45RA). In some embodiments, any of the above antigen-binding molecules can contain one or more mutations within the framework of heavy and light chain variable regions without targeting the highly variable CDR regions. In some embodiments, an antigen-binding fragment, such as a Fab fragment or scFv molecule, can be generated from such antigen-binding molecules using methods known in the art, such as, in some aspects, amplification of hypervariable sequences of heavy and light chains and cloning to allow combination with sequences coding for an appropriate constant domain. In some embodiments, the constant domain is of human subclass IgG 1/κ. Such antigen-binding molecules can be carboxy-terminally fused with a peptide streptavidin binding molecule.

In some embodiments, the antigen-binding molecule specifically that binds to a cell surface marker associated with or coated on a bead or other surface is a full-length antibody or is an antigen-binding fragment thereof, including a (Fab) fragments, F(ab′)₂ fragments, Fab′ fragments, Fv fragments, variable heavy chain (V_{H}) regions capable of specifically binding the antigen, single chain antibody fragments, including single chain variable fragments (scFv), and single domain antibodies (e.g., sdAb, sdFv, nanobody) fragments. In some embodiments, the antigen-binding molecule is a Fab fragment or scFv molecule. In some embodiments, the antigen-binding molecule can be monovalent, bivalent or multivalent. In some embodiments, the antigen-binding molecule, such as a Fab, is a multimer. In some embodiments, the antigen-binding molecule, such as a Fab multimer, forms a multivalent complex with the cell surface marker.

3.4.2 Immunoaffinity Chromatography

In some embodiments, the affinity-based selection employs immunoaffinity chromatography. Immunoaffinity chromatography methods include, in some aspects, one or more chromatography matrix as described in U.S. Published Patent Appl. No. US2015/0024411. In some embodiments, the chromatographic method is a fluid chromatography, typically a liquid chromatography. In some embodiments, the chromatography can be carried out in a flow through mode in which a fluid sample containing the cells to be isolated is applied, for example, by gravity flow or by a pump on one end of a column containing the chromatography matrix and in which the fluid sample exits the column at the other end of the column. In addition, in some aspects, the chromatography can be carried out in an “up and down” mode in which a fluid sample containing the cells to be isolated is applied, for example, by a pipette on one end of a column containing the chromatography matrix packed within a pipette tip and in which the fluid sample enters and exits the chromatography matrix/pipette tip at the other end of the column. In some embodiments, the chromatography can also be carried out in a batch mode in which the chromatography material (stationary phase) is incubated with the sample that contains the cells, for example, under shaking, rotating or repeated contacting and removal of the fluid sample, for example, by means of a pipette.

In some embodiments, the chromatography matrix is a stationary phase. In some embodiments, the chromatography is column chromatography. In some embodiments, any suitable chromatography material can be used. In some embodiments, the chromatography matrix has the form of a solid or semi-solid phase. In some embodiments, the chromatography matrix can include a polymeric resin or a metal oxide or a metalloid oxide. In some embodiments, the chromatography matrix is a non-magnetic material or non-magnetizable material. In some embodiments, the chromatography matrix is a derivatized silica or a crosslinked gel, such as in the form of a natural polymer, for example a polysaccharide. In some embodiments, the chromatography matrix is an agarose gel. Agarose gel for use in a chromatography matrix are known in the art and include, in some aspects, SUPERFLOW agarose or a SEPHAROSE material such as SUPERFLOW SEPHAROSE, which are commercially available in different bead and pore sizes. In some embodiments, the chromatography matrix is a particular cross-linked agarose matrix to which dextran is covalently bonded, such as any known in the art, for example in some aspects, SEPHADEX, SUPERDEX or SEPHACRYL, which are available in different bead and pore sizes.

In some embodiments, a chromatography matrix is made of a synthetic polymer, such as polyacrylamide, a styrene-divinylbenzene gel, a copolymer of an acrylate and a diol or of an acrylamide and a diol, a co-polymer of a polysaccharide and agarose, e.g. a polyacrylamide/agarose composite, a polysaccharide and N,N′-methylenebisacrylamide, or a derivatized silica coupled to a synthetic or natural polymer.

In some embodiments, the chromatography matrix, such as agarose beads or other matrix, has a size of at least or about at least 50 μm, 60 μm, 70 μm, 80 μm, 90 μm, 100 μm, 120 μm or 150 μm or more. The exclusion limit of the size exclusion chromatography matrix is selected to be below the maximal width of the target cell in a sample, e.g. T-cells. In some embodiments, the volume of the matrix is at least 0.5 mL, 1 mL, 1.5 mL, 2 mL, 3 mL, 4 mL, 5 mL, 6 mL, 7 mL, 8 mL, 9 mL, 10 mL or more. In some embodiments, the chromatography matrix is packed into a column.

In some embodiments, the chromatography matrix, which is an immunoaffinity chromatography matrix, includes an affinity reagent, such as an antigen-binding molecule (e.g., an Fab, scFv, or immunoglobulin) immobilized thereto. The antigen-binding molecule can be any as described above, including, in some aspects, known antigen-binding molecules in the art, antigen-binding molecules having a particular k_{Off} rate and/or antigen-binding molecules having a particular dissociation constant (Ka).

In some embodiments, the affinity reagent, such as an antigen-binding molecule (e.g., an Fab, scFv, or immunoglobulin) is immobilized. In some embodiments, the immunoaffinity reagent, such as an antigen-binding molecule is fused or linked to a binding partner that interacts with a binding reagent immobilized on the matrix. In some embodiments, the binding capacity of the chromatography matrix is sufficient to adsorb or is capable of adsorbing at least 1×10⁷ cells/mL, 5×10⁷ cells/mL, 1×10⁸ cells/mL, 5×10⁸ cells/mL, 1×10⁹ cells/mL or more, in which said cells are cells expressing a cell surface marker specifically recognized by the affinity reagent, such as antibody or Fab.

In some embodiments, the interaction between the binding reagent and binding partner forms a reversible bond, so that binding of the antigen-binding molecule to the matrix is reversible. In some embodiments, the reversible binding can be mediated by a streptavidin mutant binding partner and a binding reagent immobilized on the matrix that is streptavidin, a streptavidin analog or mutein, avidin or an avidin analog or mutein.

In some embodiments, reversible binding of the affinity reagent, such as antigen-binding molecule (e.g., an Fab, scFv, or immunoglobulin) is via a peptide ligand binding reagent and streptavidin mutein interaction, as described above with respect to immunoaffinity beads. In aspects of the chromatography matrix, the matrix, such as agarose beads or other matrix, is functionalized or conjugated with a streptavidin mutein, such as any described above. In some embodiments, the antigen-binding molecule (e.g., an Fab, scFv, or immunoglobulin) is fused or linked, directly or indirectly, to a peptide ligand capable of binding to a streptavidin mutant, such as any described above. In some embodiments, the chromatography matrix column is contacted with such an affinity reagent, such as an antigen-binding molecule (e.g., an Fab, scFv, or immunoglobulin) to immobilize or reversibly bind the affinity reagent to the column.

In some embodiments, the immunoaffinity chromatography matrix can be used in enrichment and selection methods as described herein by contacting the matrix with a sample containing cells to be enriched or selected. In some embodiments, the selected cells are eluted or released from the matrix by disrupting the interaction of the binding partner/binding reagent. In some embodiments, binding partner/binding reagents is mediated by a peptide ligand and streptavidin mutant interaction, and the release or selected cells can be effected due to the presence of a reversible bond. For example, in some embodiments, the bond between the peptide ligand binding partner and streptavidin mutein binding reagent is high, such as described above, but is less than the binding affinity of the streptavidin binding reagent for biotin or a biotin analog. Hence, in some embodiments, biotin (Vitamin H) or a biotin analog can be added to compete for binding to disrupt the binding interaction between the streptavidin mutein binding reagent on the matrix and the peptide ligand binding partner associated with the antibody specifically bound to a cell marker on the surface. In some embodiments, the interaction can be reversed in the presence of low concentrations of biotin or analog, such as in the presence of 0.1 mM to 10 mM, 0.5 mM to 5 mM or 1 mM to 3 mM, such as generally at least or about at least 1 mM or at least 2 mM, for example at or about 2.5 mM. In some embodiments, elution in the presence of a competing agent, such as a biotin or biotin analog, releases the selected cell from the matrix.

In some embodiments, immunoaffinity chromatography in the disclosed herein manufacturing processes is performed using a chromatography matrix column, whereby an affinity or binding agent to CD49f, such as antigen-binding molecule (e.g., an Fab, scFv, or immunoglobulin) that specifically binds to CD49f is coupled to a first chromatography matrix in a first selection column.

A T-cell-containing sample is loaded onto to the column and a wash buffer is typically used to wash out non-bound cells from the columns. CD49f⁺ cells are subsequently eluted from the column using an elution buffer. The washing buffer can be any physiological buffer that is compatible with cells, such as phosphate buffered saline. In some embodiments, the washing buffer contains bovine serum albumin, human serum albumin, or recombinant human serum albumin, such as at a concentration of 0.1% to 5% or 0.2% to 1%, such as or at about 0.5%. In some embodiments, the eluent is biotin or a biotin analog, such as desbiotin, for example in an amount that is or is about at least 0.5 mM, 1 mM, 1.5 mM, 2 mM, 2.5 mM, 3 mM, 4 mM, or 5 mM.

In some embodiments, at least one additional affinity reagent specifically binds a marker on T-cells (e.g., CD4 and/or CD8), and optionally on naïve, resting or central memory T-cells or specifically binds a marker selected from CD95, CD45RO, CD62L, CCR7, CD28, CD3, CD27 and CD127.

3.5 Enrichments and Ratios of Generated Compositions

In some embodiments, the manufacturing processes produce an enriched composition of cells containing a population of enriched cells, such as a population of cells enriched for CD49f⁺ cells, and optionally enriched for one or both of CD4 and CD8 and optionally markers on naïve, resting or central memory T-cells or (e.g., selected from one or more of CD95, CD45RO, CD62L, CCR7, CD28, CD3, CD27 and CD127). In some embodiments, the enriched composition of cells is designated a culture initiation composition and is used in subsequent processing steps, such as subsequent processing steps involving incubation, stimulation, activation, engineering and/or formulation of the enriched cells. In some embodiments, subsequent to the further processing steps, such as processing steps involving incubation, stimulation, activation, engineering and/or formulation, and output composition is generated that, in some aspects, can contain genetically engineered cells containing CD49f⁺ cells expressing a genetically engineered antigen receptor (e.g., a rTCR or a CAR).

In some embodiments, the enriched compositions of cells are enriched cells from a starting sample as describe above, in which the number of cells in the starting sample is at least greater than the desired number of cells in an enriched composition, such as a culture-initiation composition. In some embodiments, the number of cells in the starting sample is greater by at least 10%, 20%, 30%, 40%, 50%, 60%, 70%, 80%, 90%, 100%, 500%, 1000%, 5000% or more greater than the desired number of cells in the enriched composition. In some examples, the desired number of cells in the enriched population, including enriched CD49f⁺ cells or sub-populations thereof, is at least 1×10⁶ cells, 2×10⁶ cells, 4×10⁶ cells 6×10⁶ cells 8×10⁶ cells, 1×10⁷ cells, 2×10⁷ cells, 4×10⁷ cells, 6×10⁷ cells, 8×10⁷ cell, 1×10⁸ cells, 2×10⁸ cells, 4×10⁸ cells, 6×10⁸ cells, 8×10⁸ cells, 1×10⁹ cells or greater. In some embodiments, the number of cells in the starting sample, is at least 1×10⁸ cells, 5×10⁸ cells, 1×10⁹ cells, 2×10⁹ cells, 3×10⁹ cells, 4×10⁹ cells, 5×10⁹ cells, 6×10⁹ cells, 7×10⁹ cells, 8×10⁹ cells, 9×10⁹ cells, 1×10¹⁰ cells or more.

In some embodiments, the yield of the population or sub-population thereof, in the enriched composition, i.e., the number of enriched cells in the population or sub-population compared to the number of the same population or sub-population of cells in the starting sample, is 10% to 100%, such as 20% to 80%, 20% to 60%, 20% to 40%, 40% to 80%, 40% to 60%, or 60%, to 80%. In some embodiments, the yield of the population of cells or sub-population thereof is less than 70%, less than 60%, less than 50%, less than 40%, less than 30% or less than 20%.

In some embodiments, the purity of the population of cells or sub-population of cells thereof in the enriched composition, i.e., the percentage of cells positive for the selected cell surface marker (e.g., CD49f) versus total cells in the population of enriched cells, is at least 60%, 65%, 70%, 75%, 80%, 85%, 90%, 91%, 92%, 93%, 94%, and is generally at least 95%, 96%, 97%, 98%, 99% or greater.

3.6 Incubation of Isolated Cells

In some embodiments, the manufacturing processes include one or more of various steps for incubating isolated cells and cell populations, such as populations isolated according to the manufacturing processes disclosed herein, such as steps for incubating an isolated CD49f⁺ T-cell population. The isolated cell population (e.g., unfractionated or subpopulations thereof) is generally incubated in a culture-initiating composition in a culture vessel, such as a chamber, well, column, tube, tubing set, valve, vial, culture dish, bag, or other container for culture or cultivating cells.

The incubation steps can include culture, cultivation, stimulation, activation, propagation, including by incubation in the presence of stimulating conditions, for example, conditions designed to induce proliferation, expansion, activation, and/or survival of cells in the population, to mimic antigen exposure, and/or to prime the cells for genetic engineering, such as for the introduction of a genetically engineered antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In one example, the stimulating conditions include one or more agent, e.g., ligand, which turns on or initiates TCR/CD3 intracellular signaling cascade in a T-cell. Such agents can include antibodies, such as those specific for a TCR component and/or co-stimulatory receptor, e.g., anti-CD3, anti-CD28, anti-4-1BB, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). Optionally, the expansion method may further comprise the step of adding IL-2 and/or IL-15 and/or IL-7 and/or IL-21 to the culture medium (e.g., wherein the concentration of IL-2 is at least about 10 units/mL).

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the cell populations, such as CD49f⁺ populations or subpopulations, are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T-cells). In some embodiments, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T-cells.

In some embodiments, the stimulating conditions include temperature suitable for the growth of human T lymphocytes, for example, at least about 25° C., generally at least about 30° C., and generally at or about 37° C. In some embodiments, a temperature shift is effected during culture, such as from 37° C. to 35° C. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, populations or subpopulations of CD49f⁺ that are antigen-specific can be obtained by stimulating naïve or antigen-specific T lymphocytes with antigen. For example, antigen-specific T-cell lines or clones can be generated to a cancer or tumor-associated antigen, an infectious disease-associated antigen, an autoimmune disease-associated antigen, a transplantation antigen or an allergen by isolating T-cells from affected subjects and stimulating the cells in vitro with the same antigen.

3.7 Interim Assessment and Adjustment

In some embodiments, the manufacturing processes include assessment and/or adjustment of the cells or composition containing the cells, at a time subsequent to the initiation of the incubation or culture, such as at a time during the incubation. Assessment can include taking one or more measurements of a composition or vessel containing the cells, such as assessing cells for proliferation rate, degree of survival, phenotype, e.g., expression of one or more surface or intracellular markers, such as proteins or polynucleotides, and/or assessing the composition or vessel for temperature, media component(s), oxygen or carbon dioxide content, and/or presence or absence or amount or relative amount of one or more factors, agents, components, and/or cell types, including subtypes. Assessment in some embodiments includes determining an intermediate ratio of a plurality, e.g., two cell types, such as CD49f⁺ CD4⁺ and CD49f⁺ CD8⁺ T-cells, including CD49f⁺ CD4⁺ and CD49f⁺ CD8⁺ central memory T-cells, in the composition or vessel being incubated. In some aspects, the assessment is performed in an automated fashion, for example, using a device as described herein, and/or is set ahead of time to be carried out at certain time-points during incubation. In some embodiments, the outcome of the assessment, such as a determined interim ratio of two types of cells (e.g., CD49f⁺ CD4⁺ and CD49f⁺ CD8⁺ T-cells), indicates that an adjustment should be made, such as addition or removal of one or more cell types.

In some embodiments, where cells are engineered, e.g., to introduce a genetically engineered antigen receptor, the incubation in the presence of one or more stimulating agents continues during the engineering phase.

In some embodiments, the cells are incubated for at or about 1, 2, 3, 4, 5, 6, 7, 8, 9, 10, 11, 12, 13, 14, 15, 16 or 17 days, either in total or prior to engineering.

3.8 Engineering, Engineered Antigen Receptors, and Engineered Cells

In some embodiments, the manufacturing processes include genetic engineering of the isolated and/or incubated cells, such as to introduce into the cells recombinant genes for expression of molecules, such as receptors, e.g., antigen receptors, useful in the context of adoptive therapy.

Among the genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. This can be carried out in accordance with known techniques (see, e.g., Riddell et al., U.S. Pat. No. 6,040,177, at columns 14-17) or variations thereof that will be apparent to those skilled in the art based upon the present disclosure.

The engineering generally includes introduction of gene or genes for expression of a genetically engineered antigen receptor. Among such antigen receptors are genetically engineered or recombinant T-cell receptors (rTCRs) and components thereof, and functional non-TCR antigen receptors, such as chimeric antigen receptors (CAR).

The antigen receptor in some embodiments specifically binds to a ligand on a cell or disease to be targeted, such as a cancer or other disease or condition, including those described herein for targeting with the disclosed herein methods and compositions. Exemplary antigens are orphan tyrosine kinase receptor ROR1, tEGFR, Her2, Li-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acetylcholine E receptor, GD2, GD3, HMW-MAA, IL-22R-alpha, IL-13R-alpha2, kdr, kappa light chain, Lewis Y, Li-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3 and/or biotinylated molecules, and/or molecules expressed by pathogens such as Epstein-Barr virus (EBV), cytomegalovirus (CMV), human immunodeficiency virus (HIV), hepatitis C virus (HCV), hepatitis B virus (HBV) or other pathogens.

3.8.1 Antigen Receptors

In some embodiments, the engineered antigen receptors are CARs. The CARs generally include genetically engineered receptors including an extracellular ligand binding domain linked to one or more intracellular signaling components. Such molecules typically mimic or approximate a signal through a natural antigen receptor and/or signal through such a receptor in combination with a costimulatory receptor.

In some embodiments, CARs are constructed with specificity for a particular marker, such as a marker expressed in a particular cell type to be targeted by adoptive therapy, e.g., a cancer marker. This is achieved in some aspects by inclusion in the extracellular portion of the CAR one or more antigen binding molecule, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the CAR comprises an antibody heavy chain domain that specifically binds a cell surface antigen of a cell or disease to be targeted, such as a tumor cell or a cancer cell, such as any of the target antigens described herein or known in the art.

In some embodiments, the tumor antigen or cell surface molecule is a polypeptide. In some embodiments, the tumor antigen or cell surface molecule is selectively expressed or overexpressed on tumor cells as compared to non-tumor cells of the same tissue.

In some embodiments, the CAR binds a pathogen-specific antigen. In some embodiments, the CAR is are specific for viral antigens (such as EBV, CMV, HIV, HCV, HBV, etc.), bacterial antigens, and/or parasitic antigens.

In some preferred embodiments, the CAR targets CD19. In some other embodiments, the CAR targets any one of the group comprising: CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signalling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumour-associated glycoprotein (TAG)-72, CD38, CD44v6, carcinoembryonic antigen (CEA), epithelial cell adhesion molecule (EpCAM), KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGS5), high molecular weight melanoma-associated antigen (HMWMAA), o-acetyl-GD2 ganglioside, folate receptor beta, TEM1/CD248, tumour endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor .gamma.-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYPiB1), CCCTC-Binding Factor (Zinc Finger Protein)-Like (BORIS or Brother of the Regulator of Imprinted Sites), squamous Cell Carcinoma Antigen Recognized By T Cells 3 (SART3), PAX5, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), signalling lymphocyte activation molecule (SLAM) family member 6 (SLAMF6), SLAMF4, or any combination thereof.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the α, β or ζ chain of the T-cell receptor, CD28, CD3-E, CD45, CD4, CD5, CD8, CD9, CD16, CD22, CD33, CD37, CD64, CD80, CD86, CD 134, CD137, CD154. Alternatively the transmembrane domain in some embodiments is synthetic. Suitably, the synthetic transmembrane domain comprises predominantly hydrophobic residues such as leucine and valine. In some aspects, a triplet of phenylalanine, tryptophan and valine will be found at each end of a synthetic transmembrane domain.

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The CAR generally includes intracellular signaling component or components. In some embodiments, the CAR includes an intracellular component of the TCR complex, such as a TCR CD3⁺ chain that mediates T-cell activation and cytotoxicity, e.g., CD3-ζ chain. Thus, in some embodiments, the antigen binding molecule is linked to one or more cell signaling modules. In some embodiments, cell signaling modules include CD3 transmembrane domain, CD3 intracellular signaling domains, and/or other CD transmembrane domains. In some embodiments, the CAR further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor .gamma. and CD8, CD4, CD25 or CD16.

In some embodiments, upon ligation of the CAR, the cytoplasmic domain or intracellular signaling domain of the CAR activates at least one of the normal effector functions of the immune cell, e.g., T-cell engineered to express the cell. For example, in some contexts, the CAR induces a function of a T-cell such as cytolytic activity or T-helper activity, such as secretion of cytokines or other factors. In some embodiments, a truncated portion of an intracellular signaling domain of an antigen receptor component or costimulatory molecule. Such truncated portion in some aspects is used in place of an intact immunostimulatory chain, for example, if it transduces the effector function signal. In some embodiments, the intracellular signaling domain or domains include the cytoplasmic sequences of the T-cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. T-cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

Primary cytoplasmic signaling sequences can in some aspects regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR-ζ, FcR-γ, FcR-β, CD3-γ, CD3-δ, CD3-ε, CDS, CD22, CD79a, CD79b, and CD66d. In some embodiments, cytoplasmic signaling molecule(s) in the CAR contain(s) a cytoplasmic signaling domain, portion thereof, or sequence derived from CD3-ζ.

In some embodiments, the CAR includes a signaling domain and/or transmembrane portion of a costimulatory receptor, such as CD28, 4-1BB, OX40, DAP10, and ICOS.

In certain embodiments, the intracellular signaling domain comprises a CD28 transmembrane and signaling domain linked to a CD3 intracellular domain. In some embodiments, the intracellular signaling domain comprises a chimeric CD28 and CD137 co-stimulatory domains, linked to a CD3 intracellular domain. In some embodiments, a CAR can also include a transduction marker (e.g., tEGFR). In some embodiments, the intracellular signaling domain of the CD8⁺ cytotoxic T-cells is the same as the intracellular signaling domain of the CD4⁺ helper T-cells. In some embodiments, the intracellular signaling domain of the CD8⁺ cytotoxic T-cells is different than the intracellular signaling domain of the CD4⁺ helper T-cells.

In some embodiments, the CAR encompasses two or more costimulatory domain combined with an activation domain, e.g., primary activation domain, in the cytoplasmic portion. One example is a receptor including intracellular components of CD3-ζ, CD28, and 4-1BB.

CARs and production and introduction thereof can include those described, for example, by published patent disclosures WO200014257, U.S. Pat. No. 6,451,995, US2002131960, U.S. Pat. Nos. 7,446,190, 8,252,592, EP2537416, US2013287748, and WO2013126726, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75.

Representative CAR T-cells contemplated by the present disclosure include TRUCKs, Universal CARs, Self-driving CARs, Armored CARs, Self-destruct CARs, Conditional CARs, Marked CARs, TenCARs, Dual CARs, and safety CARs.

For example, TRUCKs co-express a chimeric antigen receptor (CAR) and an immune-stimulatory cytokine (e.g., IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β). Cytokine expression may be constitutive or induced by T-cell activation. Targeted by CAR specificity, localized production of pro-inflammatory cytokines recruits endogenous immune cells to tumor sites and may potentiate an antitumor response.

Universal, allogeneic CAR T-cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T-cells engineered to be resistant to immunosuppression (Armored CARs) may be genetically modified to no longer express various immune checkpoint molecules (for example, cytotoxic T lymphocyte-associated antigen 4 (CTLA-4) or programmed cell death protein 1 (PD-1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T-cell may be achieved based on ganciclovir binding to thymidine kinase in gene-modified lymphocytes or the more recently described system of activation of human caspase 9 by a small-molecule dimerizer.

A conditional CAR T-cell is by default unresponsive, or switched ‘off’, until the addition of a small molecule to complete the circuit, enabling full transduction of both signal 1 and signal 2, thereby activating the CAR T cell. Alternatively, T-cells may be engineered to express an adaptor-specific receptor with affinity for subsequently administered secondary antibodies directed at target antigen.

Marked CAR T-cells express a CAR plus a tumor epitope to which an antigen-binding molecule binds. In the setting of intolerable adverse effects, administration of the antigen-binding molecule (e.g., monoclonal antibody) clears the CAR T-cells and alleviates symptoms with no additional off-tumor effects.

A tandem CAR (TanCAR) T-cell expresses a single CAR consisting of two linked single-chain variable fragments (scFvs) that have different affinities fused to intracellular co-stimulatory domain(s) and a CD3-ζ domain. TanCAR T cell activation is achieved only when target cells co-express both targets.

A dual CAR T-cell expresses two separate CARs with different ligand binding targets; one CAR includes only the CD3-ζ domain and the other CAR includes only the co-stimulatory domain(s). Dual CAR T-cell activation requires co-expression of both targets on the tumor.

A safety CAR (sCAR) consists of an extracellular scFv fused to an intracellular inhibitory domain, sCAR T-cells co-expressing a standard CAR become activated only when encountering target cells that possess the standard CAR target but lack the sCAR target.

In some embodiments, the T-cells are modified with a recombinant T-cell receptor (rTCR). In some embodiments, the rTCR is specific for an antigen, generally an antigen present on a target cell, such as a tumor-specific antigen, an antigen expressed on a particular cell type associated with an autoimmune or inflammatory disease, or an antigen derived from a pathogen (e.g., a viral pathogen or a bacterial pathogen).

In some embodiments, the T-cells are engineered to express T-cell receptors (TCRs) cloned from naturally occurring T-cells. In some embodiments, a high-affinity T-cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, after the T-cell clone is obtained, the TCR α and β chains are isolated and cloned into a gene expression vector. In some embodiments, the TCR α and β genes are linked via a picornavirus 2A ribosomal skip peptide so that both chains are co-expressed. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; an Hackett et al. (2010) Molecular Therapy: The Journal of the American Society of Gene Therapy. 18:674-683.

In some embodiments, gene transfer is accomplished by first stimulating T-cell growth and the activated cells are then transduced and expanded in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines, such as proinflammatory cytokines, e.g., IL-2, IL-12, IL-7, IL-15, IL-21.

3.8.2 Introduction of the Genetically Engineered Components

Various methods for the introduction of genetically engineered components, e.g., antigen receptors, e.g., rTCRs, CARs, are well known and may be used with the disclosed herein methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T-cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November; 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T-cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T-cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, a CAR is introduced into the CD49f⁺ T-cell populations or subpopulations. In some embodiments, different CARs are introduced into the CD49f⁺ T-cell populations or subpopulations. Suitably, the different CARs each have an antigen binding molecule that specifically binds to the same antigen, or to different antigens. In some embodiments, the different CARs have cellular signaling modules that differ. In some embodiments, CD49f⁺ T-cell populations or subpopulations have been sorted in to naïve, central memory, effector memory or effector cells prior to transduction.

In other embodiments, the cells, e.g., T-cells, are not engineered to express recombinant receptors, but rather include naturally occurring antigen receptors specific for desired antigens, such as tumor-infiltrating lymphocytes and/or T-cells cultured in vitro or ex vivo, e.g., during the incubation step(s), to promote expansion of cells having particular antigen specificity. For example, in some embodiments, the cells are produced for adoptive cell therapy by isolation of tumor-specific T-cells, e.g. autologous tumor infiltrating lymphocytes (TIL). The direct targeting of human tumors using autologous tumor infiltrating lymphocytes can in some cases mediate tumor regression (see Rosenberg S A, et al. (1988) N Engl J Med. 319:1676-1680). In some embodiments, lymphocytes are extracted from resected tumors. In some embodiments, such lymphocytes are expanded in vitro. In some embodiments, such lymphocytes are cultured with lymphokines (e.g., IL-2). In some embodiments, such lymphocytes mediate specific lysis of autologous tumor cells but not allogeneic tumor or autologous normal cells.

3.9 D. Cryopreservation

In some embodiments, the disclosed herein manufacturing processes include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to 80.degree. C. at a rate of 1.degree. per minute and stored in the vapor phase of a liquid nitrogen storage tank.

### 4. Kits for the Disclosed Manufacturing Processes

Also disclosed herein are kits useful for carrying out the manufacturing processes disclosed herein. In some embodiments, the kits include antigen-binding molecules or other binding partners, generally coupled to solid supports, for the isolation, e.g., for immunoaffinity-based separation steps, of the manufacturing processes.

In some embodiments, the kit comprises antigen-binding molecules for positive and negative selection, bound to magnetic beads. In one embodiment, the kit comprises instructions to carry out selection starting with a sample, such as a PBMC sample, by selecting based on expression of a first surface marker, recognized by one or more of the antigen-binding molecules provided with the kit, retaining both positive and negative fractions. In some aspects, the instructions further include instructions to carry out one or more additional selection steps, starting with the positive and/or negative fractions derived therefrom, for example, while maintaining the compositions in a contained environment and/or in the same separation vessel.

In some embodiments, the kit comprises an anti-CD49f antigen-binding molecule, and optionally one or more of anti-CD4, anti-CD8, anti-CD95, anti-CD27, anti-CD28, anti-CCR7, anti-CD14, anti-CD45RA, anti-CD14, and anti-CD62L antigen-binding molecules, bound to magnetic beads. In some embodiments, the kit comprises instructions to carry out selection starting with a sample, such as a PBMC sample, by selecting based on CD49f expression, retaining both positive and negative fractions, and on the negative fraction, further subjecting the fraction to a negative selection using for example the anti-CD14, anti-CD45RA antibodies, and a positive selection using the anti-CD62L antibody, in either order. Alternatively, the components and instructions are adjusted according to any of the separation embodiments described herein.

In some embodiments, the kit further includes instructions to transfer the cells of the populations isolated by the selection steps to a culture, cultivation, or processing vessel, while maintaining the cells in a self-contained system. In some embodiments, the kit includes instructions to transfer the different isolated cells at a particular ratio.

### 5. Cells, Compositions, and Methods of Administration

Also disclosed herein are cells, cell populations, and compositions (including pharmaceutical and therapeutic compositions) containing the cells and populations, produced by the manufacturing processes disclosed herein. Also disclosed herein are methods, e.g., therapeutic methods for administrating the cells and compositions to subjects, e.g., patients.

In particular, disclosed herein are methods of administering the cells, populations, and compositions, and uses of such cells, populations, and compositions to treat or prevent diseases, conditions, and disorders, including cancers. In some embodiments, the cells, populations, and compositions are administered to a subject or patient having the particular disease or condition to be treated, e.g., via adoptive cell therapy, such as adoptive T-cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T-cell.

Methods for administration of cells for adoptive cell therapy are known and may be used in connection with the provided methods and compositions. For example, adoptive T-cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T-cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T-cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

Also disclosed herein are pharmaceutical compositions for use in such methods.

Among the diseases, conditions, and disorders for treatment with the provided compositions, cells, methods and uses are tumors, including solid tumors, hematologic malignancies, and melanomas, and infectious diseases, such as infection with a virus or other pathogen, e.g., EBV, CMV, HIV, HCV, HBV, and parasitic disease. In some embodiments, the disease or condition is a tumor, cancer, malignancy, neoplasm, or other proliferative disease. Such diseases include but are not limited to leukemia, lymphoma, e.g., chronic lymphocytic leukemia (CLL), ALL, non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin (including melanoma), bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

In some embodiments, the disease or condition is an infectious disease or condition, such as, but not limited to, viral, retroviral, bacterial, and protozoal infections, immunodeficiency, CMV, EBV, adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant.

In some embodiments, the antigen associated with the disease or disorder is selected from the group consisting of orphan tyrosine kinase receptor ROR1, tEGFR, Her2, Li-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, 0EPHa2, ErbB2, 3, or 4, FBP, fetal acetylcholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3 and/or biotinylated molecules, and/or molecules expressed by CMV, EBV, HIV, HCV, HBV or other pathogens.

In some embodiments, the cells and compositions are administered to a subject in the form of a pharmaceutical composition, such as a composition comprising the cells or cell populations and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulfonic acids, for example, p-toluenesulfonic acid.

The choice of carrier in the pharmaceutical composition is determined in part by the particular engineered CAR or TCR, vector, or cells expressing the CAR or TCR, as well as by the particular method used to administer the vector or host cells expressing the CAR. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

In some embodiments, the pharmaceutical composition employs time-released, delayed release, and/or sustained release delivery systems, such that the delivery of the composition occurs prior to, and with sufficient time to cause, sensitization of the site to be treated. Many types of release delivery systems are available and known to those of ordinary skill in the art. Such systems in some aspects can avoid repeated administrations of the composition, thereby increasing convenience to the subject and the physician.

In some embodiments, the pharmaceutical composition comprises the cells or cell populations in an amount that is effective to treat or inhibit the development of the disease or condition, such as a therapeutically effective or prophylactically effective amount. Thus, in some embodiments, the methods of administration include administration of the cells and populations at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the cells are administered at a desired dosage, which in some aspects includes a desired dose or number of cells or cell type(s) and/or a desired ratio of cell types. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations such as CD49f⁺ T-cells, or sub-types of cells such as CD49f⁺ CD8⁺ T-cells and CD49f⁺ CD4⁺ T-cells, are administered at or within a tolerated difference of a desired dose of total cells, such as a desired dose of T-cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD49f⁺ CD4⁺ to CD49f⁺ CD8⁺ ratio), e.g., within a certain tolerated difference or error of such a ratio.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 104 and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 104 and at or about 109 T-cells/kilograms (kg) body weight, such as between 10⁵ and 10⁶ T-cells/kg body weight, for example, at or about 1×10⁵ T-cells/kg, 1.5×10⁵ T-cells/kg, 2×10⁵ T-cells/kg, or 1×10⁶ T-cells/kg body weight.

In some embodiments in which different subtypes of CD49f⁺ T-cells are used, such as CD49f⁺ CD8⁺ and CD49f⁺ CD4⁺ T-cells subsets, the cells may be administered at or within a certain range of error of between at or about 104 and at or about 109 CD49f⁺ CD4⁺ and/or CD49f⁺ CD8⁺ cells/kilograms (kg) body weight, such as between 10⁵ and 10⁶ CD49f⁺ CD4⁺ and/or CD49f⁺ CD8⁺ cells/kg body weight, for example, at or about 1×10⁵ CD49f⁺ CD4⁺ and/or CD49f⁺ CD8⁺ cells/kg, 1.5×10⁵ CD49f⁺ CD4⁺ and/or CD49f⁺ CD8⁺ cells/kg, 2×10⁵ CD49f⁺ CD4⁺ and/or CD49f*CD8⁺ cells/kg, or 1×10⁶ CD49f⁺ CD4⁺ and/or CD49f⁺ CD8⁺ cells/kg body weight.

In some embodiments in which different subtypes of CD49f⁺ T-cells are used, such as CD49f⁺ CD8⁺ and CD49f⁺ CD4⁺ T-cells subsets, the cells are administered at or within a tolerated range of a desired output ratio of multiple cell populations or sub-types, such as CD49f⁺ CD4⁺ and CD49f⁺ CD8⁺ cells or sub-types. In some aspects, the desired ratio can be a specific ratio or can be a range of ratios. for example, in some embodiments, the desired ratio (e.g., ratio of CD49f⁺ CD4⁺ to CD49f⁺ CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

The cell populations and compositions in some embodiments are administered to a subject using standard administration techniques, including oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

The cell populations obtained using the methods described herein in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

Following administration of the cells, the biological activity of the engineered cell populations in some embodiments is measured, e.g., by any of a number of known methods. Parameters to assess include specific binding of an engineered or natural T-cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 111 (1995), and U.S. Pat. No. 5,087,616.

### 6. Articles of Manufacture

Also provided are articles of manufacture, such as kits and devices, for the administration of the cells to subjects in according to the provided methods for adoptive cell therapy, and for storage and administration of the cells and compositions.

The articles of manufacture include one or more containers, typically a plurality of containers, packaging material, and a label or package insert on or associated with the container or containers and/or packaging, generally including instructions for administration of the cells to a subject.

The containers generally contain the cells to be administered, e.g., one or more unit doses thereof. The article of manufacture typically includes a plurality of containers, each containing a single unit dose of the cells. The unit dose may be an amount or number of the cells to be administered to the subject in the first dose or twice the number (or more) the cells to be administered in the first or consecutive dose(s). It may be the lowest dose or lowest possible dose of the cells that would be administered to the subject in connection with the administration method. In some embodiments, the unit dose is the minimum number of cells or number of cells that would be administered in a single dose to any subject having a particular disease or condition or any subject, according to the methods herein. For example, the unit dose in some aspects may include a minimum number of cells that would be administered to a patient of a relatively lower body weight and/or with relatively low disease burden, such that one and in some cases more than one unit dose is administered to a given subject as a first dose and one or more than one unit dose is administered to a given subject in one or more consecutive dose, e.g., according to the provided methods. In some embodiments, the number of cells in the unit dose is the number of CD49f⁺ T-cells, and/or the number of CD49f⁺ T-cell sub-types such as CD49f⁺ CD8⁺ T-cells and CD49f⁺ CD4⁺ T-cells, that it is desired to administer to a particular subject in a first dose, such as a subject from which the cells have been derived. In some embodiments, the cells have been derived from the subject to be treated by methods as provided herein or in need thereof. In some of the same and other embodiments, the number of cells in the unit dose is the number of cells or number of recombinant receptor-expressing or CAR-expressing cells that it is desired to administer to a particular subject in a first dose, such as a subject from which the cells have been derived. In some embodiments, the cells have been derived from the subject to be treated by methods as provided herein or in need thereof.

In some embodiments, each of the containers individually comprises a unit dose of the cells, e.g., including the same or substantially the same number of cells. Thus in some embodiments, each of the containers comprises the same or approximately or substantially the same number of cells or number of recombinant receptor-expressing cells. In some embodiments, the unit dose includes less than about 1×10⁸, less than about 5×10⁷, less than about 1×10⁶ or less than about 5×10⁵ of the CD49f⁺ T-cells, of engineered cells, of total cells, or PBMCs, per kg of the subject to be treated and/or from which the cells have been derived. In some embodiments, each unit dose contains at or about 2×10⁶, 5×10⁶, 1×10⁷, 5×10⁷, or 1×10⁸ CD49f⁺ T-cells, engineered cells, total cells, or PBMCs.

Suitable containers include, for example, bottles, vials, syringes, and flexible bags, such as infusion bags. In particular embodiments, the containers are bags, e.g., flexible bags, such as those suitable for infusion of cells to subjects, e.g., flexible plastic or PVC bags, and/or IV solution bags. The bags in some embodiments are sealable and/or able to be sterilized, so as to provide sterile solution and delivery of the cells and compositions. In some embodiments, the containers, e.g., bags, have a capacity of at or about or at least at or about 10, 20, 30, 40, 50, 60, 70, 80, 90, 100, 200, 300, 400, 500, or 1000 ml capacity, such as between at or about 10 and at or about 100 or between at or about 10 and at or about 500 mL capacity. In some embodiments, the containers, e.g., bags, are and/or are made from material which is stable and/or provide stable storage and/or maintenance of cells at one or more of various temperatures, such as in cold temperatures, e.g. below at or about or at or about −20° C., −80° C., −120° C., 135° C. and/or temperatures suitable for cryopreservation, and/or other temperatures, such as temperatures suitable for thawing the cells and body temperature such as at or about 37° C., for example, to permit thawing, e.g., at the subject's location or location of treatment, e.g., at bedside, immediately prior to treatment.

The containers may be formed from a variety of materials such as glass or plastic. In some embodiments, the container has one or more port, e.g., sterile access ports, for example, for connection of tubing or cannulation to one or more tubes, e.g., for intravenous or other infusion and/or for connection for purposes of transfer to and from other containers, such as cell culture and/or storage bags or other containers. Exemplary containers include infusion bags, intravenous solution bags, vials, including those with stoppers pierceable by a needle for injection.

The article of manufacture may further include a package insert or label with one or more pieces of identifying information and/or instructions for use. In some embodiments, the information or instructions indicates that the contents can or should be used to treat a particular condition or disease, and/or providing instructions therefor. The label or package insert may indicate that the contents of the article of manufacture are to be used for treating the disease or condition. In some embodiments, the label or package insert provides instructions to treat a subject, e.g., the subject from which the cells have been derived, via a method involving the administration of a first and one or more consecutive doses of the cells, e.g., according to any of the embodiments of the provided methods. In some embodiments, the instructions specify administration, in a first dose, of one unit dose, e.g., the contents of a single individual container in the article of manufacture, followed by one or more consecutive doses at a specified time point or within a specified time window and/or after the detection of the presence or absence or amount or degree of one or more factors or outcomes in the subject.

In some embodiments, the instructions specify administering a plurality of the unit doses to the subject by carrying out a first administration and a consecutive administration. In some embodiments, the first administration comprises delivering one of said unit doses to the subject and the consecutive administration comprises administering one or a plurality of said unit doses to the subject.

In some embodiments, the instructions specify that the consecutive administration is to be carried out at a time between about 15 and about 27 days or between about 9 and about 35 days, e.g., at or about 21 days, following the first administration, e.g., following the initiation of the first administration or the prior administration. In some embodiments, the instructions specify that the consecutive dose is to be administered at a time after which it has been determined that a serum level of a factor indicative of cytokine-release syndrome (CRS) in the subject is less than about 10 times, less than about 25 times, and/or less than about 50 times the serum level of the indicator in the subject immediately prior to said first administration, and/or that an indicator of CRS has peaked and is declining, and/or that the subject does not exhibit a detectable adaptive host immune response specific for a disease associated antigen or a receptor, e.g., a natural TCR, rTCR or CAR, expressed by the cells.

In some embodiments, the label or package insert or packaging comprises an identifier to indicate the specific identity of the subject from which the cells are derived and/or are to be administered. In the case of autologous transfer, the identity of the subject from which the cells are derived is the same as the identity of the subject to which the cells are to be administered. Thus, the identifying information may specify that the cells are to be administered to a particular patient, such as the one from which the cells were originally derived. Such information may be present in the packaging material and/or label in the form of a bar code or other coded identifier, or may indication the name and/or other identifying characteristics of the subject.

The article of manufacture in some embodiments includes one or more, typically a plurality, of containers containing compositions comprising the cells, e.g., individual unit dose forms thereof, and further include one or more additional containers with a composition contained therein which includes a further agent, such as a cytotoxic or otherwise therapeutic agent, for example, which is to be administered in combination, e.g., simultaneously or sequentially in any order, with the cells. Alternatively, or additionally, the article of manufacture may further include another or the same container comprising a pharmaceutically-acceptable buffer. It may further include other materials such as other buffers, diluents, filters, tubing, needles, and/or syringes.

### 7. Assessing Competence of T-Cell Populations for Immunotherapy

In accordance with the present disclosure, competence of a T-cell population for immunotherapy is assessed by determining a level or concentration of CD49f⁺ T-cells in the T-cell population. The T-cell population can be any T-cell-containing sample, including primary cell sample such as a primary human cell sample, as described for example above and cultured cells including T-cell lines.

In some embodiments, the level or concentration of CD49f⁺ T-cells comprises a level or concentration of CD49f^{hi} T-cells only, a level or concentration of CD49f^{int} T-cells only, or a level or concentration of both CD49f^{hi} T-cells and CD49f^{int} T-cells. In some embodiments, the CD49f⁺ T-cells comprise memory T-cells (e.g., central memory T-cells), such as, but not limited to, the following memory T-cell subtypes: CD49f⁺ CD27⁺ CD28⁺ memory T-cells; CD49f⁺ CD27⁺ CD28⁺ CD45RA⁺ memory T-cells; CD49f⁺ CD27⁺ CD28⁺ CCR7⁺; memory T-cells and CD49f⁺ CD27⁺ CD28⁺ CD45RA⁺ CCR7⁺ memory T-cells. In some of the same and other embodiments, the CD49f⁺ T-cells comprise CD8⁺ CD49f⁺ T-cells, CD4⁺ CD49f⁺ T-cells or both CD8⁺ CD49f⁺ T-cells and CD4⁺ CD49f⁺ T-cells. In some of the same and other embodiments, the CD49f⁺ T-cells comprise T-cells that have an early memory phenotype and/or a stem-like phenotype. In illustrative examples of this type, the CD49f⁺ T-cells are positive for TCF-1 (e.g., TCF-1^{hi}) and/or LEF-1 (e.g., LEF-1^{hi}) and optionally positive for one or both of Oct4 and Sox2.

Whichever markers are ultimately chosen to identify or characterize the selected cell subpopulations, the actual monitoring, analysis and/or quantification may be conducted using any one of a number of standard techniques well known to one of skill in the art. For example, cell surface marker expression can be assayed by immunoassays including, but not limited to, western blots, immunohistochemistry, radioimmunoassays, enzyme-linked immunosorbent assay (ELISA) and ELISPOT based techniques, “sandwich” immunoassays, immunoprecipitation assays, precipitin reactions, gel diffusion precipitation reactions, immunodiffusion assays, agglutination assays, complement-fixation assays, immunoradiometric assays, fluorescent immunoassays, immunofluorescence, protein A immunoassays, laser capture microdissection, massively multiparametric mass cytometry, flow cytometry, mass cytometry, mass spectrometry, fluorescence activated cell sorting (FACS), fluorescence microscopy, magnetic cell separation, fluorescence based cell sorting using microfluidic systems, affinity separation, immunoaffinity adsorption based techniques such as affinity chromatography, magnetic particle separation, magnetic activated cell sorting or bead based cell sorting using microfluidic system, etc. and combinations thereof. In certain embodiments, the level or concentration of CD49f⁺ T-cells, or subtypes thereof as disclosed for example herein, in the T-cell population, may be determined by comparing the results to the level or concentration of CD49f⁺ T-cells, or subtypes thereof, in a reference T-cell population (e.g., a T-cell population that has a predetermined competence for immunotherapy, or a predetermined incompetence for immunotherapy) or to a predetermined reference range that correlates with competence or level of competence for immunotherapy, or with incompetence for immunotherapy.

In some embodiments, the T-cell population is determined to be competent for immunotherapy when the level or concentration of CD49f⁺ T-cells, or subtypes thereof, meets or exceeds a threshold level or concentration that correlates with competence for immunotherapy. In illustrative examples of this type, the T-cell population is determined to be competent for immunotherapy when the level or concentration of CD49f⁺ T-cells, or subtypes thereof disclosed for example herein, is 1% or more of the T-cells in the population, including 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or up to and including 100% of the T-cells in the population. In other illustrative examples, the T-cell population is determined to be competent for immunotherapy when the level or concentration of CD49f⁺ T-cells, or subtypes thereof disclosed for example herein, is 1% or more of the total number of cells in the population, including 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or up to and including 100% of the total number of cells in the T-cell population. In other embodiments, the T-cell population is determined to be incompetent for immunotherapy when the level or concentration of CD49f⁺ T-cells, or subtypes thereof disclosed for example herein, is below a threshold level or concentration that correlates with competence for immunotherapy. In non-limiting examples of this type, the T-cell population is determined to be incompetent for immunotherapy when the level or concentration of CD49f⁺ T-cells, or subtypes thereof disclosed for example herein, is less than 1% of the T-cells in the population, including less than 0.9%, less than 0.8%, less than 0.7%, less than 0.6%, less than 0.5%, less than 0.4%, less than 0.3%, less than 0.2% or less than 0.1% of the T-cells in the population. In other non-limiting examples, the T-cell population is determined to be incompetent for immunotherapy when the level or concentration of CD49f⁺ T-cells, or subtypes thereof disclosed for example herein, is less than 1% of the total number of cells in the population, including less than 0.9%, less than 0.8%, less than 0.7%, less than 0.6%, less than 0.5%, less than 0.4%, less than 0.3%, less than 0.2% or less than 0.1% of the total number of cells in the population. Suitably, the T-cell population is an unexpanded population of T-cells. Alternatively, the T-cell population is an expanded population of T-cells. In some of the same and other embodiments, the T-cell population results from a process that includes antigen-specific stimulation of T-cells to produce antigen-specific T-cells.

### 8. Kits for Assessing Competence of T-Cell Populations for Immunotherapy

Also disclosed herein are kits useful for determining competence of a T-cell population for immunotherapy, including adoptive cell therapy. In some embodiments, the kits include antigen-binding molecules or other binding partners, generally coupled to a label, for the monitoring, analysis and/or quantification using immunoassays, representative examples of which include western blots, immunohistochemistry, radioimmunoassays, enzyme-linked immunosorbent assay (ELISA) and ELISPOT based techniques, “sandwich” immunoassays, immunoprecipitation assays, precipitin reactions, gel diffusion precipitation reactions, immunodiffusion assays, agglutination assays, complement-fixation assays, immunoradiometric assays, fluorescent immunoassays, immunofluorescence, protein A immunoassays, laser capture microdissection, massively multiparametric mass cytometry, flow cytometry, mass cytometry, mass spectrometry, fluorescence activated cell sorting (FACS), fluorescence microscopy, magnetic cell separation, fluorescence based cell sorting using microfluidic systems, affinity separation, immunoaffinity adsorption based techniques such as affinity chromatography, magnetic particle separation, magnetic activated cell sorting or bead based cell sorting using microfluidic system, etc. and combinations thereof.

In some embodiments, the kit comprises an anti-CD49f antigen-binding molecule, and optionally one or more of anti-CD4, anti-CD8, anti-CD95, anti-CD27, anti-CD28, anti-CCR7, anti-CD45RA, anti-CD62L, and anti-CD127 antigen-binding molecules, coupled to a suitable label for use in the immunoassays.

In one embodiment, the kit comprises instructions to carry out monitoring, analysis and/or quantification in a sample, such as a T-cell sample, of CD49f, and optionally one or more of CD45RA, CCR7, CD95, CD28, CD27, CD62L, CD127, CD8 and CD4, using the antigen-binding molecules provided with the kit, to determine the level or concentration of cells that are positive for CD49f and optional one or more of the other markers in the T-cell sample. In some aspects, the instructions further include instructions to carry out one or more additional analysis steps, including comparing the level or concentration of cells that are positive for that marker in the T-cell sample to the level or concentration of cells that are positive for that marker in a reference T-cell population (e.g., a T-cell population that has a predetermined competence for immunotherapy, or a predetermined incompetence for immunotherapy) or to a predetermined reference range that correlates with competence or level of competence for immunotherapy, or with incompetence for immunotherapy.

### 9. Anti-CD49f Affinity Agent Therapy Embodiments

The present inventors have also determined that anti-CD49f affinity agents (e.g., an anti-CD49f antigen-binding molecules) that bind specifically to CD49f can be used to selectively stimulate activation of CD49f⁺ T-cells, resulting in significantly improved immune responses, including immune effector functions. Based on these findings, anti-CD49f affinity agents (e.g., an anti-CD49f antigen-binding molecules) are contemplated for use in enhancing immune effector function in patients having or at risk of developing an immune dysfunction, or requiring an augmented immune effector function, and/or for treating or inhibiting the development of a condition in a patient, wherein the patient has or is at risk of developing an immune dysfunction and/or is in need or desirous of an augmented immune effector function. In some embodiments, the anti-CD49f affinity agent (e.g., an anti-CD49f antigen-binding molecule) stimulates activation of CD49f⁺ T-cells subtypes including CD49f⁺ memory T-cells, representative examples of which include CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T- cells, CD49f⁺ CD27⁺ CD28⁺ CD95⁺ memory T-cells, CD49f⁺ CD27⁺ CD28⁺ CD45RA⁺ CD95⁺ memory T- cells, CD49f⁺ CD27⁺ CD28⁺ CD95⁺ CCR7⁺ memory T-cells and CD49f⁺ CD27⁺ CD28⁺ CD45RA⁺ CD95⁺ CCR7⁺ memory T-cells.

The anti-CD49f affinity agents disclosed herein include and encompass any molecule or moiety that binds specifically to CD49f. The affinity agent is suitably selected from antigen-binding molecules, illustrative examples of which include antibodies and non-antibody targeting molecules.

Antigen-binding molecule contemplated herein include, but are not limited to, antibodies, antigen-binding antibody fragments, or non-antibody targeting molecules that bind specifically to CD49f. The affinity agent may also encompass protein scaffolds whereby peptides with affinity for an antigen are embedded within the protein scaffold in a manner that allows the peptide(s) to be displayed and contact an epitope.

Antibodies contemplated by the present invention include whole antibodies, including polyclonal and monoclonal antibodies, and antigen-binding antibody fragments. Thus, antibodies may be selected from naturally occurring antibodies that comprise at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as VL) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen or epitope thereof. The constant regions of the antibodies may mediate the binding of the immunoglobulin to host tissues or factors, including various cells of the immune system (e.g., effector cells) and the first component (Clq) of the classical complement system. Non-limiting examples of antibodies include monoclonal antibodies, human antibodies, humanized antibodies, camelized antibodies, chimeric antibodies, bi-specific or multiple-specific antibody and anti-idiotypic (anti-Id) antibodies. The antibodies can be of any isotype (e.g., IgG, IgE, IgM, IgD, IgA and IgY), class (e.g., IgG1, IgG2, IgG3, IgG4, IgA1 and IgA2) or subclass.

Generally, antibody fragments include portions of an antibody. In some embodiments, these portions are part of the contact domain(s) of an antibody. In some other embodiments, these portion(s) are antigen-binding fragments that retain the ability to specifically bind with an epitope. Examples of binding fragments include, but are not limited to, single-chain Fv (scFv), Fab fragments, monovalent fragments consisting of the V_{L}, V_{H}, C_{L} and C_{H1} domains; a F(ab)₂ fragment, bivalent fragments comprising two Fab fragments linked together by a disulfide bridge at the hinge region; Fd fragments consisting of the VH and CH1 domains; a Fv fragment consisting of the V_{L} and V_{H} domains of a single arm of an antibody; dAb fragments (Ward et al., 1989. Nature 341:544-546), which consists of a V_{H} domain; and an isolated complementarity determining region (CDR). Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23: 1126-1136). Antibody fragments can be incorporated into single chain molecules comprising a pair of tandem Fv segments (V_{H}-C_{H1}-V_{H}-C_{H1}) which, together with complementary light chain polypeptides, form a pair of antigen binding regions (as disclosed, e.g., Zapata et al. (1995. Protein Eng. 8:1057-1062); and U.S. Pat. No. 5,641,870). In some embodiments, the affinity agent is a monoclonal antibody that binds specifically to CD49f.

Numerous methods of preparing antibodies to antigens of interest are known in the art. For example, monoclonal antibodies to CD49f can be made using conventional hybridoma methods that are often based on the seminal method of Kohler, G. et al. (1975, “Continuous Cultures Of Fused Cells Secreting Antibody Of Predefined Specificity,” Nature 256:495-497) or a modification thereof. Typically, monoclonal antibodies are developed in non-human species, such as mice. In general, a mouse or rat is used for immunization but other animals may also be used. The antibodies may be produced by immunizing mice with an immunogenic amount of an immunogen, in this case a chimeric polypeptide or complex of the present invention. The immunogen may be administered multiple times at periodic intervals such as, bi weekly, or weekly, or may be administered in such a way as to maintain viability in the animal.

To monitor the antibody response, a small biological sample (e.g., blood) may be obtained from the animal and tested for antibody titer against the immunogen. The spleen and/or several large lymph nodes can be removed and dissociated into single cells. If desired, the spleen cells may be screened (after removal of non-specifically adherent cells) by applying a cell suspension to a plate or to a well coated with the antigen. B-cells, expressing membrane-bound immunoglobulin specific for the antigen, will bind to the plate, and are not rinsed away with the rest of the suspension. Resulting B-cells, or all dissociated spleen cells, can then be fused with myeloma cells (e.g., X63-Ag8.653 and those from the Salk Institute, Cell Distribution Center, San Diego, Calif.). Polyethylene glycol (PEG) may be used to fuse spleen or lymphocytes with myeloma cells to form a hybridoma. The hybridoma is then cultured in a selective medium (e.g., hypoxanthine, aminopterin, thymidine medium, otherwise known as “HAT medium”). The resulting hybridomas are then plated by limiting dilution, and are assayed for the production of antibodies that bind specifically to the immunogen, using, for example, FACS (fluorescence activated cell sorting) or immunohistochemistry (IHC) screening. The selected monoclonal antibody-secreting hybridomas are then cultured either in vitro (e.g., in tissue culture bottles or hollow fiber reactors), or in vivo (e.g., as ascites in mice).

As another alternative to the cell fusion technique, Epstein-Barr Virus (EBV)-immortalized B cells may be used to produce monoclonal antibodies that are immuno-interactive with a subject chimeric polypeptide or complex. The hybridomas are expanded and subcloned, if desired, and supernatants are assayed for anti-immunogen activity by conventional assay procedures (e.g., FACS, IHC, radioimmunoassay, enzyme immunoassay, fluorescence immunoassay, etc.).

Thus, the present disclosure also contemplates methods of producing an antigen-binding molecule that is immuno-interactive with CD49f, wherein the method comprises: (1) immunizing an animal with a CD49f polypeptide or portion thereof; (2) isolating a B cell from the animal, which is immuno-interactive with CD49f; and (3) producing the antigen-binding molecule expressed by that B cell. The present disclosure also encompasses antigen-binding molecule that are produced by such methods as well as derivatives thereof. Also encompassed are cells including hybridomas that are capable of producing the antigen-binding molecules of the invention, and methods of producing antigen-binding molecules from those cells. In specific embodiments, the antigen-binding molecules produced by the methods and cells of the invention are preferably neutralizing antigen-binding molecules.

Also contemplated are chimeric antibodies and humanized antibodies. In some embodiments, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557, Padlan et al., 1995, FASEB J. 9:133-39, Tamura et al., 2000, J. Immunol. 164:1432-41, Zhang, W., et al., Molecular Immunology 42(12):1445-1451, 2005; Hwang W. et al., Methods 36(1):35-42, 2005; Dall'Acqua W F, et al., Methods 36(1):43-60, 2005; and Clark, M., Immunology Today 21(8):397-402, 2000.

An antibody of the present disclosure may also be a fully human monoclonal antibody. Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue.

Antibodies produced in the animal incorporate human immunoglobulin polypeptide chains encoded by the human genetic material introduced into the animal. In one embodiment, a non-human animal, such as a transgenic mouse, is immunized with a subject chimeric polypeptide or complex immunogen.

Examples of techniques for production and use of transgenic animals for the production of human or partially human antibodies are described in U.S. Pat. Nos. 5,814,318, 5,569,825, and 5,545,806, Davis et al., Production of human antibodies from transgenic mice in Lo, ed. Antibody Engineering: Methods and Protocols, Humana Press, NJ: 191-200 (2003), Kellermann et al., 2002, Curr Opin Biotechnol. 13:593-97, Russel et al., 2000, Infect Immun. 68:1820-26, Gallo et al., 2000, Eur J. Immun. 30:534-40, Davis et al., 1999, Cancer Metastasis Rev. 18:421-25, Green, 1999, J Immunol Methods 231:11-23, Jakobovits, 1998, Advanced Drug Delivery Reviews 31:33-42, Green et al., 1998, J Exp Med. 188:483-95, Jakobovits A, 1998, Exp. Opin. Invest. Drugs 7:607-14, Tsuda et al., 1997, Genomics 42:413-21, Mendez et al., 1997, Nat. Genet. 15:146-56, Jakobovits, 1994, Curr Biol. 4:761-63, Arbones et al., 1994, Immunity 1:247-60, Green et al., 1994, Nat. Genet. 7:13-21, Jakobovits et al., 1993, Nature 362:255-58, Jakobovits et al., 1993, Proc Natl Acad Sci USA 90:2551-55. Chen, J., M. et a. Int. Immunol. 5 (1993): 647-656, Choi et al., 1993, Nature Genetics 4: 117-23, Fishwild et al., 1996, Nature Biotech. 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., 1995, Int. Rev. Immunol. 13: 65-93, Neuberger, 1996, Nature Biotech. 14: 826, Taylor et al., 1992, Nucleic Acids Research 20: 6287-95, Taylor et al., 1994, Int. Immunol. 6: 579-91, Tomizuka et al., 1997, Nature Genetics 16: 133-43, Tomizuka et al., 2000, Proc Natl Acad Sci USA 97: 722-27, Tuaillon et al., 1993, Proc Natl Acad Sci USA 90: 3720-24, and Tuaillon et al., 1994, J. Immunol. 152: 2912-20; Lonberg et al., Nature 368:856, 1994; Taylor et al., Int. Immuno. 6:579, 1994; U.S. Pat. No. 5,877,397; Bruggemann et al., 1997 Curr. Opin. Biotechnol. 8:455-58; Jakobovits et al., 1995. Ann. N.Y. Acad. Sci. 764:525-35. In addition, protocols involving the XenoMouse®. (Abgenix, now Amgen, Inc.) are described, for example in U.S. Ser. No. 05/011,8643 and WO 05/694879, WO 98/24838, WO 00/76310, and U.S. Pat. No. 7,064,244.

The present invention further encompasses fragments of an anti-CD14 antibody of the invention. Such fragments can consist entirely of antibody-derived sequences or can comprise additional sequences. Examples of antigen-binding fragments include Fab, F(ab′)₂, single chain antibodies, diabodies, triabodies, tetrabodies, and domain antibodies. Other examples are provided in Lunde et al., 2002, Biochem. Soc. Trans. 30:500-06.

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol. Biol. 178:379-87.

Antigen binding fragments derived from an antibody can also be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 89:230, 1960; Porter, Biochem. J. 73:119, 1959; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003), pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

Another form of an antibody fragment is a peptide comprising one or more complementarity determining regions (CDRs) of an antibody. CDRs can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). The antibody fragment further may comprise at least one variable region domain of an antibody described herein. Thus, for example, the V region domain may be monomeric and be a V_{L} and V_{H} domain, which is capable of independently binding a subject ectodomain polypeptide or complex with an affinity at least equal to 10⁻⁷ M or less.

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C-terminal amino acid to at least one other antibody domain or a fragment thereof. Thus, for example, a V_{H} domain that is present in the variable region domain may be linked to an immunoglobulin CH1 domain, or a fragment thereof. Similarly a V_{L} domain may be linked to a C_{K} domain or a fragment thereof. In this way, for example, the antibody may be a Fab fragment wherein the antigen binding domain contains associated V_{H} and V_{L} domains covalently linked at their C-termini to a CH1 and C_{K} domain, respectively. The CH1 domain may be extended with further amino acids, for example to provide a hinge region or a portion of a hinge region domain as found in a Fab′ fragment, or to provide further domains, such as antibody CH2 and CH3 domains.

In some embodiments, the anti-CD49f affinity agent is a nanobody. Nanobodies are single-domain antibodies of about 12-15 kDa in size (about 110 amino acids in length) and can selectively bind to target antigens, like full-size antibodies, and have similar affinities for antigens. However, because of their much smaller size, they may be capable of better penetration into tissues. The smaller size also contributes to the stability of the nanobody, which is more resistant to pH and temperature extremes than full size antibodies (Van Der Linden et al., 1999. Biochim Biophys Acta 1431:37-46). Single-domain antibodies were originally developed following the discovery that camelids (camels, alpacas, llamas) possess fully functional antibodies without light chains (e.g., Hamsen et al., 2007. Appl Microbiol Biotechnol. 77:13-22). The heavy-chain antibodies consist of a single variable domain (Van) and two constant domains (C_{H2} and C_{H3}). Like antibodies, nanobodies may be developed and used as multivalent and/or bispecific constructs. The plasma half-life of nanobodies is shorter than that of full-size antibodies, with elimination primarily by the renal route. Because they lack an Fc region, they do not exhibit complement dependent cytotoxicity. Nanobodies may be produced by immunization of camels, llamas, alpacas or sharks with target antigens such as polymer chains, following by isolation of mRNA, cloning into libraries and screening for antigen binding. Nanobody sequences may be humanized by standard techniques (e.g., Jones et al., 1986. Nature 321:522, Riechmann et al., 1988. Nature 332:323, Verhoeyen et al., 1988. Science 239:1534, Carter et al., 1992. Proc Natl Acad Sci. USA 89:4285, Sandhu, 1992. Crit. Rev. Biotech. 12:437, Singer et al., 1993, J. Immun. 150:2844). Humanization is relatively straightforward because of the high homology between camelid and human FR sequences.

In certain embodiments, the affinity agents disclosed herein may comprise one or more avimer sequences. Avimers are a class of binding proteins somewhat similar to antibodies in their affinities and specificities for various target molecules. They were developed from human extracellular receptor domains by in vitro exon shuffling and phage display. (Silverman et al., 2005. Nat. Biotechnol. 23:1493-94; Silverman et al., 2006. Nat. Biotechnol. 24:220). The resulting multidomain proteins may comprise multiple independent binding domains that may exhibit improved affinity (in some cases sub-nanomolar) and specificity compared with single-epitope binding proteins. Additional details concerning methods of construction and use of avimers are disclosed, for example, in U.S. Pat. Appl. Pub. Nos. 20040175756, 20050048512, 20050053973, 20050089932 and 20050221384, the Examples section of each of which is incorporated herein by reference.

Certain embodiments of affinity agents relate to binding peptides and/or peptide mimetics of various polymer groups. Binding peptides may be identified by any method known in the art, including but not limiting to the phage display technique. Various methods of phage display and techniques for producing diverse populations of peptides are well known in the art. For example, U.S. Pat. Nos. 5,223,409; 5,622,699 and 6,068,829 disclose methods for preparing a phage library. The phage display technique involves genetically manipulating bacteriophage so that small peptides can be expressed on their surface (Smith and Scott, 1985, Science 228:1315-1317; Smith and Scott, 1993, Meth. Enzymol. 21:228-257). In addition to peptides, larger protein domains such as single-chain antibodies may also be displayed on the surface of phage particles (Arap et al., 1998, Science 279:377-380). In some embodiments, anti-CD49f binding peptides corresponding to laminin may be used as the affinity agent. In this regard, it is known that CD49f is a receptor for laminin.

In certain embodiments, an affinity agent may be an aptamer. Methods of constructing and determining the binding characteristics of aptamers are well known in the art. For example, such techniques are described in U.S. Pat. Nos. 5,582,981, 5,595,877 and 5,637,459, the Examples section of each incorporated herein by reference. Methods for preparation and screening of aptamers that bind to particular targets of interest are well known, for example U.S. Pat. Nos. 5,475,096 and 5,270,163, the Examples section of each incorporated herein by reference. Aptamers may be prepared by any known method, including synthetic, recombinant, and purification methods, and may be used alone or in combination with other ligands specific for the same target. In general, a minimum of approximately 3 nucleotides, preferably at least 5 nucleotides, are necessary to effect specific binding. Aptamers of sequences shorter than 10 bases may be feasible, although aptamers of 10, 20, 30 or 40 nucleotides may be preferred. Aptamers may be isolated, sequenced, and/or amplified or synthesized as conventional DNA or RNA molecules. Alternatively, aptamers of interest may comprise modified oligomers. Any of the hydroxyl groups ordinarily present in aptamers may be replaced by phosphonate groups, phosphate groups, protected by a standard protecting group, or activated to prepare additional linkages to other nucleotides, or may be conjugated to solid supports. One or more phosphodiester linkages may be replaced by alternative linking groups, such as P(O)O replaced by P(O)S, P(O)NR₂, P(O)R, P(O)OR′, CO, or CNR₂, wherein R is H or C₁-C₂₀ alkyl and R′ is C₁-C₂₀ alkyl; in addition, this group may be attached to adjacent nucleotides through O or S, Not all linkages in an oligomer need to be identical.

Certain alternative embodiments may utilize affibodies in place of antibodies. Affibodies are commercially available from Affibody AB (Solna, Sweden). Affibodies are small proteins that function as antibody mimetics and are of use in binding target molecules including affinity agent-binding partners on the polymer chains. Affibodies were developed by combinatorial engineering on an alpha helical protein scaffold (Nord et al., 1995. Protein Eng. 8:601-8; Nord et al., 1997. Nat Biotechnol. 15:772-77). The affibody design is based on a three-helix bundle structure comprising the IgG binding domain of protein A (Nord et al., 1995; 1997). Affibodies with a wide range of binding affinities may be produced by randomization of thirteen amino acids involved in the Fc binding activity of the bacterial protein A (Nord et al., 1995; 1997). After randomization, the PCR amplified library was cloned into a phagemid vector for screening by phage display of the mutant proteins. The phage display library may be screened against any known antigen, including polymer chains and their moieties, using standard phage display screening techniques (e.g., Pasqualini and Ruoslahti, 1996. Nature 380:364-366; Pasqualini, 1999. Quart. J. Nucl. Med. 43:159-162), in order to identify one or more affibodies against CD49f.

Fynomers can also bind to target antigens with a similar affinity and specificity to antibodies. Fynomers are based on the human Fyn SH3 domain as a scaffold for assembly of binding molecules. The Fyn SH3 domain is a fully human, 63-aa protein that can be produced in bacteria with high yields. Fynomers may be linked together to yield a multispecific binding protein with affinities for two or more different antigen targets. Fynomers are commercially available from COVAGEN AG (Zurich, Switzerland).

In some embodiments, the anti-CD49f affinity agent also has specificity for at least one other target and thus defines a multi-specific targeting construct. Accordingly, the present disclosure further contemplates a multi-specific targeting construct comprising an affinity agent that binds specifically to CD49f and a targeting ligand that targets the multi-specific agent to a target site. In this context, the targeting ligand targets the targeting construct to, and generally has specificity for the target site, which is suitably a binding partner of the ligand. The binding partner may be a molecule or macromolecule of a cell, a soluble molecule or a soluble macromolecule. The targeting ligand may be synthetic, semi-synthetic, or naturally occurring. Materials or substances which may serve as targeting ligands include, for example, proteins, including antigen-binding molecules as described for example above, hormones, hormone analogues, glycoproteins and lectins, peptides, polypeptides, amino acids, sugars, saccharides, including monosaccharides and polysaccharides, carbohydrates, small molecules, vitamins, steroids, steroid analogs, hormones, cofactors, bioactive agents, and genetic material, including nucleosides, nucleotides, nucleotide acid constructs and polynucleotides.

The targeting ligand may be selected from affinity agents (e.g., antibodies, antigen-binding antibody fragments, or non-antibody targeting molecules), cytokines, chemokines, growth factors (e.g., granulocyte colony stimulating factor (G-CSF), granulocyte macrophage colony stimulating factor (GM-CSF), epidermal growth factor (EGF), fibroblast growth factor (FGF), keratinocyte growth factor (KGF)), interferons, erythropoietin (EPO), TNF-α, interleukins, integrins, immunoglobulins, hormones (e.g., insulin, gonadotropins, growth hormone) and hormone analogues, peptides, transferrin, proteins that interact with a cell surface molecule or with a pattern recognition receptor, tumor receptor binding molecules, and molecules involved in vascular lesions, amino acids, sugars, saccharides, including monosaccharides and polysaccharides, carbohydrates, glycoproteins, lectins, small molecules, including drugs, vitamins, steroids, steroid analogs, cofactors, bioactive agents, and genetic material, including nucleosides, nucleotides, nucleic acid constructs and polynucleotides. In specific embodiments, the targeting ligand is an scFv.

Ligand-mediated targeting to specific tissues through binding to their respective receptors on the cell surface offers an attractive approach to improve the tissue-specific delivery of payloads. Specific targeting to disease-relevant cell types and tissues may help to lower the effective dose, reduce side effects and consequently maximize the therapeutic index. Carbohydrates and carbohydrate clusters with multiple carbohydrate motifs represent an important class of targeting ligands, which allow the targeting of drugs to a wide variety of tissues and cell types. For examples, see Hashida, et al., 2001. Adv Drug Deliv Rev. 52:187-9; Monsigny et al., 1994. Adv Drug De/iv Rev. 14:1-24; Gabius et al., 1996. Eur J Pharm and Biopharm 42:250-261; Wadhwa and Rice, 1995. J Drug Target. 3:111-127. Carbohydrate based targeting ligands include, but are not limited to, D-galactose, multivalent galactose, N-acetyl-D-galactose (GalNAc), multivalent GalNAc, e.g. GalNAC2 and GalNAc3; D-mannose, multivalent mannose, multivalent lactose, N-acetyl-galactosamine, N-acetyl-glucosamine, multivalent fucose, glycosylated polyaminoacids and lectins. The term multivalent indicates that more than one monosaccharide unit is present. Such monosaccharide subunits may be linked to each other through glycosidic linkages or linked to a scaffold molecule.

Lipophilic moieties, such as cholesterol or fatty acids can substantially enhance plasma protein binding and consequently circulation half-life. In addition, binding to certain plasma proteins, such as lipoproteins, has been shown to increase uptake in specific tissues expressing the corresponding lipoprotein receptors (e.g., LDL-receptor or the scavenger receptor SR-B1). For examples, see Bijsterbosch et al., 2000. Nucleic Acids Res. 28:2717-25; Wolfrum et al., 2007). Nat Biotechnol. 25:1149-57. Exemplary lipophilic moieties that enhance plasma protein binding include, but are not limited to, sterols, cholesterol, fatty acids, cholic acid, lithocholic acid, dialkylglycerides, diacylglyceride, phospholipids, sphingolipids, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyDlithocholic acid, O3-(oleoyl)cholenic acid, dimethoxytrityl, phenoxazine, aspirin, naproxen, ibuprofen, vitamin E and biotin etc.

Folates represent another class of ligands, which has been widely used for targeted drug delivery via the folate receptor. This receptor is highly expressed on a wide variety of tumor cells, as well as other cells types, such as activated macrophages. For examples, see Matherly and Goldman, 2003. Vitamins Hormones 66:403-456; Sudimack and Lee, 2000. Adv Drug Delivery Rev. 41:147-162. Similar to carbohydrate-based ligands, folates have been shown to be capable of delivering a wide variety of drugs, including nucleic acids and even liposomal carriers. For examples, see Reddy et al., 1999. J Pharm Sci. 88:1112-1118; Lu and Low, 2002. Adv Drug Delivery Rev. 54:675-693.

The targeting ligands can also include other receptor binding ligands such as hormones and hormone receptor binding ligands. A targeting ligand can be a thyrotropin, melanotropin, lectin, glycoprotein, surfactant protein A, mucin, glycosylated polyaminoacids, transferrin, bisphosphonate, polyglutamate, polyaspartate, a lipid, folate, vitamin B12, biotin, or an aptamer.

The targeting ligands also include proteins, peptides and peptidomimetics that bind with a target site. A peptidomimetic is a molecule capable of folding into a defined three-dimensional structure similar to a natural peptide. The peptide or peptidomimetic moiety can be about 5-50 amino acids long, e.g., about 5, 10, 15, 20, 25, 30, 35, 40, 45, or 50 amino acids long Such peptides include, but are not limited to, RGD containing peptides and peptidomimetics that can target cancer cells, in particular cells that exhibit αᵥβ₃ integrin. Targeting peptides can be linear or cyclic, and include D-amino acids, non-peptide or pseudo-peptide linkages, peptidyl mimics. In addition the peptide and peptide mimics can be modified, e.g., glycosylated or methylated. Synthetic mimics of targeting peptides are also included.

In specific embodiments, the targeting ligands bind with target binding partners selected from: carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD47, CD52, CD54, CD55, CD59, CD64, CD66a-e, CD67, CD70, CD70L, CD72, CD74, CD79a, CD79b, CD80, CD83, CD95, CD126, CD133, CD138, CD147, CD154, CD171, CD200, AFP, PSMA, CEACAM5, CEACAM-6, c-MET, B7, ED-B of fibronectin, Factor H, FHL-1, Flt-3, folate receptor, GROB, histone H2B, histone H3, histone H4, HMGB-1, hypoxia inducible factor (HIF), HM1.24, insulin-like growth factor-1 (ILGF-1), IFNγ, IFN-α, IFN-α, IL-2, IL-4R, IL-6R, IL-13R, IL-15R, IL-17R, IL-18R, IL-6, IL-8, IL-12, IL-15, IL-17, IL-18, IL20Rα, IL-23, IL-25, IP-10, LIV-1, MAGE, mCRP, MCP-1, MIP-1A, MIP-1B, MIF, MUC1, MUC2, MUC3, MUC4, MUC5, MUC5a,c, MUC16, PAM4 antigen, NCA-95, NCA-90, Ia, HM1.24, EGP-1 (TROP-2), EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGFR, EGFR, FGFR, P1GF, complement factors C3, C3a, C3b, C5a, C5, and an oncogene product, B7, Ia, Ii, HMI.24, HLA-DR (e.g., HLA-DR10), NCA95, NCA90, HCG and sub-units, CEA (CEACAM5), CEACAM-6, CSAp, EGP-I, EGP-2, Ba 733, KC4 antigen, KS-I antigen, KS1-4, Le-Y, PlGF, ED-B fibronectin, NCA 66a-d, PAM-4 antigen, PSA, PSMA, RS5, SIOO, TAG-72, TIOI, TAG TRAIL-RI, TRAIL-R2, p53, tenascin, insulin growth factor-1 (IGF-I), Tn antigen, bone morphogenetic protein receptor-type IB (BMPRiB), E16, six transmembrane epithelial antigen of prostate (STEAPi), megakaryocyte potentiating factor (MPF), type II sodium-dependent phosphate transporter 3b (Napi3b), Semaphorin 5b (Sema 5b), PSCA h1g, Endothelin type B receptor (ETBR), MSG783, six transmembrane epithelial antigen of prostate 2 (STEAP2), transient receptor potential cation channel subfamily M, member 4 (TrpM4), teratocarcinoma-derived growth factor (CRIPTO), Fc receptor-like protein 2 (FcRH2), HER2, Epidermal growth factor receptor (EGFR) Brevican, Ephb2R, ASLG659, PSCA, GEDA, B cell-activating factor receptor (BAFF-R), CXCR5, HLA-DOB, Purinergic receptor P2X ligand-gated ion channel 5 (P2X5), Lymphocyte antigen 64 (LY64), Fc receptor-like protein 1 (FcRH1), Immunoglobulin superfamily receptor translocation associated 2 (IRTA2), a matrix metalloproteinase, oxidized LDL, scavenger receptor A, CD36, CD68, lectin-like oxidized LDL receptor-1 (LOX-1), SR-A1 and SR-B1, and/or molecules expressed by pathogens such as Epstein-Barr virus (EBV), cytomegalovirus (CMV), human immunodeficiency virus (HIV), hepatitis C virus (HCV), hepatitis B virus (HBV) or other pathogens.

In specific embodiments, the target-binding partner is a cell surface antigen, which suitably undergoes internalization, such as a protein, sugar, lipid head group or other antigen on the cell surface. In representative examples of this type, a payload associated with the targeting construct modulates (e.g., interferes) with cellular processes or images the cell. In some embodiments, therefore, a targeting construct of the present invention binds with a cell surface antigen through its targeting ligand and the targeting construct is internalized into the cell. Suitably, the internalization is mediated by endocytosis. In some embodiments, binding of the targeting construct with the cell surface antigen detectably agonizes or antagonizes an activity of the cell surface antigen. In some embodiments, binding of the targeting construct with the cell surface antigen detectably agonizes or antagonizes an intracellular pathway. In some embodiments, binding of the targeting construct with the cell surface antigen inhibits proliferation, survival or viability of a cell with which the cell surface antigen is associated.

A large number of antibodies against various disease targets, including but not limited to tumor-associated antigens, have been deposited at various depository institutions including for example the American Type Culture Collection (ATCC, Manassas, Va.) ATCC and/or have published variable region sequences and are available for use in the preparation of targeting ligands. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206; 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953; 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art (see, e.g., U.S. Pat. Nos. 7,531,327; 7,537,930; 7,608,425 and 7,785,880, the Examples section of each of which is incorporated herein by reference).

In specific embodiments, the antibodies or antibody fragments used as the targeting ligands are specific for cancer antigens. Particular antibodies that may be of use for therapy of cancer within the scope of the present invention include, but are not limited to, LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD-1 receptor), nivolumab (anti-PD-1 receptor), ipilimumab (anti-CTLA-4), RS7 (anti-epithelial glycoprotein-1 (EGP-1, also known as TROP-2)), PAM4 or KC4 (both anti-mucin), MN-14 (anti-carcinoembryonic antigen (CEA, also known as CD66e or CEACAM5), MN-15 or MN-3 (anti-CEACAM6), Mu-9 (anti-colon-specific antigen-p), Immu 31 (an anti-alpha-fetoprotein), R1 (anti-IGF-1R), A19 (anti-CD19), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin) and trastuzumab (anti-ErbB2). Such antibodies are known in the art (e.g., U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20050271671; 20060193865; 20060210475; 20070087001; the Examples section of each incorporated herein by reference.) Specific known antibodies of use include hPAM4 (U.S. Pat. No. 7,282,567), hA20 (U.S. Pat. No. 7,251,164), hA19 (U.S. Pat. No. 7,109,304), hIMMU-31 (U.S. Pat. No. 7,300,655), hLL1 (U.S. Pat. No. 7,312,318), hLL2 (U.S. Pat. No. 7,074,403), hMu-9 (U.S. Pat. No. 7,387,773), hL243 (U.S. Pat. No. 7,612,180), hMN-14 (U.S. Pat. No. 6,676,924), hMN-15 (U.S. Pat. No. 7,541,440), hR1 (U.S. patent application Ser. No. 12/772,645), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575) the text of each recited patent or application is incorporated herein by reference with respect to the Figures and Examples sections.

Other useful antigens that may be targeted include carbonic anhydrase IX, B7, CCCL19, CCCL21, CSAp, HER-2/neu, BrE3, CD1, CD11a, CD2, CD3, CD4, CD5, CD8, CD11A, CD14, CD15, CD16, CD18, CD19, CD20 (e.g., C2B8, hA20, 1F5 MAbs), CD21, CD22, CD23, CD25, CD29, CD30, CD32b, CD33, CD37, CD38, CD40, CD40L, CD44, CD45, CD46, CD47, CD52, CD54, CD55, CD59, CD64, CD67, CD70, CD74, CD79a, CD80, CD83, CD95, CD126, CD133, CD138, CD147, CD154, CEACAM5, CEACAM6, CTLA-4, alpha-fetoprotein (AFP), VEGF (e.g., AVASTIN®, fibronectin splice variant), ED-B fibronectin (e.g., L19), EGP-1 (TROP-2), EGP-2 (e.g., 17-1A), EGF receptor (ErbB1) (e.g., ERBITUX), ErbB2, ErbB3, Factor H, FHL-1, Flt-3, folate receptor, Ga 733, GRO-.beta., HMGB-1, hypoxia inducible factor (HIF), HM1.24, HER-2/neu, histone H2B, histone H3, histone H4, insulin-like growth factor (ILGF), IFN-γ, IFN-α, IFN-D, IFN-A, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, the HLA-DR antigen to which L243 binds, CD66 antigens, i.e., CD66a-d or a combination thereof, MAGE, mCRP, MCP-1, MIP-1A, MIP-1B, macrophage migration-inhibitory factor (MIF), MUC1, MUC2, MUC3, MUC4, MUC5ac, placental growth factor (P1GF), PSA (prostate-specific antigen), PSMA, PAM4 antigen, PD-1 receptor, PD-L1, NCA-95, NCA-90, A3, A33, Ep-CAM, KS-i, Le(y), mesothelin, S100, tenascin, TAC, Tn antigen, Thomas-Friedenreich antigens, tumor necrosis antigens, tumor angiogenesis antigens, TNF-α, TRAIL receptor (R1 and R2), TROP-2, VEGFR, RANTES, T101, as well as cancer stem cell antigens, complement factors C3, C3a, C3b, C5a, C5, and an oncogene product.

For multiple myeloma therapy, suitable targeting antibodies have been described against, for example, CD38 and CD138 (Stevenson, 2006. Mol Med. 12(11-12):345-346; Tassone et al., 2004. Blood 104(12):3688-96), CD74 (Stein et al., 2007. Clin Cancer Res. 13(18 Pt 2):5556s-5563s), CS1 (Tai et al., 2008. Blood 112(4):1329-37, and CD40 (Tai et al., 2005. Cancer Res. 65(13):5898-5906).

Macrophage migration inhibitory factor (MIF) is an important regulator of innate and adaptive immunity and apoptosis. It has been reported that CD74 is the endogenous receptor for MIF (Leng et al., 2003. J Exp Med 197:1467-76). The therapeutic effect of antagonistic anti-CD74 antibodies on MIF-mediated intracellular pathways may be of use for treatment of a broad range of disease states, such as cancers of the bladder, prostate, breast, lung, colon and chronic lymphocytic leukemia (e.g., Meyer-Siegler et al., 2004. BMC Cancer 12:34; Shachar and Haran, 2011. Leuk Lymphoma 52:1446-54); autoimmune diseases such as rheumatoid arthritis and systemic lupus erythematosus (Morand & Leech, 2005. Front Biosci 10:12-22; Shachar and Haran, 2011. Leuk Lymphoma 52:1446-54); kidney diseases such as renal allograft rejection (Lan, 2008. Nephron Exp Nephrol. 109:e79-83); and numerous inflammatory diseases (Meyer-Siegler et al., 2009. Mediators Inflamm epub Mar. 22, 2009; Takahashi et al., 2009. Respir Res 10:33; Milatuzumab (hLL1) is an exemplary anti-CD74 antibody of therapeutic use for treatment of MIF-mediated diseases.

Anti-TNF-α antibodies are known in the art and may be of use to treat immune diseases, such as autoimmune disease, immune dysfunction (e.g., graft-versus-host disease, organ transplant rejection) or diabetes. Known antibodies against TNF-α include the human antibody CDP571 (Ofei et al., 2011. Diabetes 45:881-85); murine antibodies MTNFα1, M2TNFAI, M3TNFAI, M3TNFABI, M302B and M303 (Thermo Scientific, Rockford, Ill.); infliximab (Centocor, Malvern, Pa.); certolizumab pegol (UCB, Brussels, Belgium); and Adalimumab (Abbott, Abbott Park, Ill.). These and many other known anti-TNF-α antibodies may be used as targeting ligands in the targeting constructs of the present invention. Other antibodies of use for therapy of immune dysregulatory or autoimmune disease include, but are not limited to, anti-B-cell antibodies such as veltuzumab, epratuzumab, milatuzumab or hL243; tocilizumab (anti-IL-6 receptor); basiliximab (anti-CD25); daclizumab (anti-CD25); efalizumab (anti-CD11a); muromonab-CD3 (anti-CD3 receptor); anti-CD40L (UCB, Brussels, Belgium); natalizumab (anti-.alpha.4 integrin) and omalizumab (anti-IgE).

Checkpoint inhibitor antibodies have been used primarily in cancer therapy. Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response against tumor tissues. Exemplary checkpoint inhibitor antibodies against cytotoxic T-lymphocyte antigen 4 (CTLA4, also known as CD152), programmed cell death protein 1 (PD 1, also known as CD279) and programmed cell death 1 ligand 1 (PD-L1, also known as CD274), may be used in combination with one or more other agents to enhance the effectiveness of immune response against disease cells, tissues or pathogens. Exemplary anti-PD1 antibodies include lambrolizumab (MK-3475, MERCK), nivolumab (BMS-936558, BRISTOL-MYERS SQUIBB), AMP-224 (MERCK), and pidilizumab (CT-011, CURETECH LTD.). Anti-PD1 antibodies are commercially available, for example from ABCAM® (AB137132), BIOLEGEND® (EH 12.2H7, RMP1-14) and AFFYMETRIX EBIOSCIENCE (J105, J116, MIH4). Exemplary anti-PD-L1 antibodies include MDX-1105 (MEDAREX), MEDI4736 (MEDIMMUNE) MPDL3280A (GENENTECH) and BMS-936559 (BRISTOL-MYERS SQUIBB). Anti-PD-L1 antibodies are also commercially available, for example from AFFYMETRIX EBIOSCIENCE (MIH1). Exemplary anti-CTLA4 antibodies include ipilimumab (Bristol-Myers Squibb) and tremelimumab (PFIZER). Anti-PD1 antibodies are commercially available, for example from ABCAM® (AB134090), SINO BIOLOGICAL INC. (11159-H03H, 11159-H08H), and THERMO SCIENTIFIC PIERCE (PA5-29572, PA5-23967, PA5-26465, MA1-12205, MA1-35914). Ipilimumab has recently received FDA approval for treatment of metastatic melanoma (Wada et al., 2013, J Transl Med 11:89).

Type-1 and Type-2 diabetes may be treated using known antibodies against B-cell antigens, such as CD22 (epratuzumab and hRFB4), CD74 (milatuzumab), CD19 (hA19), CD20 (veltuzumab) or HLA-DR (hL243) (see, e.g., Winer et al., 2011. Nature Med 17:610-18). Anti-CD3 antibodies also have been proposed for therapy of type-1 diabetes (Cernea et al., 2010. Diabetes Metab Rev. 26:602-05).

When two or more targeting ligands are present in a targeting construct, such targeting ligands may be the same or different. In non-limiting embodiments in which the targeting ligands of an individual construct are different, the binding partners of the ligands represent different cognate binding partners of a target complex (e.g., a heteropolymeric complex, including a heteromultimeric macromolecule such as a heteromultimeric polypeptide). In illustrative example of this type, a target complex represents a receptor that comprises at least two different polypeptide chains. Such target complexes include heterodimeric and heterotrimeric receptor complexes, illustrative examples of which include type I cytokine receptors that comprise different polypeptide chains, some of which are involved in ligand/cytokine interaction are generally referred to the a-chains and others that are involved in signal transduction which include the β- and γ-chains. Non-limiting examples of α-chains include the α-chains of the interleukin-2 receptor, interleukin-3 receptor, interleukin-4 receptor, interleukin-5 receptor, interleukin-6 receptor, interleukin-7 receptor, interleukin-9 receptor, interleukin-11 receptor, interleukin-12 receptor, interleukin-13 receptor, interleukin-15 receptor, interleukin-21 receptor, interleukin-23 receptor, interleukin-27 receptor, colony stimulating factor receptors, erythropoietin receptor, GM-CSF receptor, G-CSF receptor, hormone receptor/neuropeptide receptor, growth hormone receptor, prolactin receptor, oncostatin M receptor and leukemia inhibitory factor). The signal transducing chains are often shared between different receptors within this receptor family. For example, the IL-2 receptor common γ-chain (also known as CD132) is shared between: IL-2 receptor, IL-4 receptor, IL-7 receptor, IL-9 receptor, IL-13 receptor and IL-15 receptor. The common 3-chain (CD131 or CDw131) is shared between the following type I cytokine receptors: GM-CSF receptor, IL-3 receptor and IL-5 receptor. The gp230 receptor common γ-chain (also known as gp130, IL6ST, IL6-beta or CD130) is shared between: IL-6 receptor, IL-11 receptor, IL-12 receptor, IL-27 receptor, leukemia inhibitory factor receptor and Oncostatin M receptor. In certain strategies, it is desirable to bind specifically with the α-chain of a cytokine receptor and to signal through a to least one different signal-transducing chain, in order to alleviate for example certain unwanted side effects associated with signaling through the signal-transducing chain(s) normally associated with the heteromultimeric complex, as for example described in U.S. Pat. App. Pub. No. 20140140949, which is hereby incorporated by reference herein in its entirety. In these embodiments, one of the targeting ligands is adapted to bind preferentially with the α-chain and at least one other targeting ligand is adapted to bind one or more signal-transducing chains not normally associated with the α-chain.

### 10. Anti-CD49f Affinity Agent Therapeutic Combinations

Also contemplated herein are therapeutic combinations comprising an anti-CD49f affinity agent and at least one ancillary agent that stimulates immune effector function or that treats or inhibits the development of a condition in the patient, which is suitably selected from cancer, infectious disease, autoimmune disease, inflammatory disease, and immunodeficiency.

Ancillary agent encompassed by the present disclosure include an anti-pathogen agent or an anti-cancer agent. Anti-cancer agents, include, without limitation, 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g. prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide). In another embodiment, the compounds of the invention are administered in conjunction with anti-angiogenesis agents, such as antibodies to VEGF (e.g., bevacizumab (AVASTIN), ranibizumab (LUCENTIS)) and other promoters of angiogenesis (e.g., bFGF, angiopoietin-1), antibodies to alpha-v/beta-3 vascular integrin (e.g., VITAXIN), angiostatin, endostatin, dalteparin, ABT-510, CNGRC peptide TNF alpha conjugate, cyclophosphamide, combretastatin A4 phosphate, dimethylxanthenone acetic acid, docetaxel, lenalidomide, enzastaurin, paclitaxel, paclitaxel albumin-stabilized nanoparticle formulation (Abraxane), soy isoflavone (Genistein), tamoxifen citrate, thalidomide, ADH-1 (EXHERIN), AG-013736, AMG-706, AZD2171, sorafenib tosylate, BMS-582664, CHIR-265, pazopanib, PI-88, vatalanib, everolimus, suramin, sunitinib malate, XL184, ZD6474, ATN-161, cilenigtide, and celecoxib.

Suitable antiviral agents include, for example, virus-inactivating agents such as nonionic, anionic and cationic surfactants, and C31 G (amine oxide and alkyl betaine), polybiguanides, docosanol, acylcarnitine analogs, octyl glycerol, and antimicrobial peptides such as magainins, gramicidins, protegrins, and retrocyclins. Mild surfactants, e.g., sorbitan monolaurate, may advantageously be used as antiviral agents in the compositions described herein. Other antiviral agents that may advantageously be utilized in the compositions described herein include nucleotide or nucleoside analogs, such as tenofovir, acyclovir, amantadine, didanosine, foscarnet, ganciclovir, ribavirin, vidarabine, zalcitabine, and zidovudine. Further antiviral agents that may be used include non-nucleoside reverse transcriptase inhibitors, such as UC-781 (thiocarboxanilide), pyridinones, TIBO, nevaripine, delavirdine, calanolide A, capravirine and efavirenz. Other antiviral agents that may be used are those in the category of HIV entry blockers, such as cyanovirin-N, cyclodextrins, carregeenans, sulfated or sulfonated polymers, mandelic acid condensation polymers, monoclonal antibodies, chemokine receptor antagonists such as TAK-779, SCH-C/D, and AMD-3100, and fusion inhibitors such as T-20 and 1249.

Suitable antibacterial agents include antibiotics, such as aminoglycosides, cephalosporins, including first, second and third generation cephalosporins; macrolides, including erythromycins, penicillins, including natural penicillins, penicillinase-resistant penicillins, aminopenicillins, extended spectrum penicillins; sulfonamides, tetracyclines, fluoroquinolones, metronidazole and urinary tract antiseptics.

Suitable antifungal agents include amphotericin B, nystatin, griseofulvin, flucytosine, fluconazole, potassium iodide, intraconazole, clortrimazole, miconazole, ketoconazole, and tolnaftate.

Suitable antiprotozoal agents include antimalarial agents, such as chloroquine, primaquine, pyrimethamine, quinine, fansidar, and mefloquine; amebicides, such as dioloxamide, emetine, iodoquinol, metronidazole, paromomycine and quinacrine; pentamidine isethionate, atovaquone, and eflornithine.

The additional active agent may be an agent that treats or enhances the effect of a treatment against a symptom or side effect of a disease or treatment. In one embodiment, the additional active agent is an anti-inflammatory agent. Examples include, without limitation, H1-antihistamines (e.g., cetirizine), H2-antihistamines (e.g., ranitidine, famotidine), antileukotrienes (e.g., montelukast, zileuton), and nonsteroidal anti-inflammatory drugs.

The additional active agent may be an immunostimulatory agent and/or an immune checkpoint inhibitor that enhances the immunostimulatory effect of the fusion protein of the invention. Immunostimulatory agents include, without limitation, interleukin, interferon, cytokine, toll-like receptor (TLR) agonist, cytokine receptor agonist, CD40 agonist, Fc receptor agonist, CpG-containing immunostimulatory nucleic acid, complement receptor agonist, adjuvant, or CXCL12/CXCR4 axis inhibitors such as AMD3100, KRH-1636, T-20, T-22, T-140, TE-14011, T-14012, or TN14003, or an antibody that interferes with the dimerization of CXCR4. Immune checkpoint inhibitors include, without limitation, inhibitors of PD-1, PD-L1, CTLA4, B7-H3, B7-H4, BTLA, IDO, KIR, LAG3, A2AR, TIM-3, and VISTA, such as nivolumab, pembrolizumab, ipilimumab, durvalumab, or atezolizumab.

The therapeutic combination may comprise administering to the subject an additional therapy. The additional therapy may be any therapy known to be effective for treating a disease, e.g., therapies known to be effective for cancer treatment, e.g., surgery, radiotherapy, proton beam therapy, light-based therapy, etc.

### 11. Anti-CD49f Affinity Agent Compositions and Methods of Administration

Also disclosed herein are pharmaceutical compositions comprising an anti-CD49f affinity agent, formulated with one or more pharmaceutically-acceptable carriers. Optionally, the pharmaceutical composition comprises one or more other compounds, drugs, ingredients and/or materials. Regardless of the route of administration selected, the anti-CD49f affinity agent or therapeutic combinations of the present invention are formulated into pharmaceutically-acceptable dosage forms by conventional methods known to those of skill in the art (see, e.g., Remington, The Science and Practice of Pharmacy (21^{st} Edition, Lippincott Williams and Wilkins, Philadelphia, Pa.)).

The pharmaceutically acceptable carrier includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion).

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the anti-CD49f affinity agent or therapeutic combination is administered by intravenous infusion or injection. In another preferred embodiment, the anti-CD49f affinity agent or therapeutic combination is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically should be sterile and stable under the conditions of manufacture and storage. The composition can be formulated as a solution, microemulsion, dispersion, liposome, or other ordered structure suitable to high antigen-binding molecule concentration. Sterile injectable solutions can be prepared by incorporating the active compound (i.e., anti-CD49f affinity agent or therapeutic combination) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In specific embodiments, anti-CD49f affinity agent or a therapeutic combination as described herein may be conjugated to a vehicle for cellular delivery. In these embodiments, typically an anti-CD49f affinity agent of the disclosure, which may or may not be conjugated to a detectable label and/or ancillary therapeutic agent, is encapsulated in a suitable vehicle to either aid in the delivery of the anti-CD49f affinity agent or a therapeutic combination to target cells, to increase the stability of the affinity agent or therapeutic combination, or to minimize potential toxicity of the affinity agent or a therapeutic combination. As will be appreciated by a skilled artisan, a variety of vehicles are suitable for delivering an antibody of the present disclosure. Non-limiting examples of suitable structured fluid delivery systems may include nanoparticles, liposomes, microemulsions, micelles, dendrimers and other phospholipid-containing systems. Methods of incorporating antibodies into delivery vehicles are known in the art. Although various embodiments are presented below, it will be appreciate that other methods known in the art to incorporate an antigen-binding molecule or a therapeutic combination of the disclosure into a delivery vehicle are contemplated.

In some embodiments, a liposome delivery vehicle may be utilized. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the antigen-binding molecule or a therapeutic combination of the invention may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the anti-CD49f affinity agent of the disclosure (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In other embodiments, an anti-CD49f affinity agent or a therapeutic combination of the disclosure may be delivered to a cell as a microemulsion. Microemulsions are generally clear, thermodynamically stable solutions comprising an aqueous solution, a surfactant, and “oil”. The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The antibody of the disclosure may be encapsulated in a microemulsion by any method generally known in the art.

In yet other embodiments, an anti-CD49f affinity agent or a therapeutic combination of the present invention may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate antibodies of the disclosure therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

An anti-CD49f affinity agent or therapeutic combination of the disclosure can be administered by a variety of methods known in the art, although for many therapeutic applications, the preferred route/mode of administration is intravenous injection or infusion. In one embodiment, the anti-CD49f affinity agent or therapeutic combination is administered by intravenous infusion at a rate of more than 20 mg/min, e.g., 20-40 mg/min, and preferably greater than or equal to 40 mg/min to reach a dose of about 35 to 440 mg/m², preferably about 70 to 310 mg/m², and more preferably, about 110 to 130 mg/m². In another embodiment, the anti-CD49f affinity agent or therapeutic combination is administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, the anti-CD49f affinity agent or therapeutic combination can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Pharmaceutical compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for an effective amount of anti-CD49f affinity agent or therapeutic combination is 0.1-30 mg/kg, more preferably 1-25 mg/kg. Dosages and therapeutic regimens of the anti-CD49f affinity agent or therapeutic combination can be determined by a skilled artisan. In certain embodiments, the anti-CD49f affinity agent or therapeutic combination is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the anti-CD49f affinity agent or therapeutic combination is administered at a dose from about 10 to 20 mg/kg every other week.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include an effective amount of anti-CD49f affinity agent or therapeutic combination. The effective amount may be a “therapeutically effective amount” or a “prophylactically effective amount” of a anti-CD49f affinity agent or therapeutic combination of the invention. A “therapeutically effective amount” refers to an amount effective, at dosages and for periods of time necessary, to achieve the desired therapeutic result. A therapeutically effective amount of the anti-CD49f affinity agent or therapeutic combination may vary according to factors such as the disease state, age, sex, and weight of the individual, and the ability of the anti-CD49f affinity agent or therapeutic combination to elicit a desired response in the individual, such as but not limited to increased immune effector function, decreased immune effector dysfunction and increased responsiveness in immunotherapy. A therapeutically effective amount is also one in which any toxic or detrimental effects of the anti-CD49f affinity agent or therapeutic combination is outweighed by the therapeutically beneficial effects. A “therapeutically effective dosage” preferably inhibits a measurable parameter, e.g., tumor proliferation or tumor growth rate, or quantum of infection by at least about 20%, more preferably by at least about 40%, even more preferably by at least about 60%, and still more preferably by at least about 80% relative to untreated subjects. The ability of a compound to inhibit a measurable parameter, e.g., an infectious disease or cancer, can be evaluated in an animal model system predictive of efficacy in human infectious disease or cancers. Alternatively, this property of a composition can be evaluated by examining the ability of the compound to inhibit, for example in in vitro by assays known to the skilled practitioner.

By contrast, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

In order that the disclosure may be readily understood and put into practical effect, particular preferred embodiments will now be described by way of the following non-limiting examples.

### EXPERIMENTAL

### Functional Differentiation of Virus-Specific Human CD8⁺ T-Cells Correlates with Level of CD49f Expression

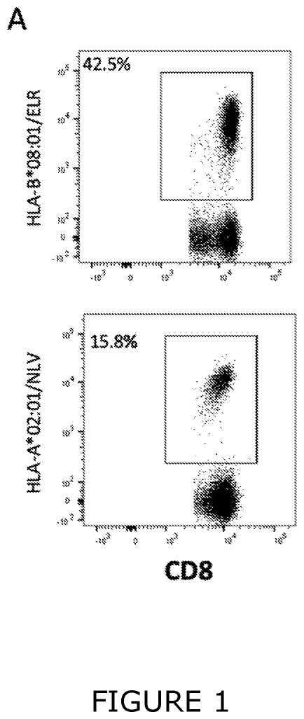

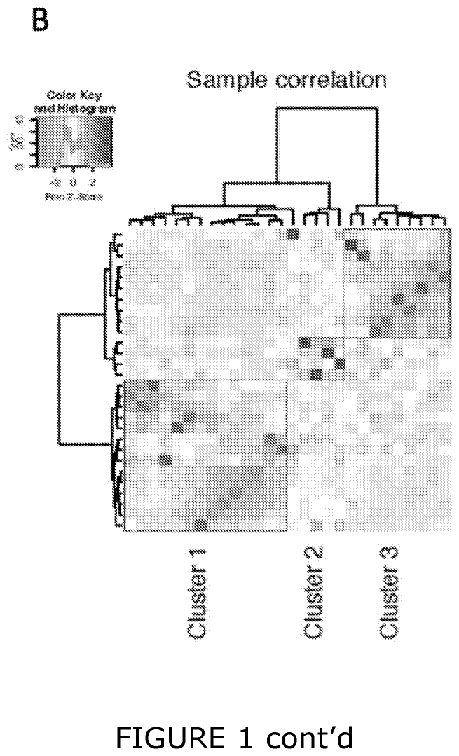

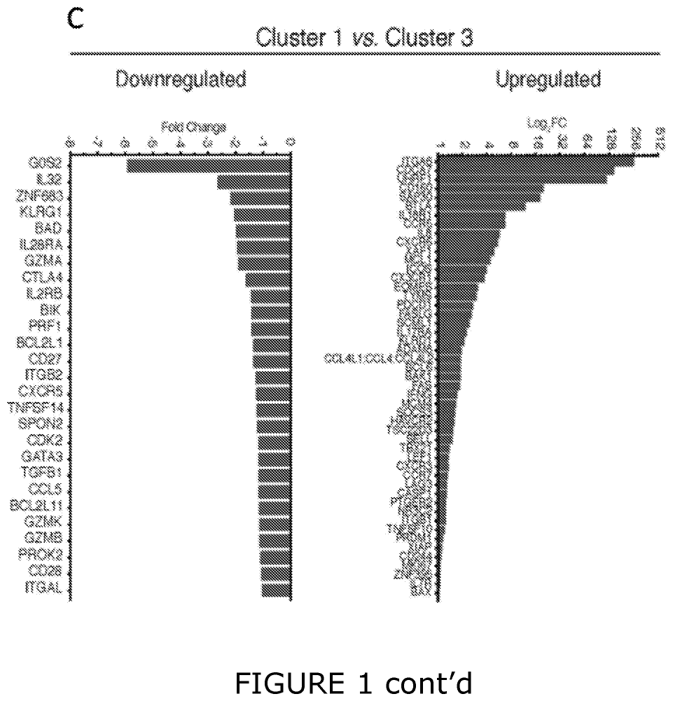

The present inventors have previously demonstrated that cytomegalovirus (CMV)-specific T-cells can be characterized based upon differential expression of key transcription factors (T-bet and Eomes) and effector molecules (perforin and granzymes). In order to broadly characterize the phenotypic and functional properties of these populations a customized qPCR array was established, targeting a panel of 95 immune related genes selected for their role in CD8⁺ T-cell function and differentiation. Gene expression was assessed in 27 distinct CMV-specific CD8⁺ T-cell populations sorted using MHC-Multimer staining. Clustering analysis enabled further definition of the differential phenotypic profiles. Consistent with their previous findings, the present inventors identified two predominant clusters that displayed distinct expression of key effector molecules, including GzmB, A and K; and the transcriptional regulator ZNF683, which encodes the Hobit protein (FIG. 1A). Strikingly, a key driver of these population clusters was the differential expression of the ITGA6 transcript that encodes CD49f and displayed over 200-fold higher expression in Cluster 1. To validate these observations, the expression of CD49f was assessed in CMV-specific T-cells. Samples were gated on viable CD8⁺ lymphocytes and assessed for CD49f-expression in CMV MHC-Multimer⁺ cells (FIG. 1B). Variable CD49f expression was observed in CMV-specific CD8⁺ T-cells from different volunteers who were characterized as high, intermediate or low.

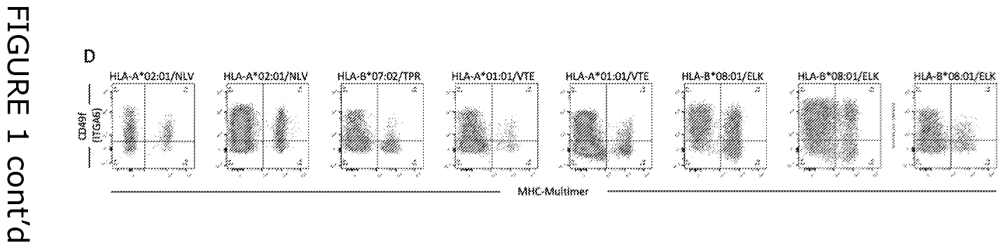

These initial observations in CMV-specific T-cells suggested that CD49f may provide a novel marker for defining populations of memory T-cells in humans. The present inventors therefore explored its co-expression with a panel of T-cell differentiation markers including CD95, CD27, CD28, CCR7, CD45RA and CD57. High CD49f expression correlated with the expression of CD27 and CD28 in memory T-cells relative to naïve T-cells (FIG. 2A). Memory phenotype analysis demonstrated that CD49f expression peaked in CD27⁺ CD28⁺ memory T-cells (FIG. 2B), with the majority of T-cells expressing high levels of CD49f restricted to the population (FIG. 2C), whereas, T-cells expressing intermediate levels of CD49f could be found in all memory populations.

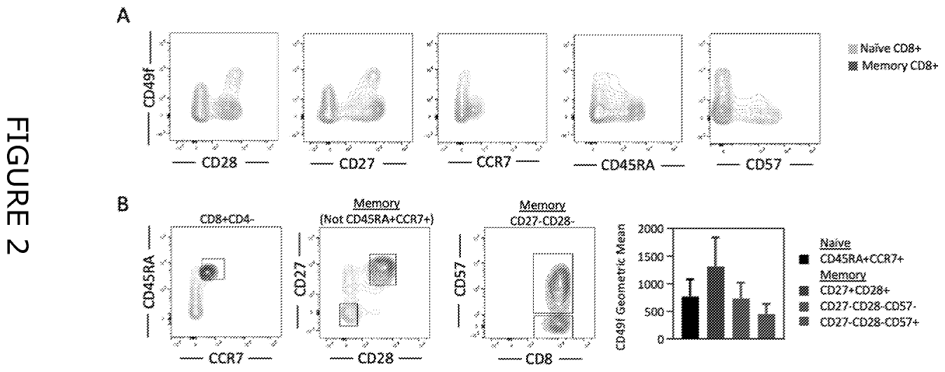

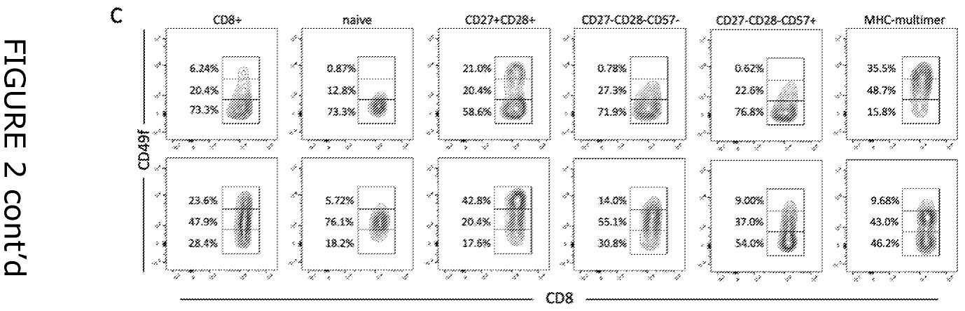

The present inventors next explored the co-expression of CD49f with other markers of T-cell differentiation including key transcriptional regulators of effector function and T-cell differentiation. While CD49f^{lo} and CD49f^{int} cells were enriched for co-expression of T-bet, Eomes and Hobit, which was coincident with higher expression of the effector molecular granzyme B, CD49f^{hi} cells, typically displayed low levels of these molecules (FIG. 3A). CD49f^{hi} cells also displayed evidence of differential expression of other integrin molecules, with evidence for higher expression of CD29, which pairs with CD49f to produce integrin α6β1, whilst they displayed lower expression of CD11a and CD18, which pair to form Lymphocyte function-associated antigen 1. These observations are consistent with a less differentiated central memory phenotype evident in CD49f^{hi} cells.

To further explore the relationship of CD49f with a less differentiated phenotype, the present inventors assessed co-expression of CD49f with the transcriptional regulators, T-cell factor 1 (TCF-1) and lymphoid enhancer-binding factor 1 (LEF-1). LEF-1 and TCF-1 are key regulators of cellular differentiation in T-cells and were recently shown to be expressed in self-renewing human CD8⁺ T-cells and can be maintained following proliferation. Both LEF-1 and TCF-1 expression correlated with high expression of CD49f in memory T-cells (FIG. 3B). Only CD49f high cells displayed expression levels similar to that seen in naïve cells. Cells expressing intermediate levels of CD49f maintained a high proportion of TCF1^{hi} cells, whilst lack of CD49f expression was more consistent with low TCF1 and LEF1 expression. To assess the impact of CD49f expression on the proliferative potential of memory CD8⁺ T-cells, the present inventors sorted memory T-cells expressing high, intermediate and low levels of CD49f, stimulated with anti-CD3 and ant-CD28 coated beads, then assessed proliferation and TF expression. Expression of both TCF1 and LEF1 expression was maintained in a significant proportion of CD49f^{hi} cells following proliferation (FIG. 3C)

### Virus-Specific Human CD8⁺ T-Cells Immune Reconstitution and Expression of CD49f

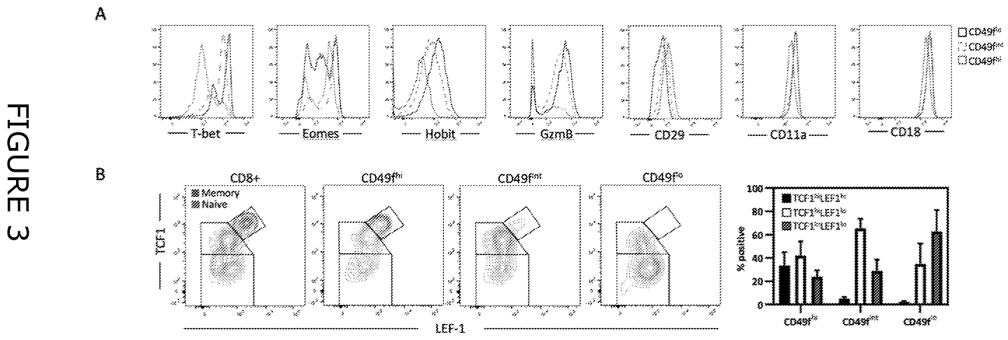

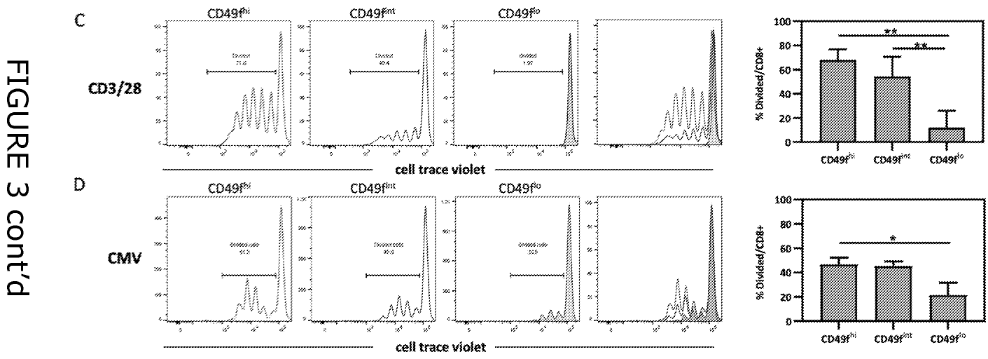

Efficient immune reconstitution following hematopoietic stem cell transplant (HSCT) is critical for the maintenance of immunity against CMV and many other human pathogens. The present inventors therefore sought to explore the differential expression of CD49f following immune reconstitution in ten patients who had received a HSCT. CD8⁺ T-cells were assessed for the expression of CD49f at one month and three months following HSCT. At one-month post-transplant, the global CD8⁺ T-cell population was dominated by CD49f^{int} and CD49f^{hi} T-cells, with relatively few cells expressing a CD49f^{lo} phenotype (FIGS. 4A&B). However by three months, while there was little change in the proportion of CD49f^{hi} cells, the majority of recipients shown a dramatic increase in the proportion of CD49f^{lo} cells, which is consistent with previously observed expansion of CMV-specific effector populations in this cohort of HSCT recipients. Analysis of CMV-specific MHC-multimer⁺ cells, demonstrated a similar expansion in CD49f^{lo} CMV-specific T-cells by three months post-transplant (FIGS. 4C&D). However, it was also noted that the majority of patients showed an increase in CD49f^{hi} CMV-specific T-cells at 3 months, indicative of the establishment of a distinct memory population within the CMV-specific compartment that is likely critical for self-renewal and the long-term maintenance of CMV immunity.

While a large proportion of patients was observed who displayed an expansion of CD49f^{lo} cells by 3 months, a proportion, including patient 26, did not. The HSCT recipients included in this study have previously been characterized for risk of CMV-reactivation/disease post-transplant, which was defined by their capacity to generate stable CMV-specific T-cell immunity. It was therefore sought to explore differential CD49f expression in recipients with stable and unstable CMV-immunity. While there were no significant differences in the proportion of CD49f^{hi} T-cells at 1 or 3 months post-transplant, at 3 months post-transplant patients with stable immunity had a significantly higher proportion CD49f^{lo} CD8⁺ T-cells (FIG. 4E). This was associated with a significantly reduced peak CMV viremia in these receipts in the first three months post-transplant (FIG. 4F). In addition to patients who developed CMV viremia during the acute stages post-transplant, the present inventors also had access to two patients with late-stage CMV-complications that were associated with symptomatic disease. While both of these patients showed evidence of a high frequency of effector CD49f^{lo} CD8⁺ T-cells early post-transplant, this population declined over time in both patients (FIG. 4G), indicative of the loss of CMV immune control in these chronically infected individuals over time.

### Impact of CD49f Expression on Potential Efficacy of Adoptive Cellular Immunotherapy

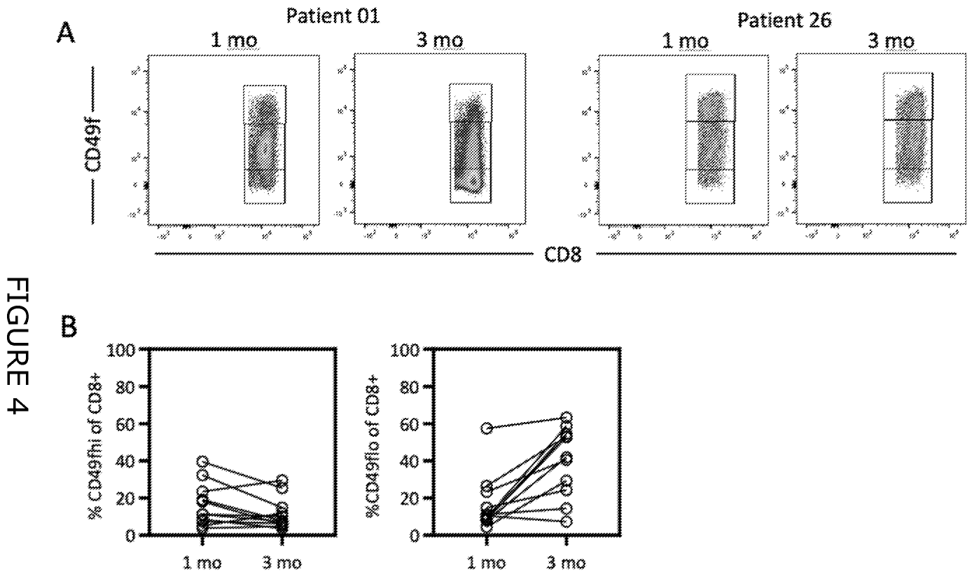

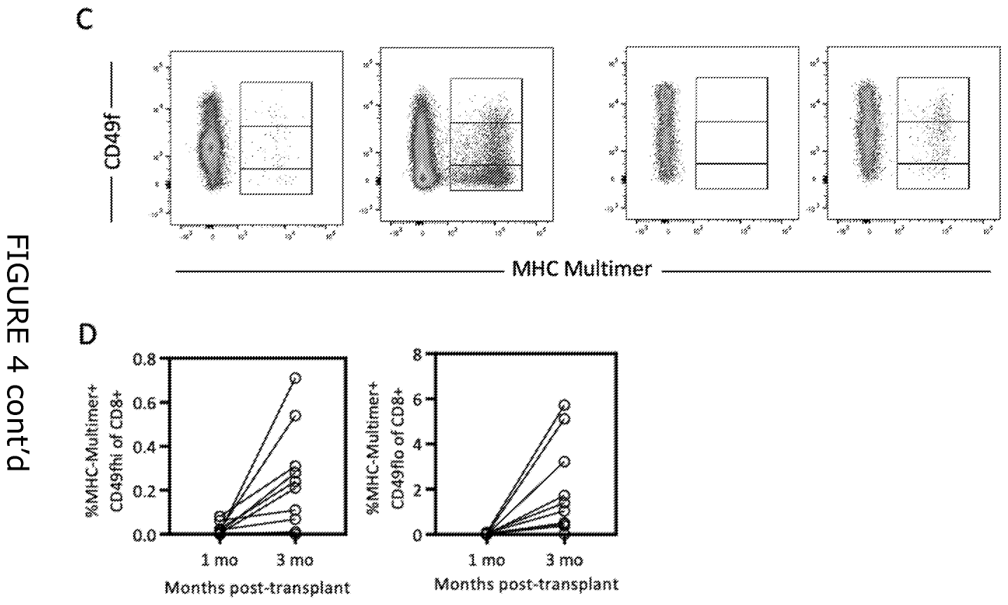

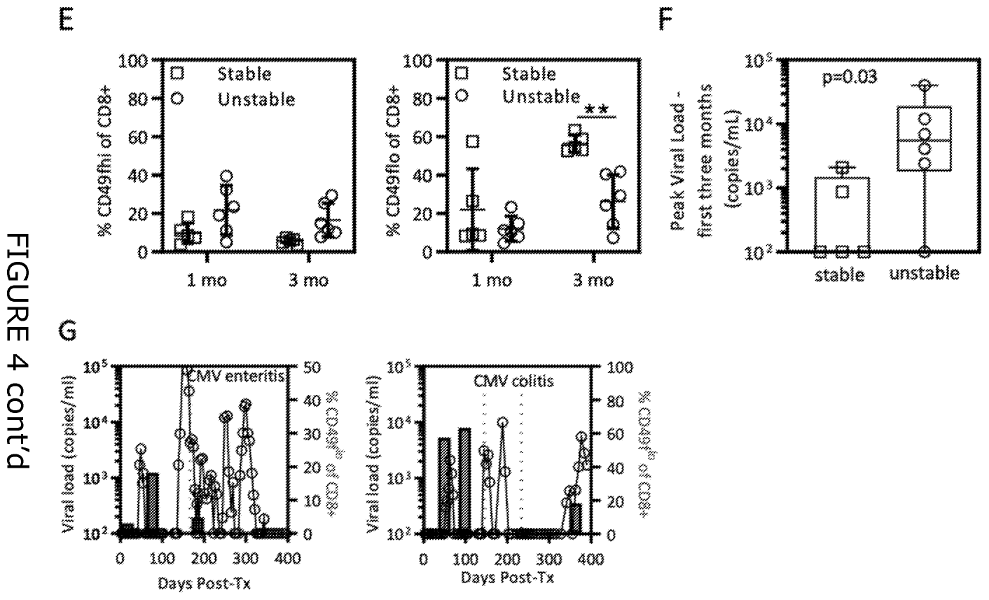

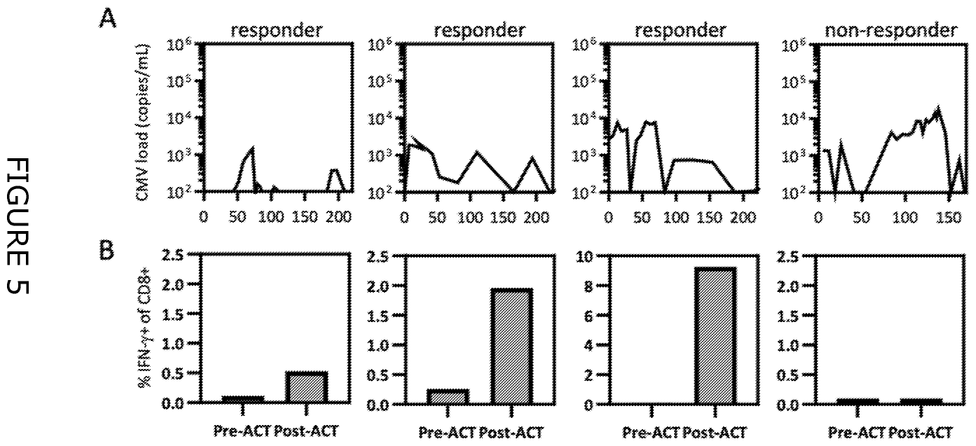

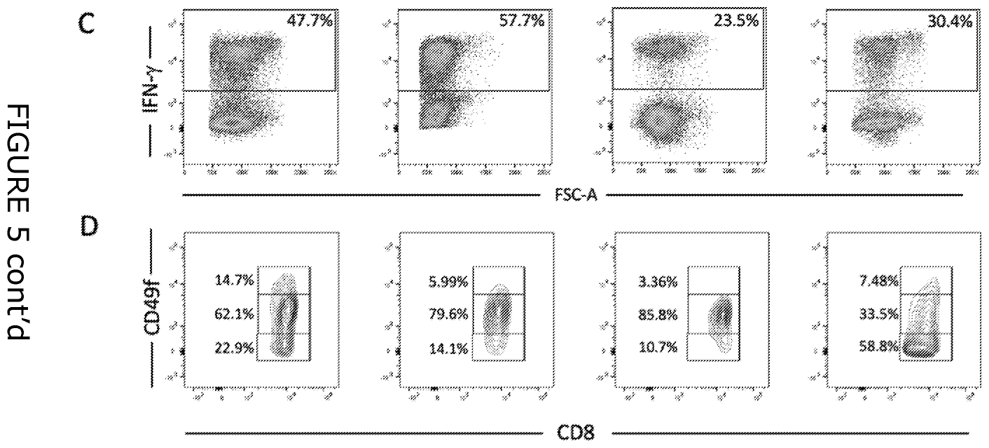

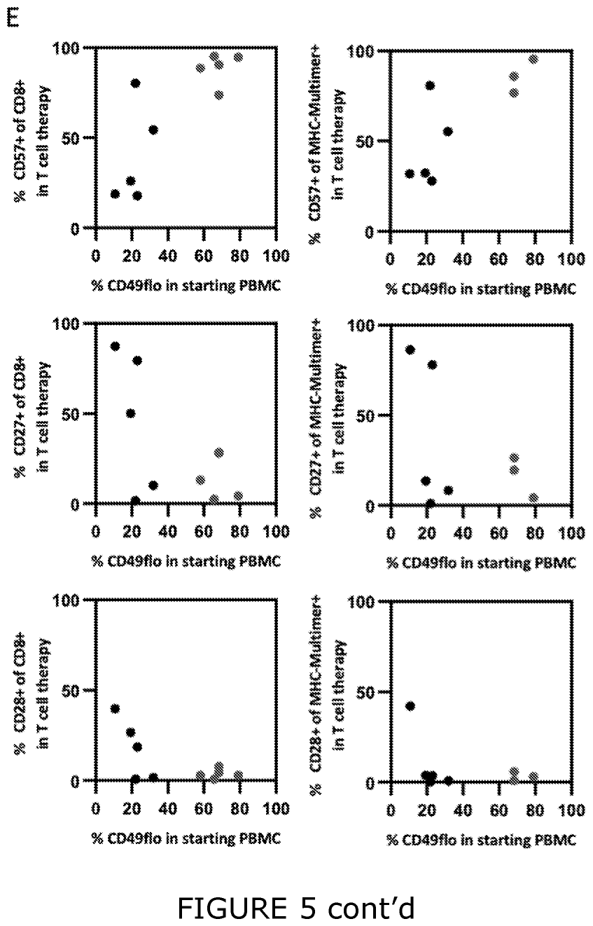

The present inventors recently reported on the successful use of adoptive cell therapy (ACT) to treat CMV-associated complications in solid organ transplant (SOT) recipients. In this light, they sought to explore if differences in CD49f expression were associated with response to therapy. Four ACT recipients were selected for whom material was available. Three of these patients displayed evidence of immune-mediated control of CMV following ACT, characterized by a reduction in CMV viremia in the peripheral blood (FIG. 5A) and an increase in CMV-specific T-cell immunity following ACT (FIG. 5B), whereas the final patients shown no evidence of T-cell mediated immune control following ACT. Notably, this analysis revealed that whilst all of the patients showed strong reactivity to CMV in their cellular product (FIG. 5C), analysis of the PBMC used to generate the therapeutic product indicated that the non-responding patient contained a high proportion of CD49f^{lo} T-cells prior to cell expansion (FIG. 5D), despite the lack of CMV-specific immunity pre-ACT. The responding patients who also displayed low CMV immunity pre-ACT, had a much lower proportion of CD49f^{lo} cells prior to expansion. This patient was previously reported as showing dramatically reduced clonal diversity in their cellular product.

### CD49f Expressing T-Cells Retain Increased Proliferative Potential after In Vitro Expansion

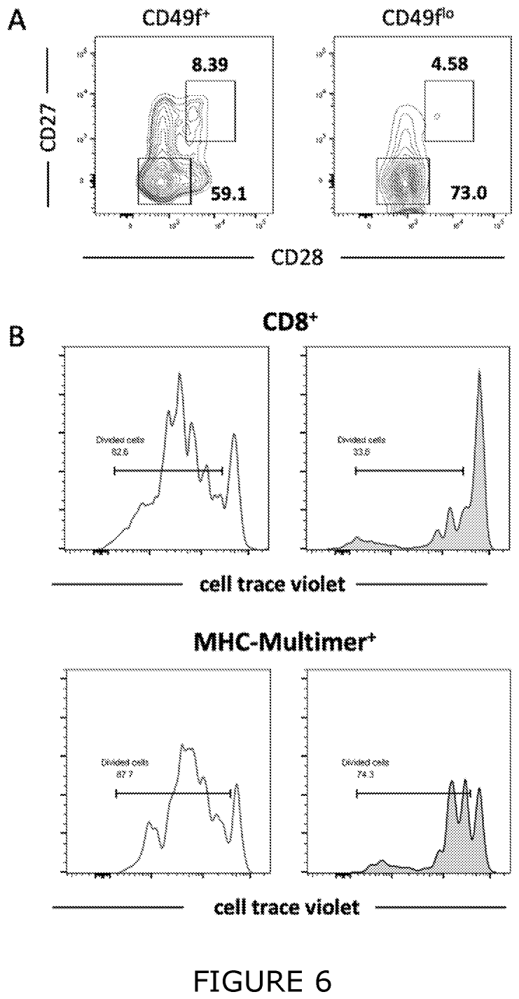

The observations in the SOT patients suggested that the retention of CD49f expression in precursor memory T-cell populations could promote better outcomes in patients treated with ACT by promoting better survival and proliferation following cell transfer. To assess this, a protocol was developed to magnetically separate CD49f⁺ PBMC. PBMC from a CMV-seropositive healthy volunteer were sorted into CD49f⁺ and CD49f^{lo} populations then stimulated with irradiated autologous PBMC pulsed with a pool of HLA-defined CMV peptide epitopes. Cells were cultured for 14 days in the presence of interleukin-2 (IL-2) then assessed for the expression of the central memory markers, CD27 and CD28. T-cells generated from the CD49f⁺ population retained a higher proportion of CD27⁺ CD28⁺ T-cells (FIG. 6A), consistent with the observations in SOT patients. To assess the retention of proliferative potential following stimulation, cultured cells were labelled with cell trace violet, then stimulated again with the CMV-specific peptide pool. Both CD8⁺ and MHC-multimer specific T-cells displayed greater proliferative potential in cultures generated from the CD49f⁺ populations, reiterating our observations in SOT patients.

### T Cells Generated from the CD49f⁺ Compartment Show Improved Efficacy in a Humanized Model of Epstein Barr Virus Associated Lymphoma

PBMC were magnetically sorted into CD49f⁺ and CD49f. populations, then stimulated with EBV-encoded peptide epitopes pulsed onto autologous PBMC. T-cells were cultured in the presence of IL-2 for 17 days, assessed for EBV-reactivity then cryopreserved.

Immunodeficient mice were injected subcutaneously with EBV-transformed B cells HLA matched to the CD49f⁺ and CD49f⁻ T-cells. Mice were assessed for tumor formation, then after 16 days six mice per group were injected intravenously with 5 million T-cells generated from either the CD49f⁺ or CD49f⁻ compartment. One day later mice were injected with anti-PD1 antibody. On day 20 and 21, mice were treated with a second dose of T-cells and anti-PD1 respectively. Mock mice received a mock injection of PBS and control IgG4. Mice were monitored for tumor growth until day 31.

While EBV-specific T-cells generated from both the CD49f⁺ and CD49f⁻ compartment controlled tumor growth, this effect was more pronounced in the CD49f⁺ T-cell treated mice with a median tumor size of 14.5 mm² on Day 31, compared to 43.0 mm² for the mice treated with the CD49f⁻ T-cells and 137.5 mm² for the mock treated mice (FIG. 7).

These observations demonstrate the potential of T-cells generated from the CD49f⁺ compartment to improve efficacy in an adoptive therapeutic setting.

### Association of LEF1, TCF1 and CD49f (ITGA6)

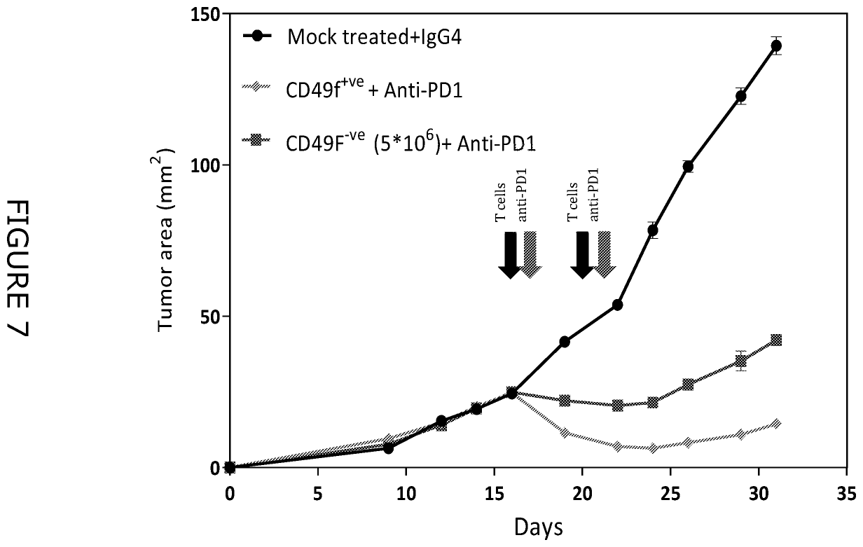

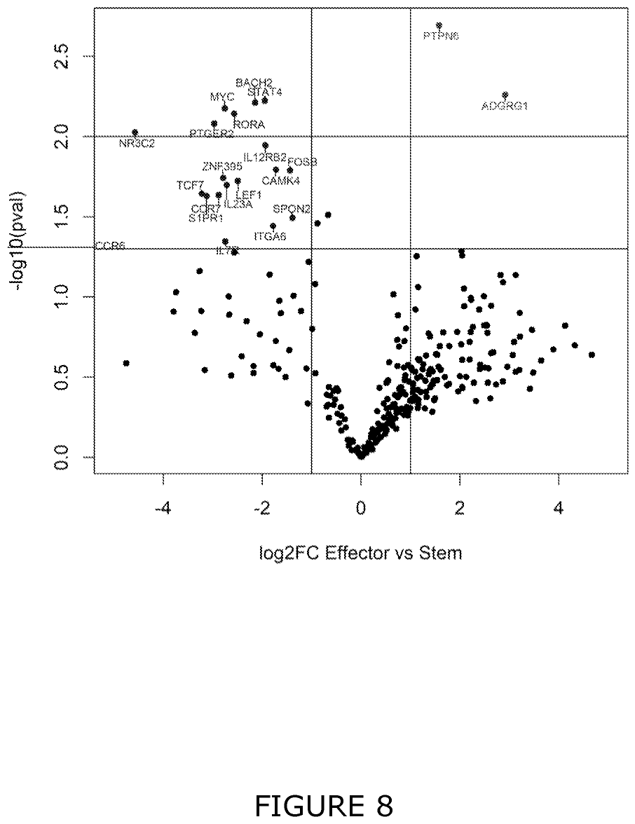

Using publicly available data the present inventors examined the gene expression profile of tumour infiltrating cells (TILs) sorted from prostate and bladder cancer tumours (Jansen et al., 2019. PMID: 31827286). This study identified two distinct populations within the TILs that were associated with either terminally differentiated or stem-like phenotypes. Using the RNAseq dataset from this study we identified that expression of the genes LEF1, TCF7, and ITGA6 (CD49f) is upregulated in this stem-like population of TILs (FIG. 8). These data suggest that CD49f expression has an important role in the maintenance of stem-like qualities in tumour infiltrating T-cells.

### Differential Gene Expression in CD8⁺ T Cells Defined by CD49f Expression Levels

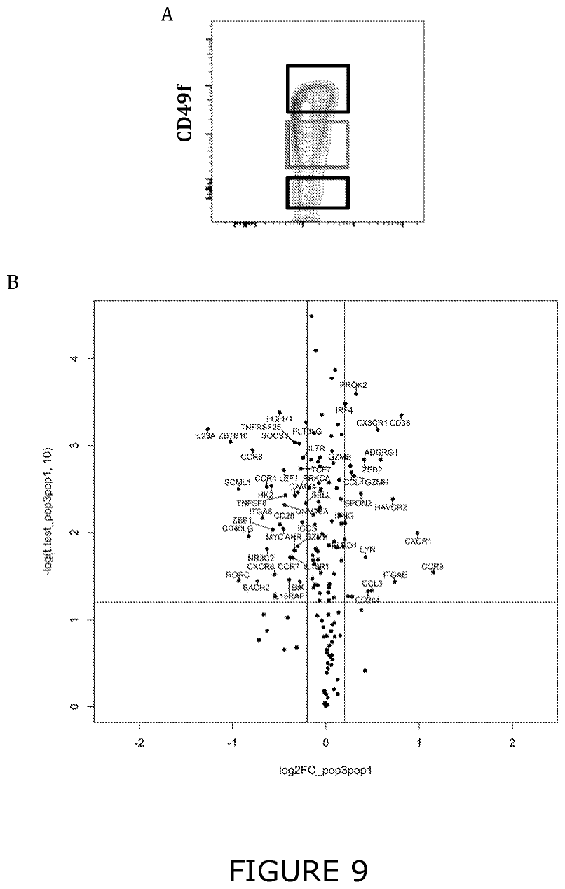

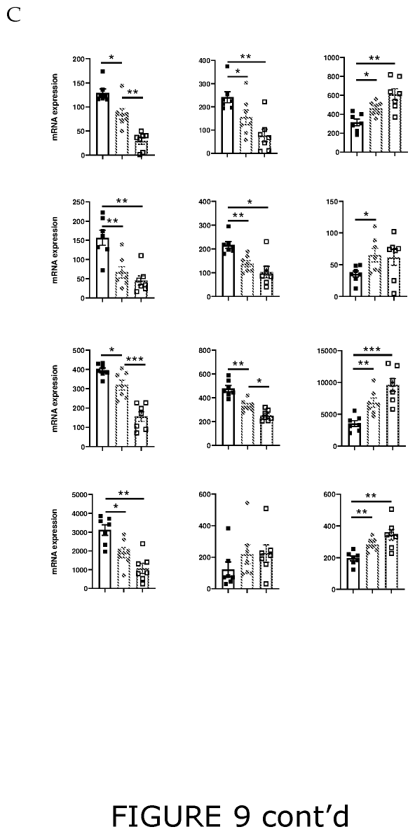

These observations suggested that the differential expression of CD49f may define functionally unique subsets of human memory CD8⁺ T-cells. To define the attributes of these T-cells, memory CD8⁺ T-cells from six volunteers were sorted based upon their level of CD49f expression (low, intermediate and high). We then assessed gene expression using the NanoString nCounter gene expression platform on an enlarged custom set of 326 T-cell associated genes. A heat map of differential gene expression in CD49f low (CD49f^{lo}), CD49f intermediate (CD49f^{int}) and CD49f high (CD49f^{hi}) expressing CD8⁺ T-cells is shown in FIG. 9A. As expected, the degree of CD49f (ITGA6) expression coincided with significant differences in the expression of multiple memory T-cell markers, suggesting that CD49f surface expression could be used to identify distinct subpopulations of CD8⁺ memory T-cells. High CD49f expression was associated with significantly increased expression of the central memory marker CD28 (FIG. 9B) but significantly reduced expression of effector memory markers including TBX21, EOMES and NKG7 (FIG. 2B). In addition, significant differences in the expression of transcriptional regulators of T-cell function, including LEF1 and T-cell factor 7 (TCF7) were observed in CD49f^{hi} versus CD49f^{lo} CD8⁺ T-cells (FIG. 2B). Interestingly, CD49f intermediate cells (CD49f^{int}) displayed a gene expression profile with intermediate characteristics of both the CD49f^{hi} and CD49f^{lo} cells (FIG. 9B). Despite significantly reduced LEF1 expression, in comparison to their CD49f^{hi} counterparts, there was evidence that these CD49f^{int} CD8⁺ T-cells retain TCF7 expression while significantly increasing expression of effector genes including IFNG and TBX21 (FIG. 9C).

### Efficacy of CAR19-T-Cells Generated from the CD49f^{hi} Compartment

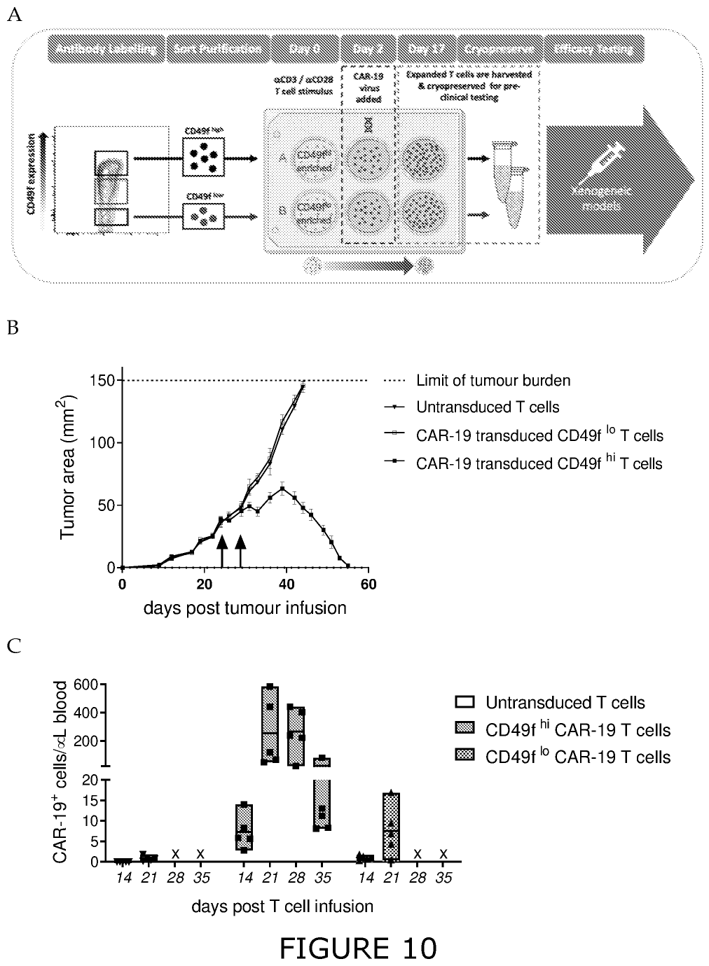

Given the observed association between CD49f expression, the self-renewal markers LEF1 and TCF7, and the corresponding increase in proliferative potential, the present inventors next sought to assess whether this CD49f compartment could be utilized to generate an adoptive cell therapy (ACT) with enhanced efficacy. To establish a robust method for generating ACT from the CD49f⁺ compartment CD49f^{hi} cells and CD49f^{lo} cell fractions were purified using anti-CD49f antibodies and fluorescence activated cell sorting (FACS). From here the CD49f^{hi} and CD49f^{lo} populations were cultured together with anti-CD3/anti-CD28 beads in order to stimulate T-cells in vitro (FIG. 10A). On day 2 of culture the T-cells were transduced with a CAR19 lentiviral construct and expanded in culture media supplemented with IL-2. On culture day 17 the expanded T-cells were harvested, the rate of CAR19 transduction efficiency was assessed by ICS, and the T-cells were cryopreserved.

To assess the therapeutic potency of these adoptive cell therapy (ACT) products, they were challenged in an EBV⁻ Burkitt lymphoma cell line (BJAB)-derived xenogeneic tumour model. Here, eight week old NSG female mice were injected subcutaneously with BJAB-cells and, after tumours reached 25 mm² in size, experimental groups were treated with two doses (at an interval of 96 hours) of expanded T-cells generated from either CD49f^{hi} or CD49^{lo} sort-enriched cells or with untransduced T cells as a biological control. Xenogenic experimental groups were monitored weekly to assess tumour size (FIG. 10B) and peripheral blood monitored to assess the in vivo expansion of CAR19-T cells (FIG. 10C).

ACT generated from the CD49f^{hi} (black square) T-cells displayed significantly enhanced tumour control compared to both CD49f^{lo} (open square) and untransduced (black triangle) T-cell treated groups (FIG. 10B). Peripheral blood monitoring revealed that at day 21 post treatment, human (CD45⁺) CAR19⁺ T-cells displayed greater expansion in the CD49f^{hi}-derived T-cell group in comparison to their CD49f^{lo}-derived counterparts (FIG. 10C). This increased expansion in CD49f^{hi} CAR19⁺ T-cells is associated with BJAB tumour clearance, which was not observed in experimental groups treated with either CD49f^{lo} or untransduced T cells (FIG. 10B).

### Materials & Methods

Ethics

Healthy volunteers and transplant recipients were recruited according to according to the principles of the Declaration of Helsinki and the National Statement on Ethical Conduct in Human Research in accordance with the National Health and Medical Research Council (Australia) Act. The Human Ethics Committees of the QIMR Berghofer Medical Research Institute and Royal Brisbane and Women's Hospital approved the study protocol for the recruitment of HSCT patients. Solid-organ transplant recipients treated with adoptive immunotherapy have been previously described (Smith et al., 2018). This study was approved by the QIMR Berghofer Medical Research Institute Human Research Ethics Committee, The Prince Charles Hospital Human Research Ethics Committee and the Royal Adelaide Hospital Research Ethics Committee, and registered under the Australian New Zealand Clinical Trial Registry (ACTRN12613000981729). The recruitment of healthy volunteers was approved by The Human Ethics Committees of the QIMR Berghofer Medical Research Institute.

MHC Multimers

MHC-peptide dextramers supplied by Immundex, or MHC peptide tetramers made in-house, were used to detect epitope-specific CD8⁺ T-cells. PBMC were incubated with either allophycocyanin (APC), phycoerythrin (PE) or brilliant violet (BV) 421 labelled with MHC class I multimers specific for the CMV specific peptide epitopes listed in table 1, then assessed for the cell phenotype and function or for gene expression following cell sorting as outlined below.

TABLE 1

| | | HLA |
|---|---|---|
| Epitope | Code | Restriction |
| NLVPMVATV | NLV | HLA-A*02:01 |
| VTEHDTLLY | VTE | HLA-A*01:01 |
| RPHERNGFTVL | RPH | HLA-B*07:02 |
| TPRVTGGGAM | TPR | HLA-B*07:02 |
| ELRRKMMYM | ELR | HLA-B*08:01 |
| ELKRKMIYM | ELK | HLA-B*08:01 |

Analysis of Gene Expression Using a TaqMan Gene Array Card

The TaqMan gene array card used to assess gene expression in CMV-specific T-cells has been described (Schuessler et al., 2014). Briefly, PBMC were labeled with MHC-multimers as outlined above, then stained for anti-CD4 and anti-CD8. CD8⁺ MHC-multimer⁺ cells were then sorted using a BD FACSAria. Total RNA was purified from all sorted T-cells using the Qiagen RNeasy Micro kit according to the manufacturer's instructions and eluted with a final volume of 12 μL. Then, a high capacity RNA to cDNA kit (Life Technologies) was used to transcribe a volume of RNA equivalent to 3000 cells into cDNA. Following a 14-cycle pre-amplification step, the cDNA was then loaded into the custom designed TaqMan array cards and the PCR performed using the Viia7 (Life Technologies). Three housekeeping genes, 18S, B2-microglobulin and actin where used to normalise gene expression data. Expression analysis was performed using Gene Spring software.

Flow Cytometric Analysis of CD49f Expression in CD8⁺ T-Cells

PBMC from CMV-seropositive were incubated with MHC class I multimers, followed by anti-CD8 (V500 or perCPCy5.5), anti-CD4 (PeCy7), Live/Dead Near IR and anti-CD49f BV421. For phenotypic analysis cells were co-incubated with anti-CD27 (PE-Dazzle), anti-CD28 (BV480), anti-CD45RA (FITC), anti-CD57 (BV605), anti-CCR7. For integrin analysis cells were co-incubated with anti-CD29, anti-CD11a and ant-CD18. Cells were fixed and permeabilized using BD TF Fixation/Permeabilization Solution. Cells were then was with Perm/Wash and incubated with anti-Hobit (Vieria Braga et al., 2015) followed by PE-conjugated anti-mouse IgM, or with anti-T bet (PE), anti-Eomes (perCP-efluor710) and anti-Granzyme B (AF700). Cell acquisition was performed using a BD LSR Fortessa and post-acquisition analysis performed using FlowJo software.

Cell Trace Violet Proliferation Assay

To assess proliferation of T-cells from PBMC, cells were labeled with cell trace violet, then stained for anti-CD4, anti-CD8 Live/Dead Near IR and anti-CD49f as outlined above. CD8⁺ T-cells were then sorted into CD49f^{hi}, CD49f^{int} and CD49f^{lo} populations and stimulated days with anti-CD3/anti-CD28 beads. Four days later cells were stained for anti-CD4, anti-CD8 and Live/Dead Near IR and assessed for proliferation using a BD LSR Fortessa. Post-acquisition analysis performed using FlowJo software. To assess the recall proliferative response of cultured T-cells, PBMC were sorted labeled with biotinylated anti-CD49f, then bound to anti-biotin microbeads from Miltenyi Biotech. PBMC were then sorted into CD49f⁺ and CD49f^{lo} populations using MS columns from Miltenyi Biotech. Cells were stimulated with autologous irradiated PBMC pulsed with a pool of defined CMV-specific peptide epitopes and cultured for 14 days in the presence of interleukin 2. On Day 14, cells were labelled with cell trace violet and assessed for proliferation as outlined above following recall with the CMV-specific peptide pool.

Intracellular Cytokine Analysis

PBMC were stimulated with a pool of CMV-encoded T-cell epitopes as described (ref). Cells were acquired using a BD LSR Fortessa with FACSDiva software (BD Biosciences) and post-acquisition analysis was performed using FlowJo software (TreeStar).

Flow Cytometric Analysis of CD49f Expression in CD8⁺ T Cells

PBMC were incubated for 30 minutes at 4° C. with the following antibodies: anti-CD8 (perCPCy5.5), anti-CD4 (PeCy7), Live/Dead Near IR and anti-CD49f (BV421). Following staining, cells were washed with PBS containing 2% FCS and fixed using BD Fixation Solution (BD Biosciences). All antibodies were sourced from either Biolegend or BD Biosciences. Cell acquisition was performed using a BD LSR Fortessa and post-acquisition analysis performed using FlowJo software.

Analysis of Gene Expression Using NanoString.

Using the BD Aria III flow cytometer, CD8⁺ T-cells were sort-purified based on their CD49f expression levels into: CD49f^{hi}, CD49f^{int} and CD49f^{lo} populations. Total RNA was purified from all sorted T-cells using the Qiagen RNeasy Micro kit according to the manufacturer's instructions and eluted with a final volume of 15 μL. Gene expression was assessed using the NanoString nCounter® gene expression platform on an enlarged custom set of 326 T-cell associated genes. Expression analysis was performed using nSolver™ Analysis Software.

Xenogeneic Mouse Models

The EBV-negative, Burkitt lymphoma malignant human B-cell line (BJAB) tumour cells were expanded in vitro in RPMI 1640 (Gibco) and injected subcutaneously into eight week old NSG female mice. Tumour size was monitored weekly and once tumours reached 25 mm² in size, experimental groups were treated with two doses (at a 96 hour interval) of expanded T cells. Xenogenic experimental groups were monitored weekly to assess tumour size and animals were sacrificed once tumours reached a size of 150 mm². Peripheral blood was monitored weekly from day 7 post T-cell treatment, to assess the in vivo expansion of the huCD45⁺ T-cell compartment and to identify CAR19⁺ T-cells.

Peripheral Blood Monitoring

Peripheral blood was obtained from experimental mice on days +7, +14, +21, +28 and +35 (where survival permits). Blood was incubated with antibodies for 30 minutes at 4° C. with the following antibody master mix: mouse anti-CD45 (V450), and human anti-CD45 (V500), anti-CD3 (APC), anti-CD8 (perCPCy5.5), anti-CD4 (AF700) and Live/Dead (Near IR). After staining 350 μL of FACS Lyse solution (BD Biosciences) was added to the blood stain as per the manufacturers protocol and incubated at room temperature for a further 15 minutes. Precision Count Beads added were vortexed thoroughly and 20 μL of beads added to the stained blood preparation. All antibodies were sourced from either Biolegend or BD Biosciences. CAR19⁺ T cells were identified by endogenous expression of red fluorescent protein (RFP). Cell acquisition was performed using a BD LSR Fortessa and post-acquisition analysis performed using FlowJo software.

Statistical Analysis

GraphPad Prism 8.2.1 (San Diego, Calif., USA) was used to perform statistical analysis. Statistical comparisons between groups were made using unpaired Mann-Whitney U tests. Bar graphs represent individual samples with each group displayed as mean with SEM. P<0.05 was considered statistically significant.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

### REFERENCES

Schuessler, A., Smith, C., Beagley, L., Boyle, G. M., Rehan, S., Matthews, K., Jones, L., Crough, T., Dasari, V., Klein, K., Smalley, A., Alexander, H., Walker, D. G., Khanna, R. Autologous T-cell therapy for cytomegalovirus as a consolidative treatment for recurrent glioblastoma. 2014, Cancer Res, 74(13): 3466-76.
Smith C, Beagley L, Rehan S, Neller M A, Crooks P, Solomon M, Holmes-Liew C L, Holmes M, McKenzie S, Hopkins P, Campbell S, Francis R, Chambers D, Khanna R. Autologous adoptive T-cell therapy for recurrent or drug-resistant cytomegalovirus complications in solid organ transplant patients: A single-arm open-label phase I clinical trial. Clinical Infectious Diseases. 2018 Jul. 5. doi: 10.1093/cid/ciy549.
Vieira Braga, F. A., Hertoghs, K. M. L., Kragten, N. A. M., Doody, G. M., Barnes, N. A., Remmerswaal, E. B. M., Hsiao, C. C., Moerland, P. D., Wouters, D., Derks, I. A. M., van Stijn, A., Demkes, M., Hamann, J., Eldering, E., Nolte, M. A., Tooze, R. M., ten Berge, I. J. M., van Gisbergen, K. P. J. M., van Lier, R. A. W. Blimp-1 homolog Hobit identifies effector-type lymphocytes in humans. 2015, Eur J Immunol. 45 (10): 2945-58.

What is claimed is:

1. An isolated T-cell population that comprises CD49f⁺ T-cells wherein the CD49f⁺ T-cells constitute at least 1% (including at least 2% to 99% and all integer percentages therebetween) of the T-cells in the population

2. The isolated population of claim 1, wherein the CD49f⁺ T-cells have one or more immune properties selected from an early memory phenotype, a stem-like phenotype, increased proliferative potential, increased survival and increased persistence in vivo, decreased differentiation, increased immune effector function, decreased immune effector dysfunction and increased responsiveness in immunotherapy.

3. The isolated population of claim 1 or claim 2, wherein the CD49f⁺ T-cells comprise CD49f^{hi} T-cells, CD49f^{int} T-cells, or both.

4. The isolated population of any one of claims 1 to 3, wherein the CD49f⁺ T-cells comprise memory T-cells (e.g., central memory T-cells) such as, but not limited to, CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T- cells, CD49f⁺ CD27⁺ CD28⁺ CD95⁺ memory T-cells, CD49f⁺ CD27⁺ CD28⁺ CD45RA⁺ CD95⁺ memory T-cells, CD49f⁺ CD27⁺ CD28⁺ CD95⁺ CCR7⁺ memory T-cells and CD49f⁺ CD27⁺ CD28⁺ CD45RA⁺ CD95⁺ CCR7⁺ memory T-cells, wherein the memory cells are optionally positive for CD127.

5. The isolated population of any one of claims 1 to 4, wherein the CD49f⁺ T-cells are positive for one or both of CD4 and CD8.

6. The isolated population of any one of claims 1 to 5, wherein the CD49f⁺ T-cells are positive for TCF-1 (e.g., TCF-1^{hi}) and/or LEF-1 (e.g., LEF-1^{hi}) and optionally positive for one or both of Oct4 and Sox2.

7. The isolated population of any one of claims 1 to 6, wherein the CD49f⁺ T-cells in the isolated population constitute 1% or more of the T-cells in the population, including 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or up to and including 100% of the T-cells in the isolated population.

8. The isolated population of any one of claims 1 to 6, wherein the CD49f⁺ T-cells in the isolated population constitute 10% or more of the total number of cells in the population, including 1% or more of the T-cells in the population, including 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or up to and including 100% of the total number of cells in the isolated population.

9. The isolated population of any one of claims 1 to 8, wherein the isolated population is a substantially homogeneous population.

10. The isolated population of any one of claims 1 to 9, wherein the CD49f⁺ T-cells express a recombinant T-cell receptor (rTCR).

11. The isolated population of any one of claims 1 to 9, wherein the CD49f⁺ T-cells express a chimeric antigen receptor (CAR), wherein the CAR or CAR-expressing T-cell is suitably selected from a T-cell Redirected for Universal Cytokine Killing (“TRUCK”), Universal CAR, Self-driving CAR, Armored CAR, Self-destruct CAR, Conditional CAR, Marked CAR, TenCAR, Dual CAR, or safety CAR.

12. The isolated population of claim 11, wherein the CAR targets CD19.

13. The isolated population of claim 11, wherein the CAR targets any one of the group comprising: CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signalling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumour-associated glycoprotein (TAG)-72, CD38, CD44v6, carcinoembryonic antigen (CEA), epithelial cell adhesion molecule (EpCAM), KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGS5), high molecular weight melanoma-associated antigen (HMWMAA), o-acetyl-GD2 ganglioside, folate receptor beta, TEM1/CD248, tumour endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor .gamma.-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAX5, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), signalling lymphocyte activation molecule (SLAM) family member 6 (SLAMF6), SLAMF4, or any combination thereof.

14. A process of manufacturing a T-cell population comprising T-cells with enhanced immune properties (e.g., selected from one or more of an early memory phenotype, a stem-like phenotype, increased proliferative potential, increased survival and increased persistence in vivo, decreased differentiation, increased immune effector function, decreased immune effector dysfunction and increased responsiveness in immunotherapy), the process comprising or consisting essentially of: isolating or selecting from a sample containing T-cells a T-cell population comprising CD49f⁺ T-cells, wherein the CD49f⁺ T-cells constitute at least 1% (including at least 2% to at least 99% and all integer percentages therebetween) of the T-cells in the population, or enriching a sample containing T-cells for CD49f⁺ T-cells, thereby manufacturing a T-cell population comprising T-cells with enhanced immune properties.

15. The process of claim 14, further comprising harvesting the T-cell-containing sample from a suitable source.

16. The process of claim 15, wherein the source is selected from a peripheral blood mononuclear cell (PBMC) sample, cord blood cells, a purified population of T-cells, a T-cell line, or a sample obtained by leukapheresis.

17. The process of any one of claims 14 to 16, wherein T-cell-containing sample is enriched for T-cells of interest, for example CD8⁺ T-cells, CD4⁺ T-cells, naïve T-cells, memory T-cells, previously activated T-cells and/or tumor infiltrating lymphocytes.

18. The process of any one of claims 14 to 17, wherein the CD49f⁺ T-cells include CD49f⁺ memory T-cells including CD49f⁺ central memory T-cells (e.g., CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T-cells, CD49f⁺ CD27⁺ CD28⁺ CD95⁺ memory T-cells, CD49f*CD27⁺ CD28⁺ CD45RA⁺ CD95⁺ memory T-cells, CD49f⁺ CD27⁺ CD28⁺ CD95⁺ CCR7⁺ memory T-cells or CD49f⁺ CD27⁺ CD28⁺ CD45RA⁺ CD95⁺ CCR7⁺ memory T-cells), wherein the memory T-cells are optionally positive for CD127.

19. The process of any one of claims 14 to 18, wherein the CD49f⁺ T-cells have an early memory phenotype and/or a stem-like phenotype (e.g., CD49f⁺ T-cells are positive for TCF-1 (e.g., TCF-1^{hi}) and/or LEF-1 (e.g., LEF-1^{hi}) and optionally positive for one or both of Oct4 and Sox2).

20. The process of any one of claims 14 to 19, wherein the enhanced immune properties are relative to a control (e.g., a T-cell population that is not enriched for CD49f⁺ T-cells as defined above and elsewhere herein, or an isolated or CD49f⁺ T-cell enriched T-cell population as defined above and elsewhere herein).

21. The process of any one of claims 14 to 20, wherein the isolated or CD49f⁺ T-cell enriched T-cell population is autologous, allogeneic, or xenogeneic relative to a subject to whom the population is or will be administered.

22. The process of any one of claims 14 to 21, wherein the isolation or enriching steps comprises contacting the sample T-cell population with an antigen-binding molecule that binds to CD49f and isolating cells that bind to the antigen-binding molecule.

23. The process of claim 22, wherein the anti-CD49f antigen-binding molecule is directly or indirectly connected to a magnetic or paramagnetic particle.

24. The process of any one of claims 14 to 23, wherein the enriching comprises positive selection for CD49f⁺ cells using affinity based selection.

25. The process of any one of claims 14 to 24, further comprising isolating the T-cell-containing sample from a suitable source of T-cells.

26. The process of any one of claims 14 to 25, further comprising activating the T-cells of the isolated or CD49f⁺ T-cell enriched T-cell population.

27. The process of any one of claims 14 to 26, further comprising stimulating the T-cells of the isolated or CD49f⁺ T-cell enriched T-cell population to proliferate.

28. The process of claim 27, wherein the activation and stimulation of the T-cells comprises contacting the T-cells with (1) an anti-CD3 antigen-binding molecule and (2) an anti-CD28 antigen-binding molecule, or B7-1 or B7-2.

29. The process of claim 27, wherein the activation and stimulation of the T-cells comprises contacting the T-cells with an anti-CD49f antigen-binding molecule.

30. The process of any one of claims 14 to 29, further comprising contacting the T-cells with an antigen to produce antigen-specific T-cells.

31. The process of any one of claims 14 to 30, further comprising transducing the T-cells of the isolated or CD49f⁺ T-cell enriched T-cell population with a nucleic acid (e.g., a vector such as a viral vector including a retroviral vector such as a lentiviral vector) from which a rTCR or CAR is expressible, optionally in combination with a cytokine (e.g., an immune-stimulatory cytokine).

32. The process of claim 31, wherein the T-cells are transduced with the nucleic acid after T-cell proliferation.

33. The process of claim 31 or claim 32, wherein the CAR comprises a) an extracellular domain that binds to an antigen or portion thereof, wherein the antigen is selected from the group consisting of: a cancer or tumor-associated antigen, an infectious disease-associated antigen, an autoimmune disease-associated antigen, a transplantation antigen and an allergen; b) a transmembrane domain derived from a polypeptide selected from the group consisting of: CD8α, CD4, CD28, CD45, PD-1, and CD152; c) one or more intracellular costimulatory signaling domains selected from the group consisting of: CD28, CD54 (ICAM), CD134 (OX40), CD137 (4-1BB), CD152 (CTLA4), CD273 (PD-L2), CD274 (PD-L1), and CD278 (ICOS); and d) a CD3-ζ signaling domain.

34. The process of claim 33, wherein the extracellular domain comprises an antigen-binding molecule (e.g., scFv) that binds the antigen.

35. The process of claim 33 or claim 34, wherein the CAR further comprises a hinge region polypeptide (e.g., a hinge region of IgG1 or CD8α).

36. The process of any one claims 33 to 35, wherein the CAR further comprises a signal peptide (e.g., an IgG1 heavy chain signal polypeptide or a CD8a signal polypeptide).

37. The process of any one claims 14 to 36, further comprising storing the isolated or CD49f⁺ T-cell enriched T-cell population.

38. The process of claim 37, wherein the storing comprises cryopreservation of the isolated or CD49f⁺ T-cell enriched T-cell population.

39. A kit for carrying out the manufacturing processes of any one of claims 13 to 37, comprising one or more antigen-binding molecules or other binding partners, suitably coupled to solid supports, for the isolation or separation of, or enrichment for, a CD49f⁺ T-cell enriched T-cell population as defined in any one of claims 1 to 13.

40. The kit of claim 39, comprising an antigen-binding molecule for one or more T-cell biomarkers selected from CD95, CD45RA, CCR7, CD28, CD27, CD62L, CD127, and one or both of CD8 and CD4.

41. The kit of claim 39 or claim 40, further containing instructional material for carry out the isolation or separation of, or enrichment for, the CD49f+ T-cell enriched T-cell population.

42. The kit of any one of claims 39 to 40, comprising antigen-binding molecules for positive and negative selection, suitably bound to magnetic beads.

43. The kit of claim 42, comprising instructions to carry out selection starting with a sample, such as a PBMC sample, by selecting based on expression of a first surface marker, recognized by one or more of the antigen-binding molecules provided with the kit, retaining both positive and negative fractions.

44. The kit of claim 43, wherein the instructions further include instructions to carry out one or more additional selection steps, starting with the positive and/or negative fractions derived therefrom, for example, while maintaining the compositions in a contained environment and/or in the same separation vessel.

45. A method of determining a likelihood that a T-cell population is competent for immunotherapy (e.g., adoptive cell therapy), the method comprising or consisting essentially of: determining a level or concentration of CD49f⁺ T-cells in a sample of the T-cell population; and determining a likelihood that the T-cell population is competent for immunotherapy based on the level or concentration of CD49f⁺ T-cells in the sample.

46. The method of claim 45, wherein the level or concentration of CD49f⁺ T-cells comprises a level or concentration of CD49f^{hi} T-cells only, a level or concentration of CD49f^{int} T-cells only, or a level or concentration of both CD49f^{hi} T-cells and CD49f^{int} T-cells.

47. The method of claim 45 or claim 46, wherein the CD49f⁺ T-cells comprise memory T-cells (e.g., central memory T-cells), such as, but not limited to, CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T- cells, CD49f⁺ CD27⁺ CD28⁺ CD95⁺ memory T-cells, CD49f⁺ CD27⁺ CD28⁺ CD45RA⁺ CD95⁺ memory T-cells, CD49f⁺ CD27⁺ CD28⁺ CD95⁺ CCR7⁺ memory T-cells or CD49f⁺ CD27⁺ CD28⁺ CD45RA⁺ CD95⁺ CCR7⁺ memory T-cells, wherein the memory T-cells are optionally positive for CD127.

48. The method of any one of claims 45 to 47, wherein the CD49f⁺ T-cells are positive for one or both of CD4 and CD8.

49. The method of any one of claims 45 to 48, wherein the CD49f⁺ T-cells have an early memory phenotype and/or a stem-like phenotype.

50. The method of claim 49, wherein the CD49f⁺ T-cells are positive for TCF-1 (e.g., TCF-1^{hi}) and/or LEF-1 (e.g., LEF-1^{hi}) and optionally positive for one or both of Oct4 and Sox2.

51. The method of any one of claims 45 to 50, wherein the T-cell population is determined to be competent for immunotherapy when the level or concentration of CD49f⁺ T-cells meets or exceeds a threshold level or concentration that correlates with competence for immunotherapy.

52. The method of claim 51, wherein the T-cell population is determined to be competent for immunotherapy when the level or concentration of CD49f⁺ T-cells is at least 1% of the T-cells in the population (including at least 2% and up to and including 100%, and all integer percentages between 2% and 100%) of the T-cells in the population.

53. The method of claim 51, wherein the T-cell population is determined to be competent for immunotherapy when the level or concentration of CD49f⁺ T-cells is at least 1% of the T-cells in the population (including at least 2% and up to and including 100%, and all integer percentages between 2% and 100%) of the total number of cells in the T-cell population.

54. The method of any one of claims 45 to 50, wherein the T-cell population is determined to be incompetent for immunotherapy when the level or concentration of CD49f⁺ T-cells is below a threshold level or concentration that correlates with competence for immunotherapy.

55. The method of claim 54, wherein the T-cell population is determined to be incompetent for immunotherapy when the level or concentration of CD49f⁺ T-cells is less than 1% of the T-cells in the population, including less than 0.9%, less than 0.8%, less than 0.7%, less than 0.6%, less than 0.5%, less than 0.4%, less than 0.3%, less than 0.2% or less than 0.1% of the T-cells in the population.

56. The method of claim 54, wherein the T-cell population is determined to be incompetent for immunotherapy when the level or concentration of CD49f⁺ T-cells is less than 1% of the T-cells in the population, including less than 0.9%, less than 0.8%, less than 0.7%, less than 0.6%, less than 0.5%, less than 0.4%, less than 0.3%, less than 0.2% or less than 0.1% of the total number of cells in the population.

57. The method of any one of claims 45 to 56, wherein the T-cell population is an unexpanded population of T-cells.

58. The method of any one of claims 45 to 56, wherein the T-cell population is an expanded population of T-cells.

59. The method of any one of claims 45 to 58, wherein the T-cell population results from a process that includes antigen-specific stimulation of T-cells to produce antigen-specific T-cells.

60. A kit for determining a likelihood that a T-cell population is competent for immunotherapy (e.g., adoptive cell therapy), the kit comprising an antigen-binding molecule for detecting CD49f⁺ T-cells in the T-cell population.

61. The kit of claim 60, further comprising an antigen-binding molecule for one or more T-cell biomarkers selected CD95, CD45RA, CCR7, CD28, CD27, CCR7, CD45RA, CD62L, CD127 and one or both of CD8 and CD4.

62. The kit of claim 60 or claim 61, containing instructional material for detecting and/or quantifying the CD49f⁺ T-cells in the T-cell population.

63. The kit of any one of claims 60 to 62, wherein the T-cell population is a T-cell-containing sample that has not been subjected to the manufacturing process of any one of claims 14 to 38, or an isolated or CD49f⁺ T-cell enriched T-cell population as defined in any one of claims 1 to 12.

64. A pharmaceutical composition comprising an isolated or CD49f⁺ T-cell enriched T-cell population as defined in any one of claims 1 to 13 or obtained from the process of any one of claims 14 to 38, and optionally a pharmaceutically carrier.

65. An article of manufacture, comprising: one or more sealable containers individually comprising: at least one unit dose of an isolated or CD49f⁺ T-cell enriched T-cell population as defined in any one of claims 1 to 13 or as obtained from the process of any one of claims 14 to 38 for administration to a subject; packaging material; and a label or package insert comprising instructions for administering the at least one unit dose to a subject by carrying out at least one administration.

66. The article of manufacture of claim 65, wherein the unit dose comprises about 1×10⁶ to about 5×10⁸ cells.

67. The article of manufacture of claim 65 or claim 66, wherein the article of manufacture comprises a plurality of unit doses and the label or package insert comprises instructions for administering the plurality of unit doses to the subject by carrying out a first administration and at least one subsequent administration, wherein the first administration comprises delivering one of the unit doses to the subject and the at least one subsequent administration individually comprises administering one or a plurality of said the doses to the subject.

68. The article of manufacture of any one of claims 65 to 67, wherein the isolated or CD49f⁺ T-cell enriched T-cell population is autologous, allogeneic or xenogeneic relative to the subject to whom the population is administered.

69. A method for enhancing immune effector function in a patient having or at risk of developing an immune dysfunction, or requiring augmented immune effector function, the method comprising or consisting essentially of: administering to the patient an effective amount of an isolated or CD49f⁺ T-cell enriched T-cell population as defined in any one of claims 1 to 12, or as obtained by the process of any one of claims 14 to 38.

70. A method for treating or inhibiting the development of a condition in a patient, wherein the patient has or is at risk of developing an immune dysfunction and/or is in need or desirous of augmented immune effector function, the method comprising or consisting essentially of: administering to the patient an effective amount of an isolated or CD49f⁺ T-cell enriched T-cell population as defined in any one of claims 1 to 13 or as obtained from the process of any one of claims 14 to 38.

71. The method of claim 69 or claim 70, wherein the patient is in need of adoptive transfer of T-cells, suitably antigen-specific T-cells.

72. The method of any one of claims 69 to 71, wherein the isolated or CD49f⁺ T-cell enriched T-cell population is autologous to the patient.

73. The method of any one of claims 69 to 71, wherein the isolated or CD49f⁺ T-cell enriched T-cell population is from a suitable donor who is suitably HLA-matched to the patient.

74. The method of any one of claims 69 to 71, wherein the isolated or CD49f⁺ T-cell enriched T-cell population is from a xenogeneic source.

75. The method of any one of claims 69 to 74, wherein the patient has or is at risk of developing a T-cell dysfunctional disorder.

76. The method of any one of claims 69 to 75, wherein patient is a cancer patient, a patient having an infectious disease, a patient having autoimmune disease, or a patient in need of transplantation.

77. A method for enhancing immune effector function in a patient having or at risk of developing an immune dysfunction, or requiring augmented immune effector function, the method comprising or consisting essentially of: contacting T-cells in the patient with an anti-CD49f affinity agent (e.g., an anti-CD49f antigen-binding molecule) to selectively stimulate activation of CD49f⁺ immune cells in the patient and enhance immune effector function in the patient.

78. A method for treating or inhibiting the development of a condition in a patient, wherein the patient has or is at risk of developing an immune dysfunction and/or is in need or desirous of an augmented immune effector function, the method comprising or consisting essentially of: contacting T-cells in the patient with an anti-CD49f affinity agent (e.g., an anti-CD49f antigen-binding molecule) to selectively stimulate activation of CD49f⁺ immune cells in the patient and treat or inhibit the development of the condition.

79. The method of claim 78, wherein the condition is selected from cancer, infectious disease, autoimmune disease, inflammatory disease, and immunodeficiency.

80. The method of any one of claims 77 to 79, wherein the anti-CD49f affinity agent (e.g., an anti-CD49f antigen-binding molecule) stimulates activation of CD49f⁺ T-cells, which are suitably selected from CD49f⁺ memory T-cells (e.g., CD49f⁺ CD27⁺ CD28⁺ memory T-cells, CD49f⁺ CD27⁺ CD28⁺ CD45RA⁺ memory T-cells, CD49f⁺ CD27⁺ CD28⁺ CCR7⁺ memory T-cells, CD49f⁺ CD27⁺ CD28⁺ CD45RA⁺ CCR7⁺ memory T-cells, CD49f⁺ CD27⁺ CD28⁺ CD95⁺ memory T- cells, CD49f⁺ CD27⁺ CD28⁺ CD45RA⁺ CD95⁺ memory T-cells, CD49f⁺ CD27⁺ CD28⁺ CD95⁺ CCR7⁺ memory T-cells and CD49f⁺ CD27⁺ CD28⁺ CD45RA⁺ CD95⁺ CCR7⁺ memory T-cells), wherein the memory T-cells are optionally positive for CD127.

81. The method of any one of claims 77 to 80, wherein the patient has or is at risk of developing a T-cell dysfunctional disorder.

82. The method of any one of claims 77 to 81, wherein the patient is a cancer patient, a patient having an infectious disease, a patient having autoimmune disease, or a patient in need of transplantation.

83. The method of any one of claims 77 to 82, comprising administering an effective amount of the anti-CD49f affinity agent (e.g., an anti-CD49f antigen-binding molecule) to the subject.

84. The method of claim 83, method further comprising concurrently administering with the anti-CD49f affinity agent (e.g., an anti-CD49f antigen-binding molecule) an ancillary agent that stimulates immune effector function or that treats or inhibits the development of the condition in the patient.

85. The method of claim 84, wherein the ancillary agent comprises an immunotherapy such as an immune-checkpoint inhibitor.
